SEQUENCE LISTING (139 sequences)

SEQ ID NO: 1 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 2 - protein, 9 aa, Homo sapiens
IMIGVLVGV

SEQ ID NO: 3 - protein, 5 aa, Artificial Sequence
  TCR alpha chain variable domain CDR-1
TSITA

SEQ ID NO: 4 - protein, 6 aa, Artificial Sequence
  TCR alpha chain variable domain CDR-2
IRSNER

SEQ ID NO: 5 - protein, 11 aa, Artificial Sequence
  TCR alpha chain variable domain CDR-3
ATDLTSGGNYK

SEQ ID NO: 6 - protein, 11 aa, Artificial Sequence
  TCR alpha chain variable domain CDR-3
ATDFTSGGNYK

SEQ ID NO: 7 - protein, 11 aa, Artificial Sequence
  TCR alpha chain variable domain CDR-3
ATDLTTGGNYK

SEQ ID NO: 8 - protein, 11 aa, Artificial Sequence
  TCR alpha chain variable domain CDR-3
ATDFTTGGNYK

SEQ ID NO: 9 - protein, 5 aa, Artificial Sequence
  TCR beta chain variable domain CDR-1
KGHPV

SEQ ID NO: 10 - protein, 6 aa, Artificial Sequence
  TCR beta chain variable domain CDR-2
FQNQEV

SEQ ID NO: 11 - protein, 11 aa, Artificial Sequence
  TCR beta chain variable domain CDR-3
ASSLGLGDYEQ

SEQ ID NO: 12 - protein, 11 aa, Artificial Sequence
  TCR beta chain variable domain CDR-3
ASSLGTGDYEQ

SEQ ID NO: 13 - DNA, 75 bp, Homo sapiens
gactttattctggaagatgctgcttctccaaagtgcattatgaaggaaaaaaaaaagcctggtgagactttcttc

SEQ ID NO: 14 - protein, 25 aa, Homo sapiens
DFILEDAASPKCIMKEKKKPGETFF

SEQ ID NO: 15 - protein, 701 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 16 - protein, 598 aa, Artificial Sequence
  CT 548 pLenti 1 CEA TCR TRAV8-1*01 118P & 119T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDLTSGGNYKPTFGKGTSLVVHPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGLGDYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 17 - protein, 598 aa, Artificial Sequence
  CT 549 pLenti 1 CEA TCR TRAV8-1*01 118P & 119T TRBV26*01 L117T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDLTSGGNYKPTFGKGTSLVVHPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGTGDYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 18 - protein, 598 aa, Artificial Sequence
  CT 550 pLenti 1 CEA TCR TRAV8-1*01 118P & 119T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDLTSGGNYKPTFGKGTSLVVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGLGDYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 19 - protein, 598 aa, Artificial Sequence
  CT 551 pLenti 1 CEA TCR TRAV8-1*01 L110F 118P & 119T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDFTSGGNYKPTFGKGTSLVVHPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGLGDYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 20 - protein, 598 aa, Artificial Sequence
  CT 552 pLenti 1 CEA TCR TRAV8-1*01 S112T 118P & 119T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDLTTGGNYKPTFGKGTSLVVHPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGLGDYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 21 - protein, 598 aa, Artificial Sequence
  CT 553 pLenti 1 CEA TCR TRAV8-1*01 L110F S112T 118P & 119T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDFTTGGNYKPTFGKGTSLVVHPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGLGDYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 22 - protein, 598 aa, Artificial Sequence
  CT 554 pLenti 1 CEA TCR TRAV8-1*01 L110F 118P & 119T TRBV26*01 L117T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDFTSGGNYKPTFGKGTSLVVHPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGTGDYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 23 - protein, 598 aa, Artificial Sequence
  CT 555 pLenti 1 CEA TCR TRAV8-1*01 S112T 118P & 119T TRBV26*01 L117T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDLTTGGNYKPTFGKGTSLVVHPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGTGDYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 24 - protein, 598 aa, Artificial Sequence
  CT 556 pLenti 1 CEA TCR TRAV8-1*01 L110F S112T 118P & 119T TRBV26*01 L117T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDFTTGGNYKPTFGKGTSLVVHPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGTGDYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 25 - protein, 598 aa, Artificial Sequence
  CT 557 pLenti 1 CEA TCR TRAV8-1*01 L110F 118P & 119T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDFTSGGNYKPTFGKGTSLVVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGLGDYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 26 - protein, 598 aa, Artificial Sequence
  CT 558 pLenti 1 CEA TCR TRAV8-1*01 S112T 118P & 119T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDLTTGGNYKPTFGKGTSLVVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGLGDYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 27 - protein, 598 aa, Artificial Sequence
  CT 559 pLenti 1 CEA TCR TRAV8-1*01 L110F S112T 118P & 119T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDFTTGGNYKPTFGKGTSLVVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGLGDYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 28 - protein, 598 aa, Artificial Sequence
  CT 560 pLenti 1 CEA TCR TRAV8-1*01 118P & 119T TRBV26*01 L117T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDLTSGGNYKPTFGKGTSLVVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGTGDYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 29 - protein, 598 aa, Artificial Sequence
  CT 561 pLenti 1 CEA TCR TRAV8-1*01 L110F 118P & 119T TRBV26*01 L117T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDFTSGGNYKPTFGKGTSLVVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGTGDYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 30 - protein, 598 aa, Artificial Sequence
  CT 562 pLenti 1 CEA TCR TRAV8-1*01 S112T 118P & 119T TRBV26*01 L117T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDLTTGGNYKPTFGKGTSLVVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGTGDYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 31 - protein, 598 aa, Artificial Sequence
  CT 563 pLenti 1 CEA TCR TRAV8-1*01 L110F S112T 118P & 119T TRBV26*01 L117T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDFTTGGNYKPTFGKGTSLVVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGTGDYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 32 - protein, 19 aa, Unknown
  P2A self-cleaving peptide
ATNFSLLKQAGDVEENPGP

SEQ ID NO: 33 - protein, 18 aa, Unknown
  T2A self-cleaving peptide
EGRGSLLTCGDVEENPGP

SEQ ID NO: 34 - protein, 20 aa, Unknown
  E2A self-cleaving peptide
QCTNYALLKLAGDVESNPGP

SEQ ID NO: 35 - protein, 22 aa, Unknown
  F2A self-cleaving peptide
VKQTLNFDLLKLAGDVESNPGP

SEQ ID NO: 36 - protein, 596 aa, Artificial Sequence
  CT 532 pLenti 1 CEA TCR TRAV8-1*01 TRBV26*01 with regular murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDLTSGGNYKFGKGTSLVVHPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGLGDYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 37 - protein, 596 aa, Artificial Sequence
  CT 533 pLenti 1 CEA TCR TRAV8-1*01 TRBV26*01 L117T with regular murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDLTSGGNYKFGKGTSLVVHPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGTGDYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 38 - protein, 596 aa, Artificial Sequence
  CT 534 pLenti 1 CEA TCR TRAV8-1*01 TRBV26*01 murine constant region (no PT)
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDLTSGGNYKFGKGTSLVVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGLGDYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 39 - protein, 596 aa, Artificial Sequence
  CT 535 pLenti 1 CEA TCR TRAV8-1*01 L110F TRBV26*01 with regular murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDFTSGGNYKFGKGTSLVVHPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGLGDYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 40 - protein, 596 aa, Artificial Sequence
  CT 536 pLenti 1 CEA TCR TRAV8-1*01 S112T TRBV26*01 with regular murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDLTTGGNYKFGKGTSLVVHPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGLGDYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 41 - protein, 596 aa, Artificial Sequence
  CT 537 pLenti 1 CEA TCR TRAV8-1*01 L110F & S112T TRBV26*01 with regular murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDFTTGGNYKFGKGTSLVVHPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGLGDYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 42 - protein, 596 aa, Artificial Sequence
  CT 538 pLenti 1 CEA TCR TRAV8-1*01 L110F TRBV26*01 L117T with regular murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDFTSGGNYKFGKGTSLVVHPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGTGDYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 43 - protein, 596 aa, Artificial Sequence
  CT 539 pLenti 1 CEA TCR TRAV8-1*01 S112T TRBV26*01 L117T with regular murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDLTTGGNYKFGKGTSLVVHPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGTGDYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 44 - protein, 596 aa, Artificial Sequence
  CT 540 pLenti 1 CEA TCR TRAV8-1*01 L110F & S112T TRBV26*01 L117T with regular murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDFTTGGNYKFGKGTSLVVHPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGTGDYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 45 - protein, 596 aa, Artificial Sequence
  CT 541 pLenti 1 CEA TCR TRAV8-1*01 L110F TRBV26*01 with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDFTSGGNYKFGKGTSLVVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGLGDYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 46 - protein, 596 aa, Artificial Sequence
  CT 542 pLenti 1 CEA TCR TRAV8-1*01 S112T TRBV26*01 with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDLTTGGNYKFGKGTSLVVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGLGDYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 47 - protein, 596 aa, Artificial Sequence
  CT 543 pLenti 1 CEA TCR TRAV8-1*01 L110F & S112T TRBV26*01 with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDFTTGGNYKFGKGTSLVVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGLGDYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 48 - protein, 596 aa, Artificial Sequence
  CT 544 pLenti 1 CEA TCR  TRAV8-1*01 TRBV26*01 L117T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDLTSGGNYKFGKGTSLVVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGTGDYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 49 - protein, 596 aa, Artificial Sequence
  CT 545 pLenti 1 CEA TCR TRAV8-1*01 L110F TRBV26*01 L117T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDFTSGGNYKFGKGTSLVVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGTGDYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 50 - protein, 596 aa, Artificial Sequence
  CT 546 pLenti 1 CEA TCR TRAV8-1*01 S112T TRBV26*01 L117T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDLTTGGNYKFGKGTSLVVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGTGDYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 51 - protein, 596 aa, Artificial Sequence
  CT 547 pLenti 1 CEA TCR TRAV8-1*01 L110F & S112T TRBV26*01 L117T with murine constant region
MHSLLGLLMVSLWLQLTRVNSQLAEENPWALSVHEGESVTVNCSYKTSITALQWYRQKSGEGPAQLILIRSNEREKRNGRLRATLDTSSQSSSLSITATRCEDTAVYFCATDFTTGGNYKFGKGTSLVVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSGSGATNFSLLKQAGDVEENPGPMATRLLCYTVLCLLGARILNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLGTGDYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 52 - protein, 535 aa, Artificial Sequence
  CAR with ScFv; CT 618
MDMRVPAQLLGLLLLWLRGARCQVQLVQSGSELKKPGASVKVSCKASGYTFTEFGMNWVRQAPGQGLEWMGWINTKTGEATYVEEFKGRFVFSLDTSVSTAYLQISSLKAEDTAVYYCARWDFAYYVEAMDYWGQGTTVTVSSGGGGSGGGGSGGGGSGGDIQMTQSPSSLSASVGDRVTITCKASQNVGTNVAWYQQKPGKAPKLLIYSASYRYSGVPSRFSGSGSGTDFTLTISSLQPEDFATYYCHQYYTYPLFTFGQGTKLEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 537 aa, Artificial Sequence
  CAR with ScFv; CT 619
MDMRVPAQLLGLLLLWLRGARCQVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSGGGGSGGGGSGGGGSGGDIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 536 aa, Artificial Sequence
  CAR with ScFv; CT 620
MDMRVPAQLLGLLLLWLRGARCQVQLVQSGSELKKPGASVKVSCKASGYTFTEFGMNWVRQAPGQGLEWMGWINTKTGEATYVEEFKGRFVFSLDTSVSTAYLQISSLKAEDTAVYYCARWDFAHYFQTMDYWGQGTTVTVSSGGGGSGGGGSGGGGSGGDIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 5 aa, Artificial Sequence
  ScFv CDR-H1
EFGMN

SEQ ID NO: 56 - protein, 17 aa, Artificial Sequence
  ScFv CDR-H2
WINTKTGEATYVEEFKG

SEQ ID NO: 57 - protein, 12 aa, Artificial Sequence
  ScFv CDR-H3
WDFAYYVEAMDY

SEQ ID NO: 58 - protein, 12 aa, Artificial Sequence
  ScFv CDR-H3
WDFAHYFQTMDY

SEQ ID NO: 59 - protein, 11 aa, Artificial Sequence
  ScFv CDR-L1
KASQNVGTNVA

SEQ ID NO: 60 - protein, 11 aa, Artificial Sequence
  ScFv CDR-L1
KASAAVGTYVA

SEQ ID NO: 61 - protein, 7 aa, Artificial Sequence
  ScFv CDR-L2
SASYRYS

SEQ ID NO: 62 - protein, 7 aa, Artificial Sequence
  ScFv CDR-L2
SASYRKR

SEQ ID NO: 63 - protein, 10 aa, Artificial Sequence
  ScFv CDR-L3
HQYYTYPLFT

SEQ ID NO: 64 - protein, 243 aa, Artificial Sequence
  ScFv that target CEA; MFE23M
QVQLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSSGGGGSGGGGSGGGGSGGENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELK

SEQ ID NO: 65 - protein, 243 aa, Artificial Sequence
  ScFv that target CEA; MFE23H
QVQLVQSGAEVKKPGASVKVSCKASGFNIKDSYMHWVRQAPGQGLEWMGWIDPENGDTEYAPKFQGRVTMTTDTSTSTAYMELRSLRSDDTAVYYCNEGTPTGPYYFDYWGQGTTVTVSSGGGGSGGGGSGGGGSGGEIVLTQSPATLSLSPGERATLSCSASSSVSYMHWYQQKPGLAPRLLIYSTSNLASGIPDRFSGSGSGTDFTLTISRLEPEDFAVYYCQQRSSYPLTFGQGTKLEIK

SEQ ID NO: 66 - protein, 243 aa, Artificial Sequence
  ScFv that target CEA; E8
EVQLAESGGGLVQPGGSLRLSCAASGFTFSSDAMSWVRQAPGKGLEWVSAISGSGGSTYYADSVKGRFTISRDNSKNTLYLQMNSLRAEDTAVYYCAKSNEFLFDYWGQGTLVTVSSGGGGSGGGGSGGGGSGGSSELTQDPAVSVALGQTVRITCQGDSLRSSYASWYRQRPGQAPVLVIYGKNNRPSGIPDRFSGSSSGNTASLTITGAQAEDEADYYWNSSYAWLPYVVFGGGTKLTVLG

SEQ ID NO: 67 - protein, 243 aa, Artificial Sequence
  ScFv that target CEA; SM3E
QVQLEQSGAGVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSSGGGGSGGGGSGGGGSGGENVLTQSPSSMSVSVGDRVNIACSASSSVPYMHWLQQKPGKSPKLLIYLTSNLASGVPSRFSGSGSGTDYSLTISSVQPEDAATYYCQQRSSYPLTFGGGTKLEIK

SEQ ID NO: 68 - protein, 246 aa, Artificial Sequence
  ScFv that target CEA; CT618
QVQLVQSGSELKKPGASVKVSCKASGYTFTEFGMNWVRQAPGQGLEWMGWINTKTGEATYVEEFKGRFVFSLDTSVSTAYLQISSLKAEDTAVYYCARWDFAYYVEAMDYWGQGTTVTVSSGGGGSGGGGSGGGGSGGDIQMTQSPSSLSASVGDRVTITCKASQNVGTNVAWYQQKPGKAPKLLIYSASYRYSGVPSRFSGSGSGTDFTLTISSLQPEDFATYYCHQYYTYPLFTFGQGTKLEIK

SEQ ID NO: 69 - protein, 246 aa, Artificial Sequence
  ScFv that target CEA; CT619
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSGGGGSGGGGSGGGGSGGDIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 70 - protein, 246 aa, Artificial Sequence
  ScFv that target CEA; CT620
QVQLVQSGSELKKPGASVKVSCKASGYTFTEFGMNWVRQAPGQGLEWMGWINTKTGEATYVEEFKGRFVFSLDTSVSTAYLQISSLKAEDTAVYYCARWDFAHYFQTMDYWGQGTTVTVSSGGGGSGGGGSGGGGSGGDIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 71 - protein, 45 aa, Unknown
  CD8alpha hinge
TTTPAPRPPTPAPTIASQPLSLRPEACRPAAGGAVHTRGLDFACD

SEQ ID NO: 72 - DNA, 135 bp, Unknown
  CD8alpha hinge
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 73 - protein, 41 aa, Unknown
  CD28 hinge
CTIEVMYPPPYLDNEKSNGTIIHVKGKHLCPSPLFPGPSKP

SEQ ID NO: 74 - DNA, 123 bp, Unknown
  CD28 hinge
tgtaccattgaagttatgtatcctcctccttacctagacaatgagaagagcaatggaaccattatccatgtgaaagggaaacacctttgtccaagtcccctatttcccggaccttctaagccc

SEQ ID NO: 75 - protein, 27 aa, Unknown
  CD28 transmembrane domain
FWVLVVVGGVLACYSLLVTVAFIIFWV

SEQ ID NO: 76 - DNA, 81 bp, Unknown
  CD28 transmembrane domain
ttctgggtgctggtcgttgtgggcggcgtgctggcctgctacagcctgctggtgacagtggccttcatcatcttttgggtg

SEQ ID NO: 77 - protein, 25 aa, Unknown
  IL-2Rbeta transmembrane domain
IPWLGHLLVGLSGAFGFIILVYLLI

SEQ ID NO: 78 - DNA, 75 bp, Unknown
  IL-2Rbeta transmembrane domain
attccgtggctcggccacctcctcgtgggcctcagcggggcttttggcttcatcatcttagtgtacttgctgatc

SEQ ID NO: 79 - protein, 112 aa, Unknown
  CD3zeta activation domain
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - DNA, 336 bp, Unknown
  CD3zeta activation domain
agagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 81 - protein, 43 aa, Unknown
  CD3zeta activation domain ITAM
RVKFSRSADAPAYQQGQNQLYNELNLGRREEYDVLHMQALPPR

SEQ ID NO: 82 - DNA, 129 bp, Unknown
  CD3zeta activation domain ITAM
agagtgaagttcagcaggagcgcagacgcccccgcgtaccagcagggccagaaccagctctataacgagctcaatctaggacgaagagaggagtacgatgttttgcacatgcaggccctgccccctcgc

SEQ ID NO: 83 - protein, 41 aa, Unknown
  CD28 co-stimulatory domain
RSKRSRLLHSDYMNMTPRRPGPTRKHYQPYAPPRDFAAYRS

SEQ ID NO: 84 - DNA, 123 bp, Unknown
  CD28 co-stimulatory domain
aggagcaagcggagcagactgctgcacagcgactacatgaacatgaccccccggaggcctggccccacccggaagcactaccagccctacgcccctcccagggatttcgccgcctaccggagc

SEQ ID NO: 85 - protein, 23 aa, Unknown
  TCR alpha transmembrane domain
VIGFRILLLKVAGFNLLMTLRLW

SEQ ID NO: 86 - DNA, 69 bp, Unknown
  TCR alpha transmembrane domain
gtgattgggttccgaatcctcctcctgaaagtggccgggtttaatctgctcatgacgctgcggctgtgg

SEQ ID NO: 87 - protein, 23 aa, Unknown
  TCR beta transmembrane domain
TILYEILLGKATLYAVLVSALVL

SEQ ID NO: 88 - DNA, 69 bp, Unknown
  TCR beta transmembrane domain
accatcctctatgagatcttgctagggaaggccaccttgtatgccgtgctggtcagtgccctcgtgctg

SEQ ID NO: 89 - protein, 10 aa, Unknown
  TCR beta intracellular domain
MAMVKRKDSR

SEQ ID NO: 90 - DNA, 30 bp, Unknown
  TCR beta intracellular domain
atggccatggtcaagagaaaggattccaga

SEQ ID NO: 91 - protein, 509 aa, Homo sapiens
MTVTYTARVANARFGGFSQLLLLWRGSIYKLLWRELLCFLGFYMALSAAYRFVLTEGQKRYFEKLVIYCDQYASLIPVSFVLGFYVTLVVNRWWSQYLCMPLPDALMCVVAGTVHGRDDRGRLYRRTLMRYAGLSAVLILRSVSTAVFKRFPTIDHVVEAGFMTREERKKFENLNSSYNKYWVPCVWFSNLAAQARREGRIRDNSALKLLLEELNVFRGKCGMLFHYDWISVPLVYTQVVTIALYSYFLACLIGRQFLDPAQGYKDHDLDLCVPIFTLLQFFFYAGWLKVAEQLINPFGEDDDDFETNFLIDRNFQVSMLAVDEMYDDLAVLEKDLYWDAAEARAPYTAATVFQLRQPSFQGSTFDITLAKEDMQFQRLDGLDGPMGEAPGDFLQRLLPAGAGMVAGGPLGRRLSFLLRKNSCVSEASTGASCSCAVVPEGAAPECSCGDPLLDPGLPEPEAPPPAGPEPLTLIPGPVEPFSIVTMPGPRGPAPPWLPSPIGEEEENLA

SEQ ID NO: 92 - protein, 473 aa, Homo sapiens
MTVSYTLKVAEARFGGFSGLLLRWRGSIYKLLYKEFLLFGALYAVLSITYRLLLTQEQRYVYAQVARYCNRSADLIPLSFVLGFYVTLVVNRWWSQYTSIPLPDQLMCVISASVHGVDQRGRLLRRTLIRYANLASVLVLRSVSTRVLKRFPTMEHVVDAGFMSQEERKKFESLKSDFNKYWVPCVWFTNLAAQARRDGRIRDDIALCLLLEELNKYRAKCSMLFHYDWISIPLVYTQVVTIAVYSFFALSLVGRQFVEPEAGAAKPQKLLKPGQEPAPALGDPDMYVPLTTLLQFFFYAGWLKVAEQIINPFGEDDDDFETNQLIDRNLQVSLLSVDEMYQNLPPAEKDQYWDEDQPQPPYTVATAAESLRPSFLGSTFNLRMSDDPEQSLQVEASPGSGRPAPAAQTPLLGRFLGVGAPSPAISLRNFGRVRGTPRPPHLLRFRAEEGGDPEAAARIEEESAESGDEALEP

SEQ ID NO: 93 - protein, 495 aa, Homo sapiens
MENKAMYLHTVSDCDTSSICEDSFDGRSLSKLNLCEDGPCHKRRASICCTQLGSLSALKHAVLGLYLLVFLILVGIFILAVSRPRSSPDDLKALTRNVNRLNESFRDLQLRLLQAPLQADLTEQVWKVQDALQNQSDSLLALAGAVQRLEGALWGLQAQAVQTEQAVALLRDRTGQQSDTAQLELYQLQVESNSSQLLLRRHAGLLDGLARRVGILGEELADVGGVLRGLNHSLSYDVALHRTRLQDLRVLVSNASEDTRRLRLAHVGMELQLKQELAMLNAVTEDLRLKDWEHSIALRNISLAKGPPGPKGDQGDEGKEGRPGIPGLPGLRGLPGERGTPGLPGPKGDDGKLGATGPMGMRGFKGDRGPKGEKGEKGDRAGDASGVEAPMMIRLVNGSGPHEGRVEVYHDRRWGTVCDDGWDKKDGDVVCRMLGFRGVEEVYRTARFGQGTGRIWMDDVACKGTEETIFRCSFSKWGVTNCGHAEDASVTCNRH

SEQ ID NO: 94 - protein, 998 aa, Homo sapiens
MVFQTRYPSWIILCYIWLLRFAHTGEAQAAKEVLLLDSKAQQTELEWISSPPNGWEEISGLDENYTPIRTYQVCQVMEPNQNNWLRTNWISKGNAQRIFVELKFTLRDCNSLPGVLGTCKETFNLYYYETDYDTGRNIRENLYVKIDTIAADESFTQGDLGERKMKLNTEVREIGPLSKKGFYLAFQDVGACIALVSVKVYYKKCWSIIENLAIFPDTVTGSEFSSLVEVRGTCVSSAEEEAENAPRMHCSAEGEWLVPIGKCICKAGYQQKGDTCEPCGRGFYKSSSQDLQCSRCPTHSFSDKEGSSRCECEDGYYRAPSDPPYVACTRPPSAPQNLIFNINQTTVSLEWSPPADNGGRNDVTYRILCKRCSWEQGECVPCGSNIGYMPQQTGLEDNYVTVMDLLAHANYTFEVEAVNGVSDLSRSQRLFAAVSITTGQAAPSQVSGVMKERVLQRSVELSWQEPEHPNGVITEYEIKYYEKDQRERTYSTVKTKSTSASINNLKPGTVYVFQIRAFTAAGYGNYSPRLDVATLEEATGKMFEATAVSSEQNPVIIIAVVAVAGTIILVFMVFGFIIGRRHCGYSKADQEGDEELYFHFKFPGTKTYIDPETYEDPNRAVHQFAKELDASCIKIERVIGAGEFGEVCSGRLKLPGKRDVAVAIKTLKVGYTEKQRRDFLCEASIMGQFDHPNVVHLEGVVTRGKPVMIVIEFMENGALDAFLRKHDGQFTVIQLVGMLRGIAAGMRYLADMGYVHRDLAARNILVNSNLVCKVSDFGLSRVIEDDPEAVYTTTGGKIPVRWTAPEAIQYRKFTSASDVWSYGIVMWEVMSYGERPYWDMSNQDVIKAIEEGYRLPAPMDCPAGLHQLMLDCWQKERAERPKFEQIVGILDKMIRNPNSLKTPLGTCSRPISPLLDQNTPDFTTFCSVGEWLQAIKMERYKDNFTAAGYNSLESVARMTIEDVMSLGITLVGHQKKIMSSIQTMRAQMLHLHGTGIQV

SEQ ID NO: 95 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 96 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 97 - DNA, 1800 bp, Artificial Sequence
  CT 548 pLenti 1 CEA TCR TRAV8-1*01 118P & 119T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatctaacctcaggaggaaactacaaacctacgtttgggaaagggaccagcctcgtggttcatccagacattcagaaccccgaaccagccgtatatcagttgaaggacccaagatctcaggatagtacactctgtttgtttacggactttgactcacaaatcaacgtcccgaagactatggaaagtggtacgttcatcacagataagacggttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgtcagtgatgggtctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgggactgggggactatgaacagtacttcggtcccggcaccaggctcacggttttagaggatctccggaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtccactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 98 - DNA, 1800 bp, Artificial Sequence
  CT 549 pLenti 1 CEA TCR TRAV8-1*01 118P & 119T TRBV26*01 L117T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatctaacctcaggaggaaactacaaacctacgtttgggaaagggaccagcctcgtggttcatccagacattcagaaccccgaaccagccgtatatcagttgaaggacccaagatctcaggatagtacactctgtttgtttacggactttgactcacaaatcaacgtcccgaagactatggaaagtggtacgttcatcacagataagacggttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgtcagtgatgggtctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctggggacaggggactatgaacagtacttcggtcccggcaccaggctcacggttttagaggatctccggaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtccactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 99 - DNA, 1800 bp, Artificial Sequence
  CT 550 pLenti 1 CEA TCR TRAV8-1*01 118P & 119T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatctaacctcaggaggaaactacaaacctacgtttgggaaagggaccagcctcgtggttcatccaaatattcagaaccccgaaccagccgtatatcagttgaaggacccaagatctcaggatagtacactctgtttgtttacggactttgactcacaaatcaacgtcccgaagactatggaaagtggtacgttcatcacagataagtgcgttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgctggtgatcgttctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgggactgggggactatgaacagtacttcggtcccggcaccaggctcacggttttagaggacctgcgcaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtgcactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 100 - DNA, 1800 bp, Artificial Sequence
  CT 551 pLenti 1 CEA TCR TRAV8-1*01 L110F 118P & 119T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatttcacctcaggaggaaactacaaacctacgtttgggaaagggaccagcctcgtggttcatccagacattcagaaccccgaaccagccgtatatcagttgaaggacccaagatctcaggatagtacactctgtttgtttacggactttgactcacaaatcaacgtcccgaagactatggaaagtggtacgttcatcacagataagacggttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgtcagtgatgggtctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgggactgggggactatgaacagtacttcggtcccggcaccaggctcacggttttagaggatctccggaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtccactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 101 - DNA, 1800 bp, Artificial Sequence
  CT 552 pLenti 1 CEA TCR TRAV8-1*01 S112T 118P & 119T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatctaaccaccggaggaaactacaaacctacgtttgggaaagggaccagcctcgtggttcatccagacattcagaaccccgaaccagccgtatatcagttgaaggacccaagatctcaggatagtacactctgtttgtttacggactttgactcacaaatcaacgtcccgaagactatggaaagtggtacgttcatcacagataagacggttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgtcagtgatgggtctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgggactgggggactatgaacagtacttcggtcccggcaccaggctcacggttttagaggatctccggaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtccactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 102 - DNA, 1800 bp, Artificial Sequence
  CT 553 pLenti 1 CEA TCR TRAV8-1*01 L110F S112T 118P & 119T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatttcaccaccggaggaaactacaaacctacgtttgggaaagggaccagcctcgtggttcatccagacattcagaaccccgaaccagccgtatatcagttgaaggacccaagatctcaggatagtacactctgtttgtttacggactttgactcacaaatcaacgtcccgaagactatggaaagtggtacgttcatcacagataagacggttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgtcagtgatgggtctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgggactgggggactatgaacagtacttcggtcccggcaccaggctcacggttttagaggatctccggaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtccactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 103 - DNA, 1800 bp, Artificial Sequence
  CT 554 pLenti 1 CEA TCR TRAV8-1*01 L110F 118P & 119T TRBV26*01 L117T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatttcacctcaggaggaaactacaaacctacgtttgggaaagggaccagcctcgtggttcatccagacattcagaaccccgaaccagccgtatatcagttgaaggacccaagatctcaggatagtacactctgtttgtttacggactttgactcacaaatcaacgtcccgaagactatggaaagtggtacgttcatcacagataagacggttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgtcagtgatgggtctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctggggacaggggactatgaacagtacttcggtcccggcaccaggctcacggttttagaggatctccggaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtccactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 104 - DNA, 1800 bp, Artificial Sequence
  CT 555 pLenti 1 CEA TCR TRAV8-1*01 S112T 118P & 119T TRBV26*01 L117T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatctaaccaccggaggaaactacaaacctacgtttgggaaagggaccagcctcgtggttcatccagacattcagaaccccgaaccagccgtatatcagttgaaggacccaagatctcaggatagtacactctgtttgtttacggactttgactcacaaatcaacgtcccgaagactatggaaagtggtacgttcatcacagataagacggttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgtcagtgatgggtctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctggggacaggggactatgaacagtacttcggtcccggcaccaggctcacggttttagaggatctccggaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtccactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 105 - DNA, 1800 bp, Artificial Sequence
  CT 556 pLenti 1 CEA TCR TRAV8-1*01 L110F S112T 118P & 119T TRBV26*01 L117T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatttcaccaccggaggaaactacaaacctacgtttgggaaagggaccagcctcgtggttcatccagacattcagaaccccgaaccagccgtatatcagttgaaggacccaagatctcaggatagtacactctgtttgtttacggactttgactcacaaatcaacgtcccgaagactatggaaagtggtacgttcatcacagataagacggttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgtcagtgatgggtctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctggggacaggggactatgaacagtacttcggtcccggcaccaggctcacggttttagaggatctccggaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtccactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 106 - DNA, 1800 bp, Artificial Sequence
  CT 557 pLenti 1 CEA TCR TRAV8-1*01 L110F 118P & 119T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatttcacctcaggaggaaactacaaacctacgtttgggaaagggaccagcctcgtggttcatccaaatattcagaaccccgaaccagccgtatatcagttgaaggacccaagatctcaggatagtacactctgtttgtttacggactttgactcacaaatcaacgtcccgaagactatggaaagtggtacgttcatcacagataagtgcgttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgctggtgatcgttctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgggactgggggactatgaacagtacttcggtcccggcaccaggctcacggttttagaggacctgcgcaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtgcactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 107 - DNA, 1800 bp, Artificial Sequence
  CT 558 pLenti 1 CEA TCR TRAV8-1*01 S112T 118P & 119T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatctaaccaccggaggaaactacaaacctacgtttgggaaagggaccagcctcgtggttcatccaaatattcagaaccccgaaccagccgtatatcagttgaaggacccaagatctcaggatagtacactctgtttgtttacggactttgactcacaaatcaacgtcccgaagactatggaaagtggtacgttcatcacagataagtgcgttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgctggtgatcgttctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgggactgggggactatgaacagtacttcggtcccggcaccaggctcacggttttagaggacctgcgcaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtgcactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 108 - DNA, 1800 bp, Artificial Sequence
  CT 559 pLenti 1 CEA TCR TRAV8-1*01 L110F S112T 118P & 119T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatttcaccaccggaggaaactacaaacctacgtttgggaaagggaccagcctcgtggttcatccaaatattcagaaccccgaaccagccgtatatcagttgaaggacccaagatctcaggatagtacactctgtttgtttacggactttgactcacaaatcaacgtcccgaagactatggaaagtggtacgttcatcacagataagtgcgttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgctggtgatcgttctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgggactgggggactatgaacagtacttcggtcccggcaccaggctcacggttttagaggacctgcgcaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtgcactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 109 - DNA, 1800 bp, Artificial Sequence
  CT 560 pLenti 1 CEA TCR TRAV8-1*01 118P & 119T TRBV26*01 L117T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatctaacctcaggaggaaactacaaacctacgtttgggaaagggaccagcctcgtggttcatccaaatattcagaaccccgaaccagccgtatatcagttgaaggacccaagatctcaggatagtacactctgtttgtttacggactttgactcacaaatcaacgtcccgaagactatggaaagtggtacgttcatcacagataagtgcgttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgctggtgatcgttctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctggggacaggggactatgaacagtacttcggtcccggcaccaggctcacggttttagaggacctgcgcaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtgcactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 110 - DNA, 1800 bp, Artificial Sequence
  CT 561 pLenti 1 CEA TCR TRAV8-1*01 L110F 118P & 119T TRBV26*01 L117T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatttcacctcaggaggaaactacaaacctacgtttgggaaagggaccagcctcgtggttcatccaaatattcagaaccccgaaccagccgtatatcagttgaaggacccaagatctcaggatagtacactctgtttgtttacggactttgactcacaaatcaacgtcccgaagactatggaaagtggtacgttcatcacagataagtgcgttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgctggtgatcgttctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctggggacaggggactatgaacagtacttcggtcccggcaccaggctcacggttttagaggacctgcgcaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtgcactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 111 - DNA, 1800 bp, Artificial Sequence
  CT 562 pLenti 1 CEA TCR TRAV8-1*01 S112T 118P & 119T TRBV26*01 L117T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatctaaccaccggaggaaactacaaacctacgtttgggaaagggaccagcctcgtggttcatccaaatattcagaaccccgaaccagccgtatatcagttgaaggacccaagatctcaggatagtacactctgtttgtttacggactttgactcacaaatcaacgtcccgaagactatggaaagtggtacgttcatcacagataagtgcgttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgctggtgatcgttctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctggggacaggggactatgaacagtacttcggtcccggcaccaggctcacggttttagaggacctgcgcaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtgcactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 112 - DNA, 1800 bp, Artificial Sequence
  CT 563 pLenti 1 CEA TCR TRAV8-1*01 L110F S112T 118P & 119T TRBV26*01 L117T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatttcaccaccggaggaaactacaaacctacgtttgggaaagggaccagcctcgtggttcatccaaatattcagaaccccgaaccagccgtatatcagttgaaggacccaagatctcaggatagtacactctgtttgtttacggactttgactcacaaatcaacgtcccgaagactatggaaagtggtacgttcatcacagataagtgcgttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgctggtgatcgttctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctggggacaggggactatgaacagtacttcggtcccggcaccaggctcacggttttagaggacctgcgcaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtgcactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 113 - DNA, 1794 bp, Artificial Sequence
  CT 532 pLenti 1 CEA TCR TRAV8-1*01 TRBV26*01 with regular murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatctaacctcaggaggaaactacaaatttgggaaagggaccagcctcgtggttcatccagacattcagaaccccgaaccagccgtatatcagttgaaggacccaagatctcaggatagtacactctgtttgtttacggactttgactcacaaatcaacgtcccgaagactatggaaagtggtacgttcatcacagataagacggttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgtcagtgatgggtctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgggactgggggactatgaacagtacttcggtcccggcaccaggctcacggttttagaggatctccggaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtccactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 114 - DNA, 1794 bp, Artificial Sequence
  CT 533 pLenti 1 CEA TCR TRAV8-1*01 TRBV26*01 L117T with regular murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatctaacctcaggaggaaactacaaatttgggaaagggaccagcctcgtggttcatccagacattcagaaccccgaaccagccgtatatcagttgaaggacccaagatctcaggatagtacactctgtttgtttacggactttgactcacaaatcaacgtcccgaagactatggaaagtggtacgttcatcacagataagacggttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgtcagtgatgggtctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctggggacaggggactatgaacagtacttcggtcccggcaccaggctcacggttttagaggatctccggaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtccactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 115 - DNA, 1794 bp, Artificial Sequence
  CT 534 pLenti 1 CEA TCR TRAV8-1*01 TRBV26*01 murine constant region (no PT)
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatctaacctcaggaggaaactacaaatttgggaaagggaccagcctcgtggttcatccaaatattcagaaccccgaaccagccgtatatcagttgaaggacccaagatctcaggatagtacactctgtttgtttacggactttgactcacaaatcaacgtcccgaagactatggaaagtggtacgttcatcacagataagtgcgttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgctggtgatcgttctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgggactgggggactatgaacagtacttcggtcccggcaccaggctcacggttttagaggacctgcgcaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtgcactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 116 - DNA, 1794 bp, Artificial Sequence
  CT 535 pLenti 1 CEA TCR TRAV8-1*01 L110F TRBV26*01 with regular murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatttcacctcaggaggaaactacaaatttgggaaagggaccagcctcgtggttcatccagacattcagaaccccgaaccagccgtatatcagttgaaggacccaagatctcaggatagtacactctgtttgtttacggactttgactcacaaatcaacgtcccgaagactatggaaagtggtacgttcatcacagataagacggttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgtcagtgatgggtctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgggactgggggactatgaacagtacttcggtcccggcaccaggctcacggttttagaggatctccggaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtccactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 117 - DNA, 1794 bp, Artificial Sequence
  CT 536 pLenti 1 CEA TCR TRAV8-1*01 S112T TRBV26*01 with regular murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatctaaccaccggaggaaactacaaatttgggaaagggaccagcctcgtggttcatccagacattcagaaccccgaaccagccgtatatcagttgaaggacccaagatctcaggatagtacactctgtttgtttacggactttgactcacaaatcaacgtcccgaagactatggaaagtggtacgttcatcacagataagacggttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgtcagtgatgggtctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgggactgggggactatgaacagtacttcggtcccggcaccaggctcacggttttagaggatctccggaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtccactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 118 - DNA, 1794 bp, Artificial Sequence
  CT 537 pLenti 1 CEA TCR TRAV8-1*01 L110F & S112T TRBV26*01 with regular murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatttcaccaccggaggaaactacaaatttgggaaagggaccagcctcgtggttcatccagacattcagaaccccgaaccagccgtatatcagttgaaggacccaagatctcaggatagtacactctgtttgtttacggactttgactcacaaatcaacgtcccgaagactatggaaagtggtacgttcatcacagataagacggttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgtcagtgatgggtctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgggactgggggactatgaacagtacttcggtcccggcaccaggctcacggttttagaggatctccggaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtccactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 119 - DNA, 1794 bp, Artificial Sequence
  CT 538 pLenti 1 CEA TCR TRAV8-1*01 L110F TRBV26*01 L117T with regular murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatttcacctcaggaggaaactacaaatttgggaaagggaccagcctcgtggttcatccagacattcagaaccccgaaccagccgtatatcagttgaaggacccaagatctcaggatagtacactctgtttgtttacggactttgactcacaaatcaacgtcccgaagactatggaaagtggtacgttcatcacagataagacggttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgtcagtgatgggtctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctggggacaggggactatgaacagtacttcggtcccggcaccaggctcacggttttagaggatctccggaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtccactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 120 - DNA, 1794 bp, Artificial Sequence
  CT 539 pLenti 1 CEA TCR TRAV8-1*01 S112T TRBV26*01 L117T with regular murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatctaaccaccggaggaaactacaaatttgggaaagggaccagcctcgtggttcatccagacattcagaaccccgaaccagccgtatatcagttgaaggacccaagatctcaggatagtacactctgtttgtttacggactttgactcacaaatcaacgtcccgaagactatggaaagtggtacgttcatcacagataagacggttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgtcagtgatgggtctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctggggacaggggactatgaacagtacttcggtcccggcaccaggctcacggttttagaggatctccggaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtccactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 121 - DNA, 1794 bp, Artificial Sequence
  CT 540 pLenti 1 CEA TCR TRAV8-1*01 L110F & S112T TRBV26*01 L117T with regular murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatttcaccaccggaggaaactacaaatttgggaaagggaccagcctcgtggttcatccagacattcagaaccccgaaccagccgtatatcagttgaaggacccaagatctcaggatagtacactctgtttgtttacggactttgactcacaaatcaacgtcccgaagactatggaaagtggtacgttcatcacagataagacggttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgtcagtgatgggtctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctggggacaggggactatgaacagtacttcggtcccggcaccaggctcacggttttagaggatctccggaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtccactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 122 - DNA, 1794 bp, Artificial Sequence
  CT 541 pLenti 1 CEA TCR TRAV8-1*01 L110F TRBV26*01 with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatttcacctcaggaggaaactacaaatttgggaaagggaccagcctcgtggttcatccaaatattcagaaccccgaaccagccgtatatcagttgaaggacccaagatctcaggatagtacactctgtttgtttacggactttgactcacaaatcaacgtcccgaagactatggaaagtggtacgttcatcacagataagtgcgttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgctggtgatcgttctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgggactgggggactatgaacagtacttcggtcccggcaccaggctcacggttttagaggacctgcgcaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtgcactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 123 - DNA, 1794 bp, Artificial Sequence
  CT 542 pLenti 1 CEA TCR TRAV8-1*01 S112T TRBV26*01 with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatctaaccaccggaggaaactacaaatttgggaaagggaccagcctcgtggttcatccaaatattcagaaccccgaaccagccgtatatcagttgaaggacccaagatctcaggatagtacactctgtttgtttacggactttgactcacaaatcaacgtcccgaagactatggaaagtggtacgttcatcacagataagtgcgttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgctggtgatcgttctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgggactgggggactatgaacagtacttcggtcccggcaccaggctcacggttttagaggacctgcgcaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtgcactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 124 - DNA, 1794 bp, Artificial Sequence
  CT 543 pLenti 1 CEA TCR TRAV8-1*01 L110F & S112T TRBV26*01 with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatttcaccaccggaggaaactacaaatttgggaaagggaccagcctcgtggttcatccaaatattcagaaccccgaaccagccgtatatcagttgaaggacccaagatctcaggatagtacactctgtttgtttacggactttgactcacaaatcaacgtcccgaagactatggaaagtggtacgttcatcacagataagtgcgttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgctggtgatcgttctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgggactgggggactatgaacagtacttcggtcccggcaccaggctcacggttttagaggacctgcgcaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtgcactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 125 - DNA, 1794 bp, Artificial Sequence
  CT 544 pLenti 1 CEA TCR  TRAV8-1*01 TRBV26*01 L117T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatctaacctcaggaggaaactacaaatttgggaaagggaccagcctcgtggttcatccaaatattcagaaccccgaaccagccgtatatcagttgaaggacccaagatctcaggatagtacactctgtttgtttacggactttgactcacaaatcaacgtcccgaagactatggaaagtggtacgttcatcacagataagtgcgttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgctggtgatcgttctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctggggacaggggactatgaacagtacttcggtcccggcaccaggctcacggttttagaggacctgcgcaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtgcactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 126 - DNA, 1794 bp, Artificial Sequence
  CT 545 pLenti 1 CEA TCR TRAV8-1*01 L110F TRBV26*01 L117T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatttcacctcaggaggaaactacaaatttgggaaagggaccagcctcgtggttcatccaaatattcagaaccccgaaccagccgtatatcagttgaaggacccaagatctcaggatagtacactctgtttgtttacggactttgactcacaaatcaacgtcccgaagactatggaaagtggtacgttcatcacagataagtgcgttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgctggtgatcgttctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctggggacaggggactatgaacagtacttcggtcccggcaccaggctcacggttttagaggacctgcgcaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtgcactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 127 - DNA, 1794 bp, Artificial Sequence
  CT 546 pLenti 1 CEA TCR TRAV8-1*01 S112T TRBV26*01 L117T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatctaaccaccggaggaaactacaaatttgggaaagggaccagcctcgtggttcatccaaatattcagaaccccgaaccagccgtatatcagttgaaggacccaagatctcaggatagtacactctgtttgtttacggactttgactcacaaatcaacgtcccgaagactatggaaagtggtacgttcatcacagataagtgcgttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgctggtgatcgttctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctggggacaggggactatgaacagtacttcggtcccggcaccaggctcacggttttagaggacctgcgcaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtgcactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 128 - DNA, 1794 bp, Artificial Sequence
  CT 547 pLenti 1 CEA TCR TRAV8-1*01 L110F & S112T TRBV26*01 L117T with murine constant region
atgcacagcctcctggggttgttgatggtgtcactgtggctgcaactgacaagggtgaatagtcaactagcagaagagaatccgtgggccctgagcgtccacgagggtgaaagtgtcacggtgaattgtagttacaagacatccataactgccctacagtggtacagacagaagtcaggcgaaggccctgcccagctaatcttaatacgttcaaatgagagagagaagcgcaatggaagactcagagccacccttgacacctccagccagagcagctccttgtccatcactgctactcggtgtgaagacaccgctgtgtacttctgtgctactgatttcaccaccggaggaaactacaaatttgggaaagggaccagcctcgtggttcatccaaatattcagaaccccgaaccagccgtatatcagttgaaggacccaagatctcaggatagtacactctgtttgtttacggactttgactcacaaatcaacgtcccgaagactatggaaagtggtacgttcatcacagataagtgcgttctggacatgaaggctatggactcaaagagcaacggggcaattgcttggtccaaccagacaagctttacctgtcaggacatttttaaggagactaatgctacttatccctccagcgacgttccgtgtgatgcgactcttaccgagaagtcttttgagaccgatatgaatctcaacttccagaatctgctggtgatcgttctgcggatcctgcttctgaaggttgcaggattcaatcttcttatgactctccggctctggtcttcaggttctggcgcgacgaattttagtttgcttaagcaagccggagatgtggaggaaaatcctggaccgatggctacaaggctcctctgttacacagtactttgtctcctgggtgcaagaattttgaactcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctggggacaggggactatgaacagtacttcggtcccggcaccaggctcacggttttagaggacctgcgcaacgtcaccccaccaaaggtcagtttgtttgagccatcaaaggcggagatcgccaacaaacagaaagctacgctcgtgtgtttggctcggggcttcttcccagaccacgtagaactttcctggtgggtcaatggaaaggaggttcattccggagtgtgcactgatccccaagcgtacaaggaatccaactatagctactgtctctcatctcggctccgggtgagtgcgacattctggcataatcctcggaaccactttcgatgccaagtgcagtttcatgggttgagcgaggaagacaagtggcccgagggcagtcctaaaccagtcactcaaaacataagcgccgaggcatggggtagagccgattgtgggattactagcgcttcataccaacaaggggtattgagcgctacaattctttacgaaattctcctcggcaaggcgacgctctacgccgtactggtgtctactctcgtggttatggcaatggtgaaacggaaaaacagctaatag

SEQ ID NO: 129 - DNA, 1608 bp, Artificial Sequence
  CAR with ScFv; CT 618
atggacatgagggtccccgctcagctcctggggctcctgctactctggctccgaggtgccagatgtcaggtgcagctggtgcaatctgggtctgagttgaagaagcctggggcctcagtgaaggtttcctgcaaggcttctggatacaccttcactgagtttggaatgaactgggtgcgacaggcccctggacaagggcttgagtggatgggatggataaacaccaaaactggagaggcaacatatgttgaagagtttaagggacggtttgtcttctccttggacacctctgtcagcacggcatatctgcagatcagcagcctaaaggctgaagacactgccgtgtattactgtgcgagatgggacttcgcttattacgtggaggctatggactactggggccaagggaccacggtcaccgtctcctcaggcggaggtggaagcggagggggaggatctggcggcggaggaagcggaggcgatatccagatgacccagtctccatcctccctgtctgcatctgtgggagacagagtcaccatcacttgcaaggccagtcagaatgtgggtactaatgttgcctggtatcagcagaaaccagggaaagcacctaagctcctgatctattcggcatcctaccgctacagtggagtcccatcaaggttcagtggcagtggatctgggacagatttcactctcaccatcagcagtctgcaacctgaagatttcgcaacttactactgtcaccaatattacacctatcctctattcacgtttggccagggcaccaagctcgagatcaagacaacgacgccagctccccgcccgccaacccctgcacctacgattgcatcacaaccgctgtccctgcggcctgaagcttgtcgcccagccgcaggtggcgccgtacatacacgggggctggattttgcctgtgatttctgggtgctggtcgttgtgggcggcgtgctggcctgctacagcctgctggtgacagtggccttcatcatcttttgggtgaggagcaagcggagtcgactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgctag

SEQ ID NO: 130 - DNA, 1614 bp, Artificial Sequence
  CAR with ScFv; CT 619
atggacatgagggtccccgctcagctcctggggctcctgctactctggctccgaggtgccagatgtcaggtgcagctggtgcagtctggcgccgaagtgaagaaacctggagctagtgtgaaggtgtcctgcaaggccagcggctacaccttcaccgagttcggcatgaactgggtccgacaggctccaggccagggcctcgaatggatgggctggatcaacaccaagaccggcgaggccacctacgtggaagagttcaagggcagagtgaccttcaccacggacaccagcaccagcaccgcctacatggaactgcggagcctgagaagcgacgacaccgccgtgtactactgcgccagatgggacttcgcttattacgtggaagccatggactactggggccagggcaccaccgtgaccgtgtctagcggcggaggtggaagcggagggggaggatctggcggcggaggaagcggaggcgatatccagatgacccagtctccatcctccctgtctgcatctgtgggagacagagtcaccatcacttgcaaggccagtgcggctgtgggtacgtatgttgcgtggtatcagcagaaaccagggaaagcacctaagctcctgatctattcggcatcctaccgcaaaaggggagtcccatcaaggttcagtggcagtggatctgggacagatttcactctcaccatcagcagtctgcaacctgaagatttcgcaacttactactgtcaccaatattacacctatcctctattcacgtttggccagggcaccaagctcgagatcaagcgtacgacaacgacgccagctccccgcccgccaacccctgcacctacgattgcatcacaaccgctgtccctgcggcctgaagcttgtcgcccagccgcaggtggcgccgtacatacacgggggctggattttgcctgtgatttctgggtgctggtcgttgtgggcggcgtgctggcctgctacagcctgctggtgacagtggccttcatcatcttttgggtgaggagcaagcggagtcgactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgctag

SEQ ID NO: 131 - DNA, 1611 bp, Artificial Sequence
  CAR with ScFv; CT 620
atggacatgagggtccccgctcagctcctggggctcctgctactctggctccgaggtgccagatgtcaggtgcagctggtgcaatctgggtctgagttgaagaagcctggggcctcagtgaaggtttcctgcaaggcttctggatacaccttcactgagtttggaatgaactgggtgcgacaggcccctggacaagggcttgagtggatgggatggataaacaccaaaactggagaggcaacatatgttgaagagtttaagggacggtttgtcttctccttggacacctctgtcagcacggcatatctgcagatcagcagcctaaaggctgaagacactgccgtgtattactgtgcgagatgggactttgctcattactttcagactatggactactggggccaagggaccacggtcaccgtctcctcaggcggaggtggaagcggagggggaggatctggcggcggaggaagcggaggcgatatccagatgacccagtctccatcctccctgtctgcatctgtgggagacagagtcaccatcacttgcaaggccagtgcggctgtgggtacgtatgttgcgtggtatcagcagaaaccagggaaagcacctaagctcctgatctattcggcatcctaccgcaaaaggggagtcccatcaaggttcagtggcagtggatctgggacagatttcactctcaccatcagcagtctgcaacctgaagatttcgcaacttactactgtcaccaatattacacctatcctctattcacgtttggccagggcaccaagctcgagatcaagcgtacaacgacgccagctccccgcccgccaacccctgcacctacgattgcatcacaaccgctgtccctgcggcctgaagcttgtcgcccagccgcaggtggcgccgtacatacacgggggctggattttgcctgtgatttctgggtgctggtcgttgtgggcggcgtgctggcctgctacagcctgctggtgacagtggccttcatcatcttttgggtgaggagcaagcggagtcgactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgctag

SEQ ID NO: 132 - protein, 729 aa, Artificial Sequence
  ScFv that target CEA; MFE23M
CAGGTCCAGCTGCAGCAGTCTGGGGCAGAGCTTGTGAGGTCAGGGACCTCAGTCAAGTTGTCCTGCACAGCTTCTGGCTTCAACATTAAAGACTCCTATATGCACTGGTTGAGGCAGGGGCCTGAACAGGGCCTGGAGTGGATTGGATGGATTGATCCTGAGAATGGTGATACTGAATATGCCCCGAAGTTCCAGGGCAAGGCCACTTTTACTACAGACACATCCTCCAACACAGCCTACCTGCAGCTCAGCAGCCTGACATCTGAGGACACTGCCGTCTATTACTGTAATGAAGGGACACCGACAGGGCCATACTATTTTGACTACTGGGGTCAAGGAACCACAGTCACCGTGTCCTCAGGCGGAGGTGGAAGCGGAGGGGGAGGATCTGGCGGCGGAGGAAGCGGAGGCGAGAACGTTCTCACCCAGTCTCCAGCAATCATGTCTGCATCTCCAGGGGAGAAGGTCACCATAACCTGCAGTGCCAGCTCAAGTGTAAGTTACATGCACTGGTTCCAGCAGAAGCCAGGCACTTCTCCCAAACTCTGGATTTATAGCACATCCAACCTGGCTTCTGGAGTCCCTGCTCGCTTCAGTGGCAGTGGATCTGGGACCTCTTACTCTCTCACAATCAGCCGAATGGAGGCTGAAGATGCTGCCACTTATTACTGCCAGCAAAGGAGTAGTTACCCGCTCACGTTCGGTGCTGGGACCAAGCTGGAGCTGAAA

SEQ ID NO: 133 - protein, 729 aa, Artificial Sequence
  ScFv that target CEA; MFE23H
CAGGTCCAGCTGGTGCAGTCTGGGGCAGAGGTGAAGAAACCAGGGGCCTCAGTCAAGGTGTCCTGCAAAGCTTCTGGCTTCAACATTAAAGACTCCTATATGCACTGGGTGAGGCAGGCGCCTGGACAGGGCCTGGAGTGGATGGGATGGATTGATCCTGAGAATGGTGATACTGAATATGCCCCGAAGTTCCAGGGCAGGGTCACTATGACTACAGACACATCCACCTCCACAGCCTACATGGAGCTCAGGAGCCTGAGATCTGACGACACTGCCGTCTATTACTGTAATGAAGGGACACCGACAGGGCCATACTATTTTGACTACTGGGGTCAAGGAACCACAGTCACCGTGTCCTCAGGCGGAGGTGGAAGCGGAGGGGGAGGATCTGGCGGCGGAGGAAGCGGAGGCGAGATCGTTCTCACCCAGTCTCCAGCAACCTTGTCTCTGTCTCCAGGGGAGAGGGCCACCCTAAGCTGCAGTGCCAGCTCAAGTGTAAGTTACATGCACTGGTACCAGCAGAAGCCAGGCCTTGCTCCCAGACTCCTGATTTATAGCACATCCAACCTGGCTTCTGGAATCCCTGATCGCTTCAGTGGCAGTGGATCTGGGACCGATTTCACTCTCACAATCAGCCGACTGGAGCCTGAAGATTTCGCCGTTTATTACTGCCAGCAAAGGAGTAGTTACCCGCTCACGTTCGGTCAGGGGACCAAGCTGGAGATCAAA

SEQ ID NO: 134 - protein, 729 aa, Artificial Sequence
  ScFv that target CEA; E8
GAGGTGCAGCTGGCGGAGTCTGGGGGAGGCTTGGTACAGCCTGGGGGGTCCCTGAGACTCTCCTGTGCAGCCTCTGGATTCACCTTTAGCAGCGATGCCATGAGCTGGGTCCGCCAGGCTCCAGGGAAGGGGCTGGAGTGGGTCTCAGCTATTAGTGGTAGTGGTGGTAGCACATACTACGCAGACTCCGTGAAGGGCCGGTTCACCATCTCCAGAGACAATTCCAAGAACACGCTGTATCTGCAAATGAACAGCCTGAGAGCCGAGGACACGGCCGTGTATTACTGTGCAAAGTCTAATGAGTTTCTTTTTGACTACTGGGGCCAAGGTACCCTGGTCACCGTGTCGAGTGGCGGAGGTGGAAGCGGAGGGGGAGGATCTGGCGGCGGAGGAAGCGGAGGCTCTTCTGAGCTGACTCAGGACCCTGCTGTGTCTGTGGCCTTGGGACAGACAGTCAGGATCACATGCCAAGGAGACAGCCTCAGAAGCTCTTATGCAAGCTGGTACCGGCAGAGGCCAGGACAGGCCCCTGTACTTGTCATCTATGGTAAAAACAACCGGCCCTCAGGGATCCCAGACCGATTCTCTGGCTCCAGCTCAGGAAACACAGCTTCCTTGACCATCACTGGGGCTCAGGCGGAAGATGAGGCTGACTATTACTGGAACTCCAGCTACGCTTGGCTGCCCTACGTGGTATTCGGCGGAGGGACCAAGCTGACCGTCCTAGGT

SEQ ID NO: 135 - protein, 729 aa, Artificial Sequence
  ScFv that target CEA; SM3E
CAGGTCCAGCTGGAGCAGTCTGGGGCAGGGGTTGTGAAGCCAGGGGCCTCAGTCAAGTTGTCCTGCAAAGCTTCTGGCTTCAACATTAAAGACTCCTATATGCACTGGTTGAGGCAGGGGCCTGGACAGCGCCTGGAGTGGATTGGATGGATTGATCCTGAGAATGGTGATACTGAATATGCCCCGAAGTTCCAGGGCAAGGCCACTTTTACTACAGACACATCCGCCAACACAGCCTACCTGGGGCTCAGCAGCCTGAGACCTGAGGACACTGCCGTCTATTACTGTAATGAAGGGACACCGACAGGGCCATACTATTTTGACTACTGGGGTCAAGGAACCCTAGTCACCGTGTCCTCAGGCGGAGGTGGAAGCGGAGGGGGAGGATCTGGCGGCGGAGGAAGCGGAGGCGAGAACGTTCTCACCCAGTCTCCAAGCTCTATGTCTGTATCTGTCGGGGACAGGGTCAACATCGCCTGCAGTGCCAGCTCAAGTGTACCTTACATGCACTGGCTCCAGCAGAAGCCAGGCAAATCTCCCAAACTCCTGATTTATCTCACATCCAACCTGGCTTCTGGAGTCCCTAGCCGCTTCAGTGGCAGTGGATCTGGGACCGATTACTCTCTCACAATCAGCTCAGTGCAGCCTGAAGATGCTGCCACTTATTACTGCCAGCAAAGGAGTAGTTACCCGCTCACGTTCGGTGGTGGGACCAAGCTGGAGATCAAA

SEQ ID NO: 136 - protein, 738 aa, Artificial Sequence
  ScFv that target CEA; CT618
CAGGTGCAGCTGGTGCAATCTGGGTCTGAGTTGAAGAAGCCTGGGGCCTCAGTGAAGGTTTCCTGCAAGGCTTCTGGATACACCTTCACTGAGTTTGGAATGAACTGGGTGCGACAGGCCCCTGGACAAGGGCTTGAGTGGATGGGATGGATAAACACCAAAACTGGAGAGGCAACATATGTTGAAGAGTTTAAGGGACGGTTTGTCTTCTCCTTGGACACCTCTGTCAGCACGGCATATCTGCAGATCAGCAGCCTAAAGGCTGAAGACACTGCCGTGTATTACTGTGCGAGATGGGACTTCGCTTATTACGTGGAGGCTATGGACTACTGGGGCCAAGGGACCACGGTCACCGTCTCCTCAGGCGGAGGTGGAAGCGGAGGGGGAGGATCTGGCGGCGGAGGAAGCGGAGGCGATATCCAGATGACCCAGTCTCCATCCTCCCTGTCTGCATCTGTGGGAGACAGAGTCACCATCACTTGCAAGGCCAGTCAGAATGTGGGTACTAATGTTGCCTGGTATCAGCAGAAACCAGGGAAAGCACCTAAGCTCCTGATCTATTCGGCATCCTACCGCTACAGTGGAGTCCCATCAAGGTTCAGTGGCAGTGGATCTGGGACAGATTTCACTCTCACCATCAGCAGTCTGCAACCTGAAGATTTCGCAACTTACTACTGTCACCAATATTACACCTATCCTCTATTCACGTTTGGCCAGGGCACCAAGCTCGAGATCAAG

SEQ ID NO: 137 - protein, 738 aa, Artificial Sequence
  ScFv that target CEA; CT619
CAGGTGCAGCTGGTGCAGTCTGGCGCCGAAGTGAAGAAACCTGGAGCTAGTGTGAAGGTGTCCTGCAAGGCCAGCGGCTACACCTTCACCGAGTTCGGCATGAACTGGGTCCGACAGGCTCCAGGCCAGGGCCTCGAATGGATGGGCTGGATCAACACCAAGACCGGCGAGGCCACCTACGTGGAAGAGTTCAAGGGCAGAGTGACCTTCACCACGGACACCAGCACCAGCACCGCCTACATGGAACTGCGGAGCCTGAGAAGCGACGACACCGCCGTGTACTACTGCGCCAGATGGGACTTCGCTTATTACGTGGAAGCCATGGACTACTGGGGCCAGGGCACCACCGTGACCGTGTCTAGCGGCGGAGGTGGAAGCGGAGGGGGAGGATCTGGCGGCGGAGGAAGCGGAGGCGATATCCAGATGACCCAGTCTCCATCCTCCCTGTCTGCATCTGTGGGAGACAGAGTCACCATCACTTGCAAGGCCAGTGCGGCTGTGGGTACGTATGTTGCGTGGTATCAGCAGAAACCAGGGAAAGCACCTAAGCTCCTGATCTATTCGGCATCCTACCGCAAAAGGGGAGTCCCATCAAGGTTCAGTGGCAGTGGATCTGGGACAGATTTCACTCTCACCATCAGCAGTCTGCAACCTGAAGATTTCGCAACTTACTACTGTCACCAATATTACACCTATCCTCTATTCACGTTTGGCCAGGGCACCAAGCTCGAGATCAAG

SEQ ID NO: 138 - protein, 738 aa, Artificial Sequence
  ScFv that target CEA; CT620
CAGGTGCAGCTGGTGCAATCTGGGTCTGAGTTGAAGAAGCCTGGGGCCTCAGTGAAGGTTTCCTGCAAGGCTTCTGGATACACCTTCACTGAGTTTGGAATGAACTGGGTGCGACAGGCCCCTGGACAAGGGCTTGAGTGGATGGGATGGATAAACACCAAAACTGGAGAGGCAACATATGTTGAAGAGTTTAAGGGACGGTTTGTCTTCTCCTTGGACACCTCTGTCAGCACGGCATATCTGCAGATCAGCAGCCTAAAGGCTGAAGACACTGCCGTGTATTACTGTGCGAGATGGGACTTTGCTCATTACTTTCAGACTATGGACTACTGGGGCCAAGGGACCACGGTCACCGTCTCCTCAGGCGGAGGTGGAAGCGGAGGGGGAGGATCTGGCGGCGGAGGAAGCGGAGGCGATATCCAGATGACCCAGTCTCCATCCTCCCTGTCTGCATCTGTGGGAGACAGAGTCACCATCACTTGCAAGGCCAGTGCGGCTGTGGGTACGTATGTTGCGTGGTATCAGCAGAAACCAGGGAAAGCACCTAAGCTCCTGATCTATTCGGCATCCTACCGCAAAAGGGGAGTCCCATCAAGGTTCAGTGGCAGTGGATCTGGGACAGATTTCACTCTCACCATCAGCAGTCTGCAACCTGAAGATTTCGCAACTTACTACTGTCACCAATATTACACCTATCCTCTATTCACGTTTGGCCAGGGCACCAAGCTCGAGATCAAG

SEQ ID NO: 139 - protein, 4 aa, Artificial Sequence
  immunoreceptor tyrosine-based activation motif
  SITE (2)..(3): Xaa is any amino acid
  SITE (4)..(4): Xaa is Leu or Ile
YXXX

(19) United States
(12) Patent Application Publication
(10) Pub. No.: US 2023/0257441 A1
(43) Pub. Date: Aug. 17, 2023

(54) COMPOSITIONS AND METHODS FOR TREATING CANCERS

(71) Applicant: A2 Biotherapeutics, Inc., Agoura Hills, CA (US)

(72) Inventors: Dora Toledo Warshaviak, Agoura Hills, CA (US); Carl Alexander Kamb, Thousand Oaks, CA (US); Xueyin Wang, Agoura Hills, CA (US); Mark L. Sandberg, Agoura Hills, CA (US); Han Xu, Agoura Hills, CA (US)

(21) Appl. No.: 18/020,837

(22) PCT Filed: Aug. 12, 2021
(86) PCT No.: PCT/US21/45698

§ 371 Date: Feb. 10, 2023

Related U.S. Application Data
(60) Provisional application No. 63/065,324, filed on Aug. 13, 2020.

(51) Int. Cl. C07K 14/725; C07K 16/28; C12N 5/0783; A61P 35/00

(52) U.S. Cl. CPC C07K 14/7051; C07K 16/2833; C12N 5/0636; A61P 35/00; C07K 2317/24; C07K 2317/622; C07K 2317/565

(57) ABSTRACT

The disclosure provides immune cells comprising a first activator receptor and a second inhibitory receptor, and methods of making and using same for the treatment of cancer.

### CROSS REFERENCE TO RELATED APPLICATIONS

This application claims priority to, and benefit of, U.S. Provisional Application No. 63/065,324, filed on Aug. 13, 2020, the contents of which are incorporated by reference in their entirety herein.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: A2BI_018_01WO_SeqList_ST25.txt, date recorded: Aug. 9, 2021, file size ˜378 kilobytes).

### TECHNICAL FIELD

The disclosure relates to the fields of adoptive cell therapy and cancer therapeutics.

### BACKGROUND

Cell therapy is a powerful tool for the treatment of various diseases, particularly cancers. In conventional adoptive cell therapies, immune cells are engineered to express specific receptors, for example chimeric antigen receptors (CARs) or T cell receptors (TCRs), which direct the activity of the immune cells to cellular targets via interaction of the receptor with a ligand expressed by the target cell. Identification of suitable target molecules remains challenging, as many targets are expressed in normal tissues. This expression can lead to toxicity when the transplanted cells target normal tissues expressing target molecules. There is thus a need in the art for compositions and methods useful in the treatment of disease, particularly cancers, by adoptive cell therapy.

### SUMMARY

The disclosure provides compositions and methods for increasing the specificity of immune cells used in adoptive cell therapy. The disclosure provides immune cells comprising a two-receptor system that increases the specificity of the immune cells for target cells expressing a target antigen. The immune cells comprise a first, activator receptor that activates the immune cells in response to binding of the first receptor by the target antigen. The immune cells further comprise a second, inhibitory receptor specific to a non-target antigen. This second receptor inhibits activation of the immune cells when the second receptor is bound by the non-target antigen, even when the first receptor is bound by the target antigen.

The disclosure provides an immune cell responsive to low or no expression of a protein in a cancer cell, comprising: (a) first receptor, optionally a chimeric antigen receptor (CAR) or T cell receptor (TCR), comprising an extracellular ligand binding domain specific to a target antigen selected from: (i) a cancer cell-specific antigen, or a peptide antigen thereof in a complex with a major histocompatibility complex class I (MHC-I); or (ii) CEA cell adhesion molecule 5 (CEA), or a peptide antigen thereof in a complex with a major histocompatibility complex class I (MHC-I); and (b) a second receptor, optionally an inhibitory chimeric antigen receptor (iCAR), comprising an extracellular ligand binding domain specific to a non-target antigen selected from a is selected from bestrophin-2 (BEST2), bestrophin-4 (BEST4), scavenger receptor class A member 5 (SCARA5), ephrin type-A receptor 7 (EPHA7) and transforming growth factor beta receptor 2 (TGFBR2), or an antigen peptide thereof in a complex with a major histocompatibility complex class I (MHC-I), wherein the non-target antigen is expressed at a lower level by a population of target cells than by a population of non-target cells.

In some embodiments of the immune cells of the disclosure, the target antigen is a cancer cell-specific antigen. In some embodiments, the target antigen is a peptide antigen of a cancer cell-specific antigen in a complex with a major histocompatibility complex class I (MHC-I).

In some embodiments of the immune cells of the disclosure, the cancer cell is a colorectal cancer cell. In some embodiments, the cancer cell is a pancreatic cancer cell, esophageal cancer cell, gastric cancer cell, lung adenocarcinoma cell, head-and-neck cancer cell, diffuse large B cell cancer cell, or acute myeloid leukemia cancer cell.

In some embodiments of the immune cells of the disclosure, the target antigen is CEA. In some embodiments, the target antigen is a peptide antigen of CEA in a complex with a major histocompatibility complex class I (MHC-I).

In some embodiments of the immune cells of the disclosure, the target antigen is expressed by a target cell. In some embodiments, the non-target antigen is not expressed by the target cell. In some embodiments, the non-target antigen is expressed by a non-target cell. In some embodiments, the non-target cell is a healthy cell. In some embodiments, the non-target antigen is expressed at a lower level by the target cell than the non-target cell. In some embodiments, the non-target antigen expression level is at least about 10 times less, about 30 times less, about 50 times less, about 70 times less, about 90 times less, about 100 times less, or about 110 times less in the target cell than in the non-target cell. In some embodiments, the non-target antigen expression level is at least about 5 times less in the target cell than in the non-target cell. In some embodiments, the non-target cell is a colon cell.

In some embodiments of the immune cells of the disclosure, the first receptor and the second receptor together specifically activate the immune cell in the presence of the target cell.

In some embodiments of the immune cells of the disclosure, the immune cell is a T cell. In some embodiments, the immune cell is a CD8+CD4−T cell.

In some embodiments of the immune cells of the disclosure, the CEA comprises a sequence that shares at least 95% identity to SEQ ID NO: 1. In some embodiments, the peptide antigen of CEA is IMIGVLVGV (SEQ ID NO: 2). In some embodiments, the MHC-I comprises a human leukocyte antigen A*02 allele (HLA-A*02).

In some embodiments of the immune cells of the disclosure, the first receptor is a T cell receptor (TCR). In some embodiments, the first receptor is a chimeric antigen receptor (CAR). In some embodiments, the extracellular ligand binding domain of the first receptor comprises an antibody fragment, a single chain Fv antibody fragment (ScFv), or a β chain variable domain (Vβ).

In some embodiments of the immune cells of the disclosure, the extracellular ligand binding domain of the first receptor comprises a TCR α chain variable domain and a TCR β chain variable domain. In some embodiments, the extracellular ligand binding domain of the first receptor comprises complement determining regions (CDRs) selected from SEQ ID NOs: 3-12. In some embodiments, (a) the TCR α chain variable domain comprises a CDR-1 of TSITA (SEQ ID NO: 3), a CDR-2 of IRSNER (SEQ ID NO: 4) and a CDR-3 comprising ATDLTSGGNYK (SEQ ID NO: 5), ATDFTSGGNYK (SEQ ID NO: 6), ATDLTTGGNYK (SEQ ID NO: 7) or ATDFTTGGNYK (SEQ ID NO: 8); and (b) the TCR β chain variable domain comprises a CDR-1 of KGHPV (SEQ ID NO: 9), a CDR-2 of FQNQEV (SEQ ID NO: 10), and a CDR-3 of ASSLGLGDYEQ (SEQ ID NO: 11) or ASSLGTGDYEQ (SEQ ID NO: 12). In some embodiments, (a) the TCR α chain variable domain comprises a CDR-1 of SEQ ID NO: 9, a CDR-2 of SEQ ID NO: 10 and a CDR-3 of SEQ ID NO: 11 or SEQ ID NO: 12; and (b) the TCR β chain variable domain comprises a CDR-1 of SEQ ID NO: 3, a CDR-2 of SEQ ID NO: 4 and a CDR-3 comprising SEQ ID NO: 5, SEQ ID NO: 6, SEQ ID NO: 7 or SEQ ID NO: 8.

In some embodiments of the immune cells of the disclosure, the extracellular ligand binding domain of the first receptor comprises an ScFv. the ScFv comprises CDRs selected from SEQ ID NOs: 55-63. In some embodiments, the ScFv comprises a CDR-H1 of EFGMN (SEQ ID NO: 55), a CDR-H2 of WINTKTGEATYVEEFKG (SEQ ID NO: 56), a CDR-H3 of WDFAYYVEAMDY (SEQ ID NO: 57) or WDFAHYFQTMDY (SEQ ID NO: 58), a CDR-L1 of KASQNVGTNVA (SEQ ID NO: 59) or KASAAVGTYVA (SEQ ID NO: 60), a CDR-L2 of SASYRYS (SEQ ID NO: 61) or SASYRKR (SEQ ID NO: 62), and a CDR-L3 of HQYYTYPLFT (SEQ ID NO: 63). In some embodiments, the ScFv comprises a sequence selected from SEQ ID NOs: 64-70 or a sequence having at least 85%, at least 90%, at least 95%, at least 97% or at least 99% identity thereto. In some embodiments, the ScFv comprises a sequence selected from SEQ ID NOs: 64-70.

The disclosure provides pharmaceutical compositions comprising a therapeutically effective amount of the immune cells of the disclosure. In some embodiments, the pharmaceutical compositions further comprise a pharmaceutically acceptable carrier, diluent or excipient.

The disclosure provides pharmaceutical compositions comprising the immune cells of the disclosure, for use as a medicament in the treatment of cancer.

The disclosure provides a polynucleotide system, comprising one or more polynucleotides comprising polynucleotide sequences encoding: (a) a first receptor, optionally a chimeric antigen receptor (CAR) or T cell receptor (TCR), comprising an extracellular ligand binding domain specific to a target antigen selected from: (i) a cancer cell-specific antigen, or a peptide antigen thereof in a complex with a major histocompatibility complex class I (MHC-I); or (ii) CEA cell adhesion molecule 5 (CEA), or a peptide antigen thereof in a complex with a major histocompatibility complex class I (MHC-I); and (b) a second receptor, optionally an inhibitory chimeric antigen receptor (iCAR), comprising an extracellular ligand binding domain specific to a non-target antigen selected from selected from BEST2, BEST4, SCARA5, EPHA7, and TGFBR2, or an antigen peptide thereof in a complex with a major histocompatibility complex class I (MHC-I), wherein the non-target antigen is expressed at a lower level by a population of target cells than by a population of non-target cells.

The disclosure provides vectors comprising the one or more polynucleotides of the polynucleotide system described herein.

The disclosure provides methods of killing a plurality of cancer cells and/or treating cancer in a subject, comprising administering to the subject an effective amount of the immune cells or the pharmaceutical compositions of the disclosure. In some embodiments, a plurality of cancer cells express the target antigen. In some embodiments, the plurality of cancer cells do not express the non-target antigen. In some embodiments, the plurality of cancer cells express the non-target antigen at a lower level than a plurality of healthy cells. In some embodiments, a plurality of healthy cells express both the target antigen and the non-target antigen. In some embodiments, the plurality of cancer cells express the non-target antigen at a level that is at least about 10 times less, about 30 times less, about 50 times less, about 70 times less, about 90 times less, about 100 times less, or about 110 times less than the plurality of healthy cells. In some embodiments, the non-target antigen expression level is at least about 5 times less in the plurality of cancer cells than in the plurality of healthy cells. In some embodiments, the plurality of healthy cells comprise colon cells. In some embodiments, the methods further comprise measuring the expression level of the non-target antigen in a plurality of cancer cells, and treating the subject when the expression level of the non-target antigen in the plurality of cancer cells is less than the expression level of the non-target antigen in the plurality of cancer cells is less than the expression level of the non-target antigen a plurality of healthy cells.

The disclosure provides methods of making a plurality of immune cells, comprising: (a) providing a plurality of immune cells, and (b) transforming the plurality of immune cells with the polynucleotide system or vectors of the disclosure.

The disclosure provides kits comprising the immune cells or pharmaceutical compositions of the disclosure. In some embodiments, the kits further comprise instructions for use.

### BRIEF DESCRIPTION OF THE DRAWINGS

FIG. 1 is a plot that shows the expression of CEA (CEACAM5) in normal tissues.

FIG. 2 is a plot that shows the expression of CEA across all TCGA cancers (with tumor and normal samples). Abbreviations: BLCA (Bladder cancer), BRCA (Breast Cancer), CESC (Cervical squamous cell carcinoma and endocervical adenocarcinoma), CHOL Cholangiocarcinoma), COAD (Colon adenocarcinoma), ESCA (Esophageal carcinoma), GBN (Glioblastoma multiforme), HNSC (Head and Neck squamous cell carcinoma), KICH (Kidney Chromophobe), KIRP (Kidney renal papillary cell carcinoma), LIHC (Liver hepatocellular carcinoma), LUAD (Lung adenocarcinoma), LUSC (Lung squamous cell carcinoma), PAAD (Pancreatic adenocarcinoma), PRAD (Prostate adenocarcinoma), PCPG (Pheochromocytoma and Paraganglioma), READ (Rectum adenocarcinoma), SARC (Sarcoma), SKCM (Skin Cutaneous Melanoma), THCA (Thyroid carcinoma), STAD (Stomach adenocarcinoma), UCEC (Uterine Corpus Endometrial Carcinoma).

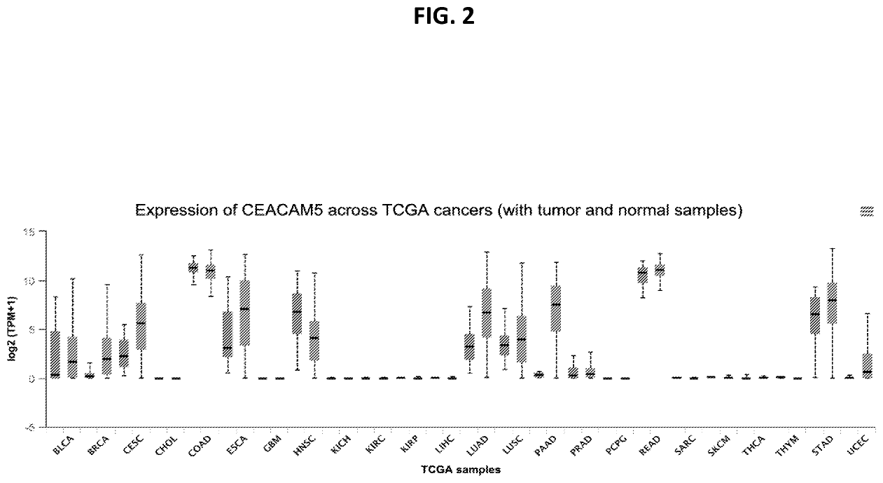

FIG. 3 is a table showing estimated deaths in the U.S. by cancer site, statistics taken from the American Cancer Society.

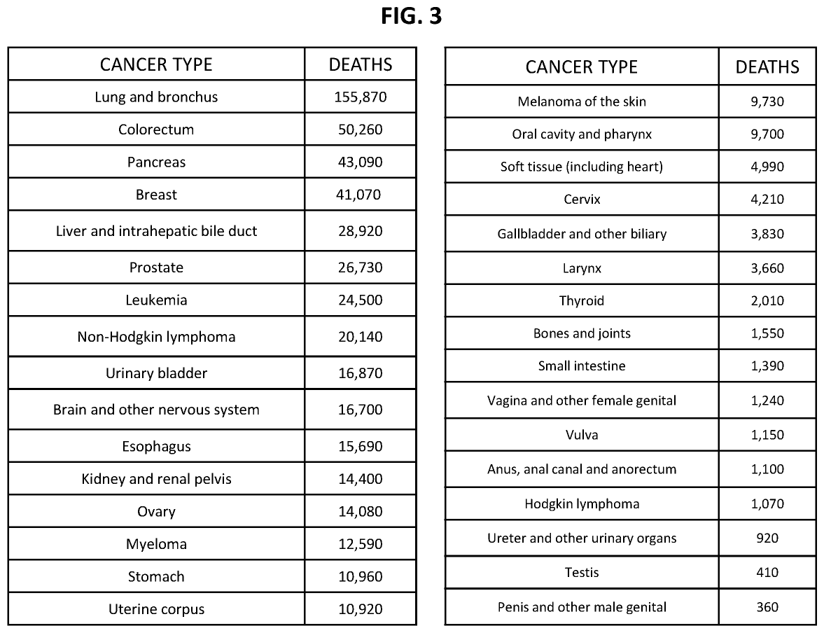

FIG. 4 is a diagram showing an informatics pipeline for identifying non-target antigens.

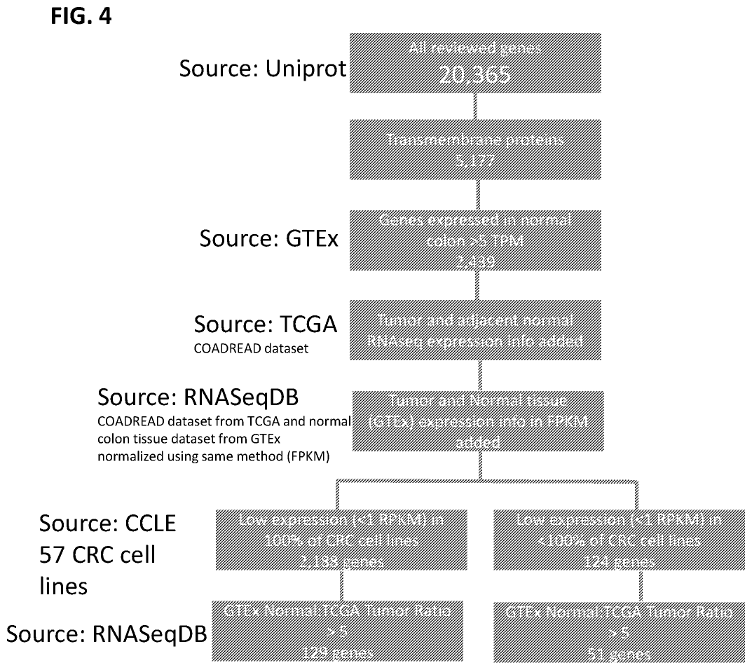

FIG. 5 is a diagram showing the intron and exon domain structure of TGFBR2, including a polyadenine tract prone (SEQ ID NOs: 13 and 14) to expansion in high microsatellite instability colorectal cancers.

### DETAILED DESCRIPTION

Provided herein are compositions and methods for treating cancers using immune cells comprising a two receptor system responsive to differential expression of a non-target antigen in cancer cells and normal tissue. The two-receptor system is expressed in immune cells, for example immune cells used in adoptive cell therapy, and targets activity of these immune cells to cancer cells with low or no expression of a non-target antigen. In this two receptor system, the first receptor (an activator, sometimes referred to herein as an A module) activates, or promote activation of the immune cells, while the second receptor (a blocker, or inhibitor, sometimes referred to herein as a B module) acts to inhibit activation of the immune cells by the first receptor. Each receptor contains a ligand-binding domain (LBD) that binds a specific ligand. Signals from the two receptors upon ligand binding are integrated by the immune cell in a cellular integrator system. Differential expression of ligands for the first and second receptors in cancer and wild type cells (sometimes referred to herein as normal or healthy cells) mediates activation of immune cells by target cancer cells that express the first activator ligand but not the second inhibitory ligand, or express the second inhibitory ligand at a level below that seen in wild type cells.

In particular embodiments of the compositions and methods provided herein, immune cells comprising the two receptor system described herein are used to treat CEA cell adhesion molecule 5 (CEA) positive cancers. This includes CEA positive cancers of the gastro-intestinal (GI) tract. In the case of CEA-positive cancers, the target antigen of the activator receptor is CEA, or a peptide antigen thereof, in a complex with a major histocompatibility complex class I (MHC-I). CEA is predominantly expressed in normal adult in GI tissues as a surface protein that can be cleaved from the membrane and released in soluble form. Because of its selective expression in GI tumors, it has long been considered an attractive tumor-specific antigen that could mediate selective killing of GI tumors if CEA positive cancer cells could be specifically targeted with an appropriate therapeutic. However, normal CEA expression in non-cancer (non-target) cells has prevented the effective use of CEA for targeted therapies such as adoptive cell therapies. Several therapeutics directed against CEA have been tested in the clinic and were found to induce colitis as a dose-limiting toxicity (DLT). In 2011, a clinical study with a murine TCR directed against a CEA peptide complexed with HLA-A*02 (i.e., a pMHC) was stopped in a Phase 1 study (n=3) because of localized toxicity to the colon (Parkhurst et al. Molecular Therapy 2011 19(3): P620-626; Parkhurst et al. Clin Cancer Res. 2009 Jan. 1; 15(1): 169-180). DLT occurred at a remarkably low dose of 2-4E8 cells/patient. By pairing an activator receptor with an inhibitor receptor, the methods provided herein increase the specificity of adoptive cell therapies and decrease harmful effects associated with these therapies, such as dose-limited toxicity.

In some embodiments, the ligand for the activator is a CEA peptide complexed with MHC class I, for example an MHC complex comprising an HLA-A*02. In the methods described herein, this CEA targeted activator receptor is paired with an inhibitory receptor, which increases the safety window of the activator by blocking its cytolytic effect on normal CEA-positive tissues. Without wishing to be bound by theory, these tissues are thought to be mostly in the gastrointestinal tract. However, the activator receptor still directs the targeted killing of tumor cells by immune cells comprising the two-receptor system, as the tumor cells do not express the ligand for the inhibitor, or blocker, receptor. The target for the second, inhibitory receptor is expressed by gastrointestinal (GI) tissues but not by cancer cells, and the inhibitory receptor recognizes this target as an inhibitory stimulus. An exemplary target for the second inhibitory receptor is expressed on the surface of normal GI epithelial cells, and has no expression in GI tumor cells, or sufficiently low expression that the non-target antigen present on the cancer cells does not sufficiently activate the second, inhibitory receptor to the level required for inhibition of immune cell activity.

Without wishing to be bound by theory, it is thought that immune cell inputs for a given activator/inhibitor receptor pair are determined by a simple ratio of target to non-target antigen densities on a particular cell. When both the activator and inhibitor receptor are activated, for example when an immune cell expressing the receptor pair comes in contact with a wild type cell expressing both antigens, activation of both receptors reduces cell surface expression of the activator receptor, but not the inhibitor receptor. Removal of the activator receptor from the surface of the immune cell locally desensitizes the immune cell, and reversibly raises its activation threshold. This leads to inhibition of immune cell activation by the inhibitor receptor. Alternatively, or in addition, cross-talk between the activator and inhibitor receptors may also affect the activation threshold of the immune cell. Thus, use of the activator and receptor pairs of the disclosure can affect baseline activation of immune cells used in adoptive cell therapies, without affecting the sensitivity of these cells for a target antigen such as CEA. A further advantage is that both the activator and inhibitor ligands must be present in cis, on the same cell, to inhibit immune cell activation.

Exemplary targets of the inhibitory receptor include, but are not limited to, bestrophin-2 (BEST2), bestrophin-4 (BEST4), scavenger receptor class A member 5 (SCARA5), ephrin type-A receptor 7 (EPHA7), and transforming growth factor beta receptor 2 (TGFBR2). Each of these is accessible to antibodies, and can be used as a B module target for a cellular integrator designed to safely treat GI cancer patients with engineered T cells activated by an activator receptor such as a CEA or CEA pMHC responsive activator receptor.

In those embodiments where the activator ligand is a CEA ligand, the compositions and methods of the disclosure can reduce or eliminate DLT caused by expression of CEA on normal GI tissue. Without wishing to be bound by theory, it is thought that expression of CEA, while limited, is sufficiently high in the GI tract to induce adverse events of a severity that has prevented further advancement of CEA as a SEQ target for adoptive cell therapy or immunotherapy in the clinic. The disclosure provides methods of targeting CEA in cancer cells to treat CEA positive cancers using adoptive cell therapies by adding a second inhibitory receptor that blocks activation of the adoptive immune cells in the presence of a second ligand (a ligand other than CEA). Using the compositions and methods described herein, tumor cells that express CEA are attacked by the adoptive immune cells expressing the two receptors because these tumor cells express only the activator ligand, CEA, and do not express sufficient levels of the second ligand to block adoptive immune cell activation. In contrast, normal cells that express CEA plus the blocking ligand are protected from the adoptive immune cells. The inhibitory receptor prevents activation of immune cells by the CEA-targeted activator receptor. This dual-targeting approach creates the therapeutic window that will allow a CEA-directed cell therapy to be dosed safely and effectively in CEA-positive cancer patients.

The disclosure provides methods and compositions that allow the use of potent CEA CARs and TCRs that induce on-target toxicity, and renders these CEA targeted receptors useful as a therapeutic by mitigating their toxicity. None of the existing therapeutics that have been tested in the clinic, including cell and large-molecule therapies, provide a mechanism to protect normal CEA-positive tissues.

### Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below. Additional definitions are set forth throughout this disclosure.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

As used herein, the term “isolated” means material that is substantially or essentially free from components that normally accompany it in its native state. In particular embodiments, the term “obtained” or “derived” is used synonymously with isolated.

The terms “subject,” “patient” and “individual” are used interchangeably herein to refer to a vertebrate, preferably a mammal, more preferably a human. Tissues, cells, and their progeny of a biological entity obtained in vivo or cultured in vitro are also encompassed. A “subject,” “patient” or “individual” as used herein, includes any animal that exhibits pain that can be treated with the vectors, compositions, and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

As used herein “treatment” or “treating,” includes any beneficial or desirable effect, and may include even minimal improvement in symptoms. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of a symptom of disease. It also refers to delaying the onset or recurrence of a disease or condition or delaying the occurrence or recurrence of the symptoms of a disease. As used herein, “prevention” and similar words also includes reducing the intensity, effect, symptoms and/or burden of disease prior to onset or recurrence.

As used herein, the term “amount” refers to “an amount effective” or “an effective amount” of a virus to achieve a beneficial or desired prophylactic or therapeutic result, including clinical results.

A “therapeutically effective amount” of a virus or cell may vary according to factors such as the disease state, age, sex, and weight of the individual, and the ability of the virus or cell to elicit a desired response in the individual. A therapeutically effective amount is also one in which any toxic or detrimental effects of the virus or cell are outweighed by the therapeutically beneficial effects. The term “therapeutically effective amount” includes an amount that is effective to “treat” a subject (e.g., a patient).

An “increased” or “enhanced” amount of a physiological response, e.g., electrophysiological activity or cellular activity, is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated cell.

A “decrease” or “reduced” amount of a physiological response, e.g., electrophysiological activity or cellular activity, is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated cell.

By “maintain,” or “preserve,” or “maintenance,” or “no change,” or “no substantial change,” or “no substantial decrease” refers generally to a physiological response that is comparable to a response caused by either vehicle, or a control molecule/composition. A comparable response is one that is not significantly different or measurable different from the reference response.

In general, “sequence identity” or “sequence homology” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values therebetween. Typically, the percent identities between a disclosed sequence and a claimed sequence are at least 80%, at least 85%, at least 90%, at least 95%, or at least 98%.

The term “exogenous” is used herein to refer to any molecule, including nucleic acids, protein or peptides, small molecular compounds, and the like that originate from outside the organism. In contrast, the term “endogenous” refers to any molecule that originates from inside the organism (i.e., naturally produced by the organism).

The term “MOI” is used herein to refer to multiplicity of infection, which is the ratio of agents (e.g. viral particles) to infection targets (e.g. cells).

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%.

As used herein, a “target cell” refers to cell that is targeted by an adoptive cell therapy. For example, a target cell can be cancer cell, which can be killed by the transplanted T cells of the adoptive cell therapy. Target cells of the disclosure express a target antigen, as described herein, and do not express a non-target antigen.

As used herein, a “non-target cell” refers to cell that is not targeted by an adoptive cell therapy. For example, in an adoptive cell targeting cancer cells, normal, healthy, non-cancerous cells are non-target cells. Some, or all, non-target cells in a subject may express both the target antigen and the non-target antigen. Non-target cells in a subject may express the non-target antigen irrespective of whether or not these cells also express the target antigen.

As used herein, a “target antigen refers to an antigen expressed by a target cell, such as a cancer cell. Expression of target antigen is not limited to target cells. Target antigens may be expressed by both cancer cells and normal, non-cancer cells in a subject.

As used herein, a “non-target antigen” refers to an antigen that is expressed by normal, non-cancer cells and is not expressed in cancer cells. This difference in expression allows the inhibitory receptor to inhibit immune cell activation in the presence of non-target cells, but not in the presence of target cells.

As used herein, “affinity” refers to strength of binding of a ligand to a single ligand binding site on a receptor, for example an antigen for the antigen binding domain of any of the receptors described herein. Ligand binding domains can have a weaker interaction (low affinity) with their ligand, or a stronger interaction (high affinity).

Kd, or dissociation constant, is a type of equilibrium constant that measures the propensity of a larger object to separate reversibly into smaller components, such as, for example, when a macromolecular complex comprising receptor and its cognate ligand separates into the ligand and the receptor. When the Kd is high, it means that a high concentration of ligand is need to occupy the receptor, and the affinity of the receptor for the ligand is low. Conversely, a low Kd means that the ligand has a high affinity for the receptor.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

### Activator Receptors

The disclosure provides a first receptor, comprising a first extracellular ligand binding domain specific to a target antigen comprising a cancer cell-specific antigen, or a peptide antigen thereof in a complex with a major histocompatibility complex class I (MHC-I). The first receptor is an activator receptor, and mediates activation of an immune cell expressing the first receptor upon binding of the target antigen by the extracellular ligand binding domain of the first receptor. In some embodiments, the first receptor is a chimeric antigen receptor (CAR). In some embodiments, the first receptor is a T cell receptor (TCR).

In some embodiments, the first receptor is humanized. As used herein, “humanized” refers to the replacement of a sequence or a subsequence in a transgene that has been isolated or derived from a non-human species with a homologous, or functionally equivalent, human sequence. For example, a humanized antibody can be created by grafting mouse CDRs into human framework sequences, followed by back substitution of certain human framework residues for the corresponding mouse residues from the source antibody.

### Activator Targets

In some embodiments, the target antigen for the first receptor is a cancer cell specific antigen. Any cell surface molecule expressed by the target cancer cells may be a suitable target antigen for the first receptor ligand binding domain. For example, a cell adhesion molecule, a cell-cell signaling molecule, an extracellular domain, a molecule involved in chemotaxis, a glycoprotein, a G protein-coupled receptor, a transmembrane, a receptor for a neurotransmitter or a voltage gated ion channel can be used as a target antigen.

In some embodiments, the target antigen is a peptide antigen of a cancer cell-specific antigen in a complex with a major histocompatibility complex class I (MHC-I). Any molecule expressed by the target cancer cells and presented by the major histocompatibility complex class I (MHC-I) on the cancer cell surface as a peptide antigen (pMHC) may be a suitable target antigen for the first receptor extracellular ligand binding domain.

In some embodiments, the cancer cell-specific antigen is CEA cell adhesion molecule 5 (CEA), or a peptide antigen thereof in a complex with a major histocompatibility complex class I (MHC-I).

The major histocompatibility complex class I (MHC-I) is a protein complex that displays antigens to cells of the immune system, triggering an immune response. The Human Leukocyte Antigens (HLAs) corresponding to MHC-I are HLA-A, HLA-B and HLA-C.

Cancer cell-specific pMHC antigens comprising any of HLA-A, HLA-B or HLA-C, HLA-E, HLA-F and HLA-G are envisaged as within the scope of the disclosure. In some embodiments, the cancer cell-specific antigen comprises HLA-A. HLA-A receptors are heterodimers comprising a heavy a chain and smaller R chain. The a chain is encoded by a variant of HLA-A, while the R chain (β2-microglobulin) is an invariant. There are several thousand variant HLA-A genes, all of which fall within the scope of the instant disclosure. In some embodiments, the MHC-I comprises a human leukocyte antigen A*02 allele (HLA-A*02).

In some embodiments, the cancer cell-specific antigen comprises HLA-B. Hundreds of versions (alleles) of the HLA-B gene are known, each of which is given a particular number (such as HLA-B27).

In some embodiments, the cancer cell-specific antigen comprises HLA-C. HLA-C belongs to the HLA class I heavy chain paralogues. This class I molecule is a heterodimer consisting of a heavy chain and a light chain (beta-2 microglobulin). Over one hundred HLA-C alleles are known in the art.

In some embodiments, the cancer cell-specific antigen is a colorectal cancer antigen. In some embodiments, the colorectal cancer antigen comprises CEA.

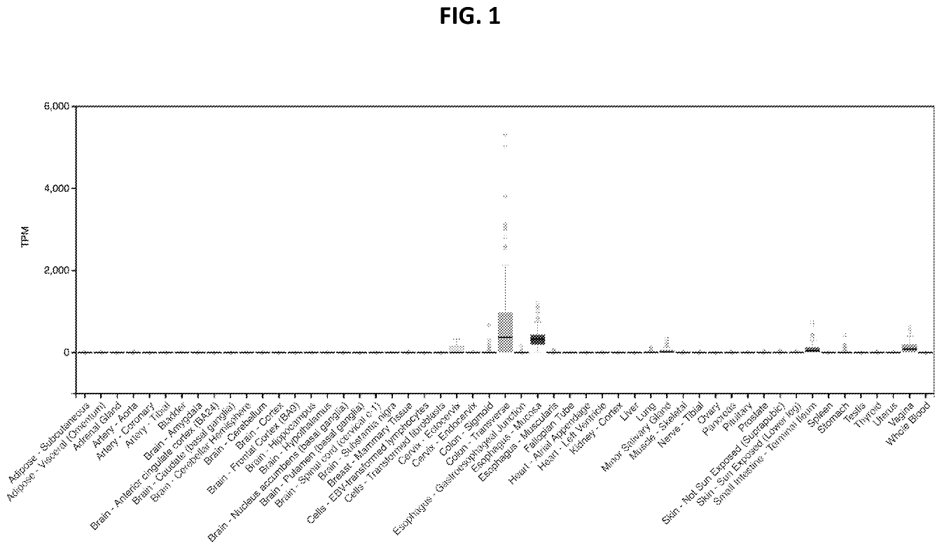

In some embodiments, the cancer cell-specific antigen is CEA cell adhesion molecule 5 (CEA), or a peptide antigen thereof in a complex with a major histocompatibility complex class I (MHC-I). CEA is a 180-kDa glycoprotein tumor-associated protein expressed by a variety of cancer cells. These cancers include adenocarcinomas, colorectal cancers and selected other epithelial cancers, including colorectal adenocarcinomas. However, CEA is also expressed in a variety of normal epithelial cells throughout the gastrointestinal tract, for example in the highly differentiated epithelial cells in the upper third of colonic crypts (see FIG. 1 for CEA expression).

All isoforms of CEA are envisaged as cancer cell-specific antigens of the disclosure. CEA isoform 1 is described in NCBI record number NP_001278413.1, the contents of which are incorporated by reference herein. In some embodiments, CEA comprises an amino acid sequence of:

(SEQ ID NO: 1)

| | |
|---|---|
| 1 | MESPSAPPHR WCIPWQRLLL TASLLTFWNP |
| | PTTAKLTIES TPFNVAEGKE VLLLVHNLPQ |
| 61 | HLFGYSWYKG ERVDGNRQII GYVIGTQQAT |
| | PGPAYSGREI IYPNASLLIQ NIIQNDTGFY |
| 121 | TLHVIKSDLV NEEATGQFRV YPELPKPSIS |
| | SNNSKPVEDK DAVAFTCEPE TQDATYLWWV |
| 181 | NNQSLPVSPR LQLSNGNRTL TLFNVTRNDT |
| | ASYKCETQNP VSARRSDSVI LNVLYGPDAP |
| 241 | TISPLNTSYR SGENLNLSCH AASNPPAQYS |
| | WFVNGTFQQS TQELFIPNIT VNNSGSYTCQ |
| 301 | AHNSDTGLNR TTVTTITVYA EPPKPFITSN |
| | NSNPVEDEDA VALTCEPEIQ NTTYLWWVNN |
| 361 | QSLPVSPRLQ LSNDNRTLTL LSVTRNDVGP |
| | YECGIQNELS VDHSDPVILN VLYGPDDPTI |
| 421 | SPSYTYYRPG VNLSLSCHAA SNPPAQYSWL |
| | IDGNIQQHTQ ELFISNITEK NSGLYTCQAN |
| 481 | NSASGHSRTT VKTITVSAEL PKPSISSNNS |
| | KPVEDKDAVA FTCEPEAQNT TYLWWVNGQS |
| 541 | LPVSPRLQLS NGNRTLTLFN VTRNDARAYV |
| | CGIQNSVSAN RSDPVTLDVL YGPDTPIISP |
| 601 | PDSSYLSGAN LNLSCHSASN PSPQYSWRIN |
| | GIPQQHTQVL FIAKITPNNN GTYACFVSNL |
| 661 | ATGRNNSIVK SITVSASGTS PGLSAGATVG |
| | IMIGVLVGVA LI. |

In some embodiments, CEA comprises a sequence that shares at least 80%, at least 85%, at least 90%, at least 95%, at least 96%, at least 97%, at least 98%, or at least 99% identity to SEQ ID NO: 1. CEA isoform 2 is described in NCBI record number NP_001295327.1, the contents of which are incorporated by reference herein. In some embodiments, CEA comprises an amino acid sequence of:

(SEQ ID NO: 15)

| | |
|---|---|
| 1 | MESPSAPPHR WCIPWQRLLL TASLLTFWNP |
| | PTTAKLTIES TPFNVAEGKE VLLLVHNLPQ |
| 61 | HLFGYSWYKG ERVDGNRQII GYVIGTQQAT |
| | PGPAYSGREI IYPNASLLIQ NIIQNDTGFY |
| 121 | TLHVIKSDLV NEEATGQFRV YPELPKPSIS |
| | SNNSKPVEDK DAVAFTCEPE TQDATYLWWV |
| 181 | NNQSLPVSPR LQLSNGNRTL TLFNVTRNDT |
| | ASYKCETQNP VSARRSDSVI LNVLYGPDAP |
| 241 | TISPLNTSYR SGENLNLSCH AASNPPAQYS |
| | WFVNGTFQQS TQELFIPNIT VNNSGSYTCQ |
| 301 | AHNSDTGLNR TTVTTITVYE PPKPFITSNN |
| | SNPVEDEDAV ALTCEPEIQN TTYLWWVNNQ |
| 361 | SLPVSPRLQL SNDNRTLTLL SVTRNDVGPY |
| | ECGIQNELSV DHSDPVILNV LYGPDDPTIS |
| 421 | PSYTYYRPGV NLSLSCHAAS NPPAQYSWLI |
| | DGNIQQHTQE LFISNITEKN SGLYTCQANN |
| 481 | SASGHSRTTV KTITVSAELP KPSISSNNSK |
| | PVEDKDAVAF TCEPEAQNTT YLWWVNGQSL |
| 541 | PVSPRLQLSN GNRTLTLFNV TRNDARAYVC |
| | GIQNSVSANR SDPVTLDVLY GPDTPIISPP |
| 601 | DSSYLSGANL NLSCHSASNP SPQYSWRING |
| | IPQQHTQVLF IAKITPNNNG TYACFVSNLA |
| 661 | TGRNNSIVKS ITVSASGTSP GLSAGATVGI |
| | MIGVLVGVAL I. |

In some embodiments, CEA comprises a sequence that shares at least 80%, at least 85%, at least 90%, at least 95%, at least 96%, at least 97%, at least 98%, or at least 99% identity to SEQ ID NO: 15.

In some embodiments, the cancer cell-specific antigen is a peptide antigen derived from CEA. In some embodiments, the peptide antigen is comprises a sequence having at least 80%, at least 85%, at least 90%, at least 95%, at least 96%, at least 97%, at least 98%, or at least 99% identity to a subsequence of SEQ ID NO: 1. In some embodiments, the peptide antigen comprises a sequence identical to a subsequence of SEQ ID NO: 1. Exemplary CEA peptide antigens include amino acids 691-699 of SEQ ID NO: 1 (IMIGVLVGV), amino acids 605-613 of SEQ ID NO: 1 (YLSGANLNL), and amino acids 694-702 of SEQ ID NO: 1 (GVLVGVALI). In some embodiments the CEA peptide antigen comprises, or consists essentially of, amino acids 691-699 of SEQ ID NO: 1 (IMIGVLVGV). In some embodiments, the peptide antigen is comprises a sequence having at least 80%, at least 85%, at least 90%, at least 95%, at least 96%, at least 97%, at least 98%, or at least 99% identity to a subsequence of SEQ ID NO: 15. In some embodiments, the peptide antigen comprises a sequence identical to a subsequence of SEQ ID NO: 15. In some embodiments, the CEA peptide antigen is complexed with MHC-I. In some embodiments, the MHC-I comprises a human leukocyte antigen A*02 allele (HLA-A*02).

### Extracellular Ligand Binding Domain

The disclosure provides a first receptor, comprising a first extracellular ligand binding domain specific to a target antigen. In some embodiments, the target antigen comprises a cancer cell-specific antigen.

In some embodiments, the cancer cell-specific antigen is CEA or a CEA-derived peptide antigen complexed with MHC-I, and the ligand binding domain of the first receptor recognizes and binds to the CEA antigen.

Any type of ligand binding domain that can regulate the activity of a receptor in a ligand dependent manner is envisaged as within the scope of the instant disclosure. In some embodiments, the ligand binding domain is an antigen binding domain. Exemplary antigen binding domains include, inter alia, ScFv, SdAb, Vβ-only domains, and TCR antigen binding domains derived from the TCR α and β chain variable domains.

Any type of antigen binding domain is envisaged as within the scope of the instant disclosure.

For example, the first extracellular ligand binding domain may be part of a contiguous polypeptide chain including, for example, a Vβ-only domain, a single domain antibody fragment (sdAb) or heavy chain antibodies HCAb, a single chain antibody (scFv) derived from a murine, humanized or human antibodies (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some aspects, the first extracellular ligand binding domain comprises an antigen binding domain that comprises an antibody fragment. In further aspects, the first extracellular ligand binding domain comprises an antibody fragment that comprises a scFv or an sdAb.

The term “antibody,” as used herein, refers to a protein, or polypeptide sequences derived from an immunoglobulin molecule, which specifically binds to an antigen. Antibodies can be intact immunoglobulins of polyclonal or monoclonal origin, or fragments thereof and can be derived from natural or from recombinant sources.

The terms “antibody fragment” or “antibody binding domain” refer to at least one portion of an antibody, or recombinant variants thereof, that contains the antigen binding domain, i.e., an antigenic determining variable region of an intact antibody, that is sufficient to confer recognition and specific binding of the antibody fragment to a target, such as an antigen and its defined epitope. Examples of antibody fragments include, but are not limited to, Fab, Fab′, F(ab′)2, and Fv fragments, single-chain (sc)Fv (“scFv”) antibody fragments, linear antibodies, single domain antibodies (abbreviated “sdAb”) (either VL or VH), camelid VHH domains, and multi-specific antibodies formed from antibody fragments.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived.

“Heavy chain variable region” or “VH” (or, in the case of single domain antibodies, e.g., nanobodies, “VHH”) with regard to an antibody refers to the fragment of the heavy chain that contains three CDRs interposed between flanking stretches known as framework regions, these framework regions are generally more highly conserved than the CDRs and form a scaffold to support the CDRs.

Unless specified, as used herein a scFv may have the VL and VH variable regions in either order, e.g., with respect to the N-terminal and C-terminal ends of the polypeptide, the scFv may comprise VL-linker-VH or may comprise VH-linker-VL.

The term “antibody light chain,” refers to the smaller of the two types of polypeptide chains present in antibody molecules in their naturally occurring conformations. Kappa (“K”) and lambda (“λ”) light chains refer to the two major antibody light chain isotypes.

The term “recombinant antibody” refers to an antibody that is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “VP domain”, “Vβ-only domain”, “P chain variable domain” or “single variable domain TCR (svd-TCR)” refers to an antigen binding domain that consists essentially of a single T Cell Receptor (TCR) beta variable domain that specifically binds to an antigen in the absence of a second TCR variable domain. The Vβ-only domain engages antigen using complementarity-determining regions (CDRs). Each Vβ-only domain contains three complement determining regions (CDR1, CDR2, and CDR3). Additional elements may be combined provided that the VP domain is configured to bind the epitope in the absence of a second TCR variable domain.

In some embodiments, the extracellular ligand binding domain of the first receptor comprises an antibody fragment, a single chain Fv antibody fragment (ScFv), or a chain variable domain (Vβ).

In some embodiments, the extracellular ligand binding domain of the first receptor comprises a TCR α chain variable domain and a TCR β chain variable domain.

In some embodiments, the first extracellular ligand binding domain comprises a TCR ligand binding domain that binds to a CEA antigen. In some embodiments, the CEA antigen is complexed with MHC-I, and the MHC-I comprises an HLA-A*02 allele. Exemplary TCR antigen binding domains that bind to and recognize CEA MHC-I HLA-A*02 antigens are described in Parkhurst et al. Molecular Therapy 2011 19(3): P620-626, the contents of which are incorporated herein by reference. An exemplary TCR extracellular ligand binding domain that recognizes amino acids 691-699 of SEQ ID NO: 1 (IMIGVLVGV) complexed with HLA-A*02 MHC-I comprises a TCR alpha domain of TRAV8-1*01 and TRAJ6*O1, and a TCR beta domain of TRBV26*O1, TRBD1*O1, TRBJ2-7*01 and TRBC2.

Exemplary CDRs for that recognize a CEA MHC-I HLA-A*02 antigen comprising IMIGVLVGV are shown in Table 1 below.

TABLE 1
CDRs for MHC-I HLA-A*02 + CEA (IMIGVLVGV)

| | A-CDR1 | A-CDR2 | A-CDR3 | B-CDR1 | B-CDR2 | B-CDR3 | Note |
|---|---|---|---|---|---|---|---|
| 1 | TSITA | IRSNER | ATDLTS | KGHPV | FONQE | ASSLGLGDYEQ | “WT” |
| | (SEQ ID | (SEQ ID | GGNYK | (SEQ ID | V (SEQ | (SEQ ID NO: 11) | |
| | NO: 3) | NO: 4) | (SEQ ID | NO: 9) | ID NO: | | |
| | | | NO: 5) | | 10) | | |
| 2 | | | | | | ASSLGTGDYEQ | BV117T |
| | | | | | | (SEQ ID NO: 12) | |
| 3 | | | ATDFTS | | | ASSLGLGDYEQ | AV-L110F |
| | | | GGNYK | | | (SEQ ID NO: 11) | |
| | | | (SEQ ID | | | | |
| | | | NO: 6) | | | | |
| 4 | | | | | | ASSLGTGDYEQ | AV- |
| | | | | | | (SEQ ID NO: 12) | L110F/ |
| | | | | | | | BV117T |
| 5 | | | ATDLT | | | ASSLGLGDYEQ | AV-S112T |
| | | | TGGNY | | | (SEQ ID NO: 11) | |
| | | | K (SEQ | | | | |
| | | | ID NO: | | | | |
| | | | 7) | | | | |
| 6 | | | | | | ASSLGTGDYEQ | AV-S112T/ |
| | | | | | | (SEQ ID NO: 12) | BV117T |
| 7 | | | ATDFTT | | | ASSLGLGDYEQ | AV- |
| | | | GGNYK | | | (SEQ ID NO: 11) | L110FS112T |
| | | | (SEQ ID | | | | |
| | | | NO: 8) | | | | |
| 8 | | | | | | ASSLGTGDYEQ | AV- |
| | | | | | | (SEQ ID NO: 12) | L110FS112T/ |
| | | | | | | | BV117T |

In some embodiments, the first extracellular ligand binding domain comprises complement determining regions (CDRs) selected from SEQ ID NOs: 3-12 or sequences having at least 85% or at least 95% identity thereto.

In some embodiments, the ligand binding domain of the first receptor comprises a TCR ligand binding domain. In some embodiments, the TCR α chain variable domain comprises a CDR-1 of TSITA (SEQ ID NO: 3), a CDR-2 of IRSNER (SEQ ID NO: 4) and a CDR-3 comprising ATDLTSGGNYK (SEQ ID NO: 5), ATDFTSGGNYK (SEQ ID NO: 6), ATDLTTGGNYK (SEQ ID NO: 7) or ATDFTTGGNYK (SEQ ID NO: 8); and the TCR β chain variable domain comprises a CDR-1 of KGHPV (SEQ ID NO: 9), a CDR-2 of FQNQEV (SEQ ID NO: 10), and a CDR-3 of ASSLGLGDYEQ (SEQ ID NO: 11) or ASSLGTGDYEQ (SEQ ID NO: 12), or sequences having at least 85% or at least 95% identity thereto. In some embodiments, the TCR α chain variable domain comprises a CDR-1 of SEQ ID NO: 9, a CDR-2 of SEQ ID NO: 10 and a CDR-3 of SEQ ID NO: 11 or SEQ ID NO: 12; and the TCR β chain variable domain comprises a CDR-1 of SEQ ID NO: 3, a CDR-2 of SEQ ID NO: 4 and a CDR-3 comprising SEQ ID NO: 5, SEQ ID NO: 6, SEQ ID NO: 7 or SEQ ID NO: 8, or sequences having at least 85% or at least 95% identity thereto.

Exemplary TCR alpha and beta chains comprising the CDRs from Table 1 are shown in Table 2 below. CDRs are underlined in the sequences in Table 2. In Table 2, the TCR alpha and TCR beta chains are separated by a P2A self-cleaving peptide (ATNFSLLKQAGDVEENPGP (SEQ ID NO: 32)) and a GSG linker.

TABLE 2
MHC-I HLA-A*02 + CEA (IMIGVLVGV) TCR sequences

| Construct | Amino Acid Sequence | DNA Sequence |
|---|---|---|
| CT 548: pLenti | MHSLLGLLMVSLWLQLTRVNSQLAEENPWALSVHEGESVTVNCSYKTSIT | (SEQ ID NO: 97) |
| 1 CEA TCR | ALQWYRQKSGEGPAQLILIRSNEREKRNGRLRATLDTSSQSSSLSITATRCE | |
| TRAV8-1*01 | DTAVYFCATDLTSGGNYKPTFGKGTSLVVHPDIQNPEPAVYQLKDPRSQD | |
| 118P & 119T | STLCLFTDFDSQINVPKTMESGTFITDKTVLDMKAMDSKSNGAIAWSNQT | |
| with murine | SFTCQDIFKETNATYPSSDVPCDATLTEKSFETDMNLNFQNLSVMGLRILLL | |
| constant | KVAGFNLLMTLRLWSSGSGATNFSLLKQAGDVEENPGPMATRLLCYTVLC | |
| region | LLGARILNSKVIQTPRYLVKGQGQKAKMRCIPEKGHPVVFWYQQNKNNE | |
| | FKFLINFQNQEVLQQIDMTEKRFSAECPSNSPCSLEIQSSEAGDSALYLCAS | |
| | SLGLGDYEQYFGPGTRLTVLEDLRNVTPPKVSLFEPSKAEIANKQKATLVCL | |
| | ARGFFPDHVELSWWVNGKEVHSGVSTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| | (SEQ ID NO: 16) | |
| CT 549: pLenti | MHSLLGLLMVSLWLQLTRVNSQLAEENPWALSVHEGESVTVNCSYKTSITA | (SEQ ID NO: 98) |
| 1 CEA TCR | LQWYRQKSGEGPAQLILIRSNEREKRNGRLRATLDTSSQSSSLSITATRCE | |
| TRAV8-1*01 | DTAVYFCATDLTSGGNYKPTFGKGTSLVVHPDIQNPEPAVYQLKDPRSQD | |
| 118P & 119T | STLCLFTDFDSQINVPKTMESGTFITDKTVLDMKAMDSKSNGAIAWSNQTS | |
| TRBV26*01 | FTCQDIFKETNATYPSSDVPCDATLTEKSFETDMNLNFQNLSVMGLRILLL | |
| L117T with | KVAGFNLLMTLRLWSSGSGATNFSLLKQAGDVEENPGPMATRLLCYTVLC | |
| murine | LLGARILNSKVIQTPRYLVKGQGQKAKMRCIPEKGHPVVFWYQQNKNNE | |
| constant | FKFLINFQNQEVLQQIDMTEKRFSAECPSNSPCSLEIQSSEAGDSALYLCAS | |
| region | SLGTGDYEQYFGPGTRLTVLEDLRNVTPPKVSLFEPSKAEIANKQKATLVCL | |
| | ARGFFPDHVELSWWVNGKEVHSGVSTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| | (SEQ ID NO: 17) | |
| CT 550: pLenti | MHSLLGLLMVSLWLQLTRVNSQLAEENPWALSVHEGESVTVNCSYKTSIT | (SEQ ID NO: 99) |
| 1 CEA TCR | ALQWYRQKSGEGPAQLILIRSNEREKRNGRLRATLDTSSQSSSLSITATRCE | |
| TRAV8-1*01 | DTAVYFCATDLTSGGNYKPTFGKGTSLVVHPNIQNPEPAVYQLKDPRSQD | |
| 118P & 119T | STLCLFTDFDSQINVPKTMESGTFITDKCVLDMKAMDSKSNGAIAWSNQT | |
| with murine | SFTCQDIFKETNATYPSSDVPCDATLTEKSFETDMNLNFQNLLVIVLRILLLK | |
| constant | VAGFNLLMTLRLWSSGSGATNFSLLKQAGDVEENPGPMATRLLCYTVLCL | |
| region | LGARILNSKVIQTPRYLVKGQGQKAKMRCIPEKGHPVVFWYQQNKNNEF | |
| | KFLINFQNQEVLQQIDMTEKRFSAECPSNSPCSLEIQSSEAGDSALYLCASS | |
| | LGLGDYEQYFGPGTRLTVLEDLRNVTPPKVSLFEPSKAEIANKQKATLVCLA | |
| | RGFFPDHVELSWWVNGKEVHSGVCTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| | (SEQ ID NO: 18) | |
| CT 551: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 100) |
| TCR TRAV8- | DTAVYFCATDFTSGGNYKPTFGKGTSLVVHPDIQNPEPAVYQLKDPRSQD | |
| 1*01 L110F | STLCLFTDFDSQINVPKTMESGTFITDKTVLDMKAMDSKSNGAIAWSNQT | |
| 118P & 119T | SFTCQDIFKETNATYPSSDVPCDATLTEKSFETDMNLNFQNLSVMGLRILLL | |
| with murine | KVAGFNLLMTLRLWSSGSGATNFSLLKQAGDVEENPGPMATRLLCYTVLC | |
| constant | LLGARILNSKVIQTPRYLVKGQGQKAKMRCIPEKGHPVVFWYQQNKNNE | |
| region | FKFLINFQNQEVLQQIDMTEKRFSAECPSNSPCSLEIQSSEAGDSALYLCAS | |
| | SLGLGDYEQYFGPGTRLTVLEDLRNVTPPKVSLFEPSKAEIANKQKATLVCL | |
| | ARGFFPDHVELSWWVNGKEVHSGVSTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| | (SEQ ID NO: 19) | |
| CT 552: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 101) |
| TCR TRAV8- | DTAVYFCATDLTTGGNYKPTFGKGTSLVVHPDIQNPEPAVYQLKDPRSQD | |
| 1*01 S112T | STLCLFTDFDSQINVPKTMESGTFITDKTVLDMKAMDSKSNGAIAWSNQT | |
| 118P & 119T | SFTCQDIFKETNATYPSSDVPCDATLTEKSFETDMNLNFQNLSVMGLRILLL | |
| with murine | KVAGFNLLMTLRLWSSGSGATNFSLLKQAGDVEENPGPMATRLLCYTVLC | |
| constant | LLGARILNSKVIQTPRYLVKGQGQKAKMRCIPEKGHPVVFWYQQNKNNE | |
| region | FKFLINFQNQEVLQQIDMTEKRFSAECPSNSPCSLEIQSSEAGDSALYLCAS | |
| | SLGLGDYEQYFGPGTRLTVLEDLRNVTPPKVSLFEPSKAEIANKQKATLVCL | |
| | ARGFFPDHVELSWWVNGKEVHSGVSTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| | (SEQ ID NO: 20) | |
| CT 553: | MHSLLGLLMVSLWLQLTRVNSQLAEENPWALSVHEGESVTVNCSYKTSITA | (SEQ ID NO: |
| pLenti 1 CEA | LQWYRQKSGEGPAQLILIRSNEREKRNGRLRATLDTSSQSSSLSITATRCE | 102) |
| TCR TRAV8- | DTAVYFCATDFTTGGNYKPTFGKGTSLVVHPDIQNPEPAVYQLKDPRSQD | |
| 1*01 L110F | STLCLFTDFDSQINVPKTMESGTFITDKTVLDMKAMDSKSNGAIAWSNQT | |
| S112T 118P & | SFTCQDIFKETNATYPSSDVPCDATLTEKSFETDMNLNFQNLSVMGLRILL | |
| 119T with | LKVAGFNLLMTLRLWSSGSGATNFSLLKQAGDVEENPGPMATRLLCYTVLC | |
| murine | LLGARILNSKVIQTPRYLVKGQGQKAKMRCIPEKGHPVVFWYQQNKNNEF | |
| constant | KFLINFQNQEVLQQIDMTEKRFSAECPSNSPCSLEIQSSEAGDSALYLCAS | |
| region | SLGLGDYEQYFGPGTRLTVLEDLRNVTPPKVSLFEPSKAEIANKQKATLVC | |
| | LARGFFPDHVELSWWVNGKEVHSGVSTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| | (SEQ ID NO: 21) | |
| CT 554: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 103) |
| TCR TRAV8- | DTAVYFCATDFTSGGNYKPTFGKGTSLVVHPDIQNPEPAVYQLKDPRSQD | |
| 1*01 L110F | STLCLFTDFDSQINVPKTMESGTFITDKTVLDMKAMDSKSNGAIAWSNQT | |
| 118P & 119T | SFTCQDIFKETNATYPSSDVPCDATLTEKSFETDMNLNFQNLSVMGLRILLL | |
| TRBV26*01 | KVAGFNLLMTLRLWSSGSGATNFSLLKQAGDVEENPGPMATRLLCYTVLC | |
| L117T with | LLGARILNSKVIQTPRYLVKGQGQKAKMRCIPEKGHPVVFWYQQNKNNE | |
| murine | FKFLINFQNQEVLQQIDMTEKRFSAECPSNSPCSLEIQSSEAGDSALYLCAS | |
| constant | SLGTGDYEQYFGPGTRLTVLEDLRNVTPPKVSLFEPSKAEIANKQKATLVCL | |
| region | ARGFFPDHVELSWWVNGKEVHSGVSTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| | (SEQ ID NO: 22) | |
| CT 555: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 104) |
| TCR TRAV8- | DTAVYFCATDLTTGGNYKPTFGKGTSLVVHPDIQNPEPAVYQLKDPRSQD | |
| 1*01 S112T | STLCLFTDFDSQINVPKTMESGTFITDKTVLDMKAMDSKSNGAIAWSNQT | |
| 118P & 119T | SFTCQDIFKETNATYPSSDVPCDATLTEKSFETDMNLNFQNLSVMGLRILLL | |
| TRBV26*01 | KVAGFNLLMTLRLWSSGSGATNFSLLKQAGDVEENPGPMATRLLCYTVLC | |
| L117T with | LLGARILNSKVIQTPRYLVKGQGQKAKMRCIPEKGHPVVFWYQQNKNNE | |
| murine | FKFLINFQNQEVLQQIDMTEKRFSAECPSNSPCSLEIQSSEAGDSALYLCAS | |
| constant | SLGTGDYEQYFGPGTRLTVLEDLRNVTPPKVSLFEPSKAEIANKQKATLVCL | |
| region | ARGFFPDHVELSWWVNGKEVHSGVSTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| | (SEQ ID NO: 23) | |
| CT 556: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 105) |
| TCR TRAV8- | DTAVYFCATDFTTGGNYKPTFGKGTSLVVHPDIQNPEPAVYQLKDPRSQD | |
| 1*01 L110F | STLCLFTDFDSQINVPKTMESGTFITDKTVLDMKAMDSKSNGAIAWSNQT | |
| S112T 118P & | SFTCQDIFKETNATYPSSDVPCDATLTEKSFETDMNLNFQNLSVMGLRILLL | |
| 119T | KVAGFNLLMTLRLWSSGSGATNFSLLKQAGDVEENPGPMATRLLCYTVLC | |
| TRBV26*01 | LLGARILNSKVIQTPRYLVKGQGQKAKMRCIPEKGHPVVFWYQQNKNNE | |
| L117T with | FKFLINFQNQEVLQQIDMTEKRFSAECPSNSPCSLEIQSSEAGDSALYLCAS | |
| murine | SLGTGDYEQYFGPGTRLTVLEDLRNVTPPKVSLFEPSKAEIANKQKATLVCL | |
| constant | ARGFFPDHVELSWWVNGKEVHSGVSTDPQAYKESNYSYCLSSRLRVSATF | |
| region | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| | (SEQ ID NO: 24) | |
| CT 557: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 106) |
| TCR TRAV8- | DTAVYFCATDFTSGGNYKPTFGKGTSLVVHPNIQNPEPAVYQLKDPRSQD | |
| 1*01 L110F | STLCLFTDFDSQINVPKTMESGTFITDKCVLDMKAMDSKSNGAIAWSNQT | |
| 118P & 119T | SFTCQDIFKETNATYPSSDVPCDATLTEKSFETDMNLNFQNLLVIVLRILLLK | |
| with murine | VAGFNLLMTLRLWSSGSGATNFSLLKQAGDVEENPGPMATRLLCYTVLCL | |
| constant | LGARILNSKVIQTPRYLVKGQGQKAKMRCIPEKGHPVVFWYQQNKNNEF | |
| region | KFLINFONQEVLQQIDMTEKRFSAECPSNSPCSLEIQSSEAGDSALYLCASS | |
| | LGLGDYEQYFGPGTRLTVLEDLRNVTPPKVSLFEPSKAEIANKQKATLVCLA | |
| | RGFFPDHVELSWWVNGKEVHSGVCTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| | (SEQ ID NO: 25) | |
| CT 558: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 107) |
| TCR TRAV8- | DTAVYFCATDLTTGGNYKPTFGKGTSLVVHPNIQNPEPAVYQLKDPRSQD | |
| 1*01 S112T | STLCLFTDFDSQINVPKTMESGTFITDKCVLDMKAMDSKSNGAIAWSNQT | |
| 118P & 119T | SFTCQDIFKETNATYPSSDVPCDATLTEKSFETDMNLNFQNLLVIVLRILLLK | |
| with murine | VAGFNLLMTLRLWSSGSGATNFSLLKQAGDVEENPGPMATRLLCYTVLCL | |
| constant | LGARILNSKVIQTPRYLVKGQGQKAKMRCIPEKGHPVVFWYQQNKNNEF | |
| region | KFLINFQNQEVLQQIDMTEKRFSAECPSNSPCSLEIQSSEAGDSALYLCASS | |
| | LGLGDYEQYFGPGTRLTVLEDLRNVTPPKVSLFEPSKAEIANKQKATLVCLA | |
| | RGFFPDHVELSWWVNGKEVHSGVCTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS ** | |
| | (SEQ ID NO: 26) | |
| CT 559: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 108) |
| TCR TRAV8- | DTAVYFCATDFTTGGNYKPTFGKGTSLVVHPNIQNPEPAVYQLKDPRSQD | |
| 1*01 L110F | STLCLFTDFDSQINVPKTMESGTFITDKCVLDMKAMDSKSNGAIAWSNQT | |
| S112T 118P & | SFTCQDIFKETNATYPSSDVPCDATLTEKSFETDMNLNFQNLLVIVLRILLLK | |
| 119T with | VAGFNLLMTLRLWSSGSGATNFSLLKQAGDVEENPGPMATRLLCYTVLCL | |
| murine | LGARILNSKVIQTPRYLVKGQGQKAKMRCIPEKGHPVVFWYQQNKNNEF | |
| constant | KFLINFQNQEVLQQIDMTEKRFSAECPSNSPCSLEIQSSEAGDSALYLCASS | |
| region | LGLGDYEQYFGPGTRLTVLEDLRNVTPPKVSLFEPSKAEIANKQKATLVCLA | |
| | RGFFPDHVELSWWVNGKEVHSGVCTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| | (SEQ ID NO: 27) | |
| CT 560: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 109) |
| TCR TRAV8- | DTAVYFCATDLTSGGNYKPTFGKGTSLVVHPNIQNPEPAVYQLKDPRSQD | |
| 1*01 118P & | STLCLFTDFDSQINVPKTMESGTFITDKCVLDMKAMDSKSNGAIAWSNQT | |
| 119T | SFTCQDIFKETNATYPSSDVPCDATLTEKSFETDMNLNFQNLLVIVLRILLLK | |
| TRBV26*01 | VAGFNLLMTLRLWSSGSGATNFSLLKQAGDVEENPGPMATRLLCYTVLCL | |
| L117T with | LGARILNSKVIQTPRYLVKGQGQKAKMRCIPEKGHPVVFWYQQNKNNEF | |
| murine | KFLINFQNQEVLQQIDMTEKRFSAECPSNSPCSLEIQSSEAGDSALYLCASS | |
| | LGTGDYEQYFGPGTRLTVLEDLRNVTPPKVSLFEPSKAEIANKQKATLVCLA | |
| constant | RGFFPDHVELSWWVNGKEVHSGVCTDPQAYKESNYSYCLSSRLRVSATF | |
| region | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| | (SEQ ID NO: 28) | |
| CT 561: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 110) |
| TCR TRAV8- | DTAVYFCATDFTSGGNYKPTFGKGTSLVVHPNIQNPEPAVYQLKDPRSQD | |
| 1*01 L110F | STLCLFTDFDSQINVPKTMESGTFITDKCVLDMKAMDSKSNGAIAWSNQT | |
| 118P & 119T | SFTCQDIFKETNATYPSSDVPCDATLTEKSFETDMNLNFQNLLVIVLRILLLK | |
| TRBV26*01 | VAGFNLLMTLRLWSSGSGATNFSLLKQAGDVEENPGPMATRLLCYTVLCL | |
| L117T with | LGARILNSKVIQTPRYLVKGQGQKAKMRCIPEKGHPVVFWYQQNKNNEF | |
| murine | KFLINFQNQEVLQQIDMTEKRFSAECPSNSPCSLEIQSSEAGDSALYLCASS | |
| constant | LGTGDYEQYFGPGTRLTVLEDLRNVTPPKVSLFEPSKAEIANKQKATLVCLA | |
| region | RGFFPDHVELSWWVNGKEVHSGVCTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| | (SEQ ID NO: 29) | |
| CT 562: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 111) |
| TCR TRAV8- | DTAVYFCATDLTTGGNYKPTFGKGTSLVVHPNIQNPEPAVYQLKDPRSQD | |
| 1*01 S112T | STLCLFTDFDSQINVPKTMESGTFITDKCVLDMKAMDSKSNGAIAWSNQT | |
| 118P & 119T | SFTCQDIFKETNATYPSSDVPCDATLTEKSFETDMNLNFQNLLVIVLRILLLK | |
| TRBV26*01 | VAGFNLLMTLRLWSSGSGATNFSLLKQAGDVEENPGPMATRLLCYTVLCL | |
| L117T with | LGARILNSKVIQTPRYLVKGQGQKAKMRCIPEKGHPVVFWYQQNKNNEF | |
| murine | KFLINFQNQEVLQQIDMTEKRFSAECPSNSPCSLEIQSSEAGDSALYLCASS | |
| constant | LGTGDYEQYFGPGTRLTVLEDLRNVTPPKVSLFEPSKAEIANKQKATLVCLA | |
| region | RGFFPDHVELSWWVNGKEVHSGVCTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| | (SEQ ID NO: 30) | |
| CT 563: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 112) |
| TCR TRAV8- | DTAVYFCATDFTTGGNYKPTFGKGTSLVVHPNIQNPEPAVYQLKDPRSQD | |
| 1*01 L110F | STLCLFTDFDSQINVPKTMESGTFITDKCVLDMKAMDSKSNGAIAWSNQT | |
| S112T 118P & | SFTCQDIFKETNATYPSSDVPCDATLTEKSFETDMNLNFQNLLVIVLRILLLK | |
| 119T | VAGFNLLMTLRLWSSGSGATNFSLLKQAGDVEENPGPMATRLLCYTVLCL | |
| TRBV26*01 | LGARILNSKVIQTPRYLVKGQGQKAKMRCIPEKGHPVVFWYQQNKNNEF | |
| L117T with | KFLINFQNQEVLQQIDMTEKRFSAECPSNSPCSLEIQSSEAGDSALYLCASS | |
| murine | LGTGDYEQYFGPGTRLTVLEDLRNVTPPKVSLFEPSKAEIANKQKATLVCLA | |
| constant | RGFFPDHVELSWWVNGKEVHSGVCTDPQAYKESNYSYCLSSRLRVSATF | |
| region | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| | (SEQ ID NO: 31) | |
| CT 532: pLenti | MHSLLGLLMVSLWLQLTRVNSQLAEENPWALSVHEGESVTVNCSYKTSIT | (SEQ ID NO: |
| 1 CEA TCR | ALQWYRQKSGEGPAQLILIRSNEREKRNGRLRATLDTSSQSSSLSITATRCE | 113) |
| TRAV8-1*01 | DTAVYFCATDLTSGGNYKFGKGTSLVVHPDIQNPEPAVYQLKDPRSQDST | |
| TRBV26*01 | LCLFTDFDSQINVPKTMESGTFITDKTVLDMKAMDSKSNGAIAWSNQTSF | |
| with regular | TCQDIFKETNATYPSSDVPCDATLTEKSFETDMNLNFQNLSVMGLRILLLK | |
| murine | VAGFNLLMTLRLWSSGSGATNFSLLKQAGDVEENPGPMATRLLCYTVLCL | |
| constant | LGARILNSKVIQTPRYLVKGQGQKAKMRCIPEKGHPVVFWYQQNKNNEF | |
| region | KFLINFQNQEVLQQIDMTEKRFSAECPSNSPCSLEIQSSEAGDSALYLCASS | |
| | LGLGDYEQYFGPGTRLTVLEDLRNVTPPKVSLFEPSKAEIANKQKATLVCLA | |
| | RGFFPDHVELSWWVNGKEVHSGVSTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| | (SEQ ID NO: 36) | |
| CT 533; | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 114) |
| TCR TRAV8- | DTAVYFCATDLTSGGNYKFGKGTSLVVHPDIQNPEPAVYQLKDPRSQDST | |
| 1*01 | LCLFTDFDSQINVPKTMESGTFITDKTVLDMKAMDSKSNGAIAWSNQTSF | |
| TRBV26*01 | TCQDIFKETNATYPSSDVPCDATLTEKSFETDMNLNFQNLSVMGLRILLLK | |
| L117T with | VAGFNLLMTLRLWSSGSGATNFSLLKQAGDVEENPGPMATRLLCYTVLCL | |
| regular | LGARILNSKVIQTPRYLVKGQGQKAKMRCIPEKGHPVVFWYQQNKNNEF | |
| murine | KFLINFQNQEVLQQIDMTEKRFSAECPSNSPCSLEIQSSEAGDSALYLCASS | |
| constant | LGTGDYEQYFGPGTRLTVLEDLRNVTPPKVSLFEPSKAEIANKQKATLVCLA | |
| region | RGFFPDHVELSWWVNGKEVHSGVSTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| | (SEQ ID NO: 37) | |
| CT 534: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 115) |
| TCR TRAV8- | DTAVYFCATDLTSGGNYKFGKGTSLVVHPNIQNPEPAVYQLKDPRSQDST | |
| 1*01 | LCLFTDFDSQINVPKTMESGTFITDKCVLDMKAMDSKSNGAIAWSNQTSF | |
| TRBV26*01 | TCQDIFKETNATYPSSDVPCDATLTEKSFETDMNLNFQNLLVIVLRILLLKVA | |
| murine | GFNLLMTLRLWSSGSGATNFSLLKQAGDVEENPGPMATRLLCYTVLCLLG | |
| constant | ARILNSKVIQTPRYLVKGQGQKAKMRCIPEKGHPVVFWYQQNKNNEFKF | |
| region (no PT) | LINFQNQEVLQQIDMTEKRFSAECPSNSPCSLEIQSSEAGDSALYLCASSLG | |
| | LGDYEQYFGPGTRLTVLEDLRNVTPPKVSLFEPSKAEIANKQKATLVCLARG | |
| | FFPDHVELSWWVNGKEVHSGVCTDPQAYKESNYSYCLSSRLRVSATFWH | |
| | NPRNHFRCQVQFHGLSEEDKWPEGSPKPVTQNISAEAWGRADCGITSAS | |
| | YQQGVLSATILYEILLGKATLYAVLVSTLVVMAMVKRKNS (SEQ ID NO: | |
| | 38) | |
| CT 535: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 116 |
| TCR TRAV8- | DTAVYFCATDFTSGGNYKFGKGTSLVVHPDIQNPEPAVYQLKDPRSQDST | |
| 1*01 L110F | LCLFTDFDSQINVPKTMESGTFITDKTVLDMKAMDSKSNGAIAWSNQTSF | |
| TRBV26*01 | TCQDIFKETNATYPSSDVPCDATLTEKSFETDMNLNFQNLSVMGLRILLLK | |
| with regular | VAGFNLLMTLRLWSSGSGATNFSLLKQAGDVEENPGPMATRLLCYTVLCL | |
| murine | LGARILNSKVIQTPRYLVKGQGQKAKMRCIPEKGHPVVFWYQQNKNNEF | |
| constant | KFLINFQNQEVLQQIDMTEKRFSAECPSNSPCSLEIQSSEAGDSALYLCASS | |
| region | LGLGDYEQYFGPGTRLTVLEDLRNVTPPKVSLFEPSKAEIANKQKATLVCLA | |
| | RGFFPDHVELSWWVNGKEVHSGVSTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS (SEQ ID | |
| | NO: 39) | |
| CT 536: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 117) |
| TCR TRAV8- | DTAVYFCATDLTTGGNYKFGKGTSLVVHPDIQNPEPAVYQLKDPRSQDST | |
| 1*01 S112T | LCLFTDFDSQINVPKTMESGTFITDKTVLDMKAMDSKSNGAIAWSNQTSF | |
| TRBV26*01 | TCQDIFKETNATYPSSDVPCDATLTEKSFETDMNLNFQNLSVMGLRILLLK | |
| with regular | VAGFNLLMTLRLWSSGSGATNFSLLKQAGDVEENPGPMATRLLCYTVLCL | |
| murine | LGARILNSKVIQTPRYLVKGQGQKAKMRCIPEKGHPVVFWYQQNKNNEF | |
| constant | KFLINFQNQEVLQQIDMTEKRFSAECPSNSPCSLEIQSSEAGDSALYLCASS | |
| region | LGLGDYEQYFGPGTRLTVLEDLRNVTPPKVSLFEPSKAEIANKQKATLVCLA | |
| | RGFFPDHVELSWWVNGKEVHSGVSTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS (SEQ ID | |
| | NO: 40) | |
| CT 537: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 118) |
| TCR TRAV8- | DTAVYFCATDFTTGGNYKFGKGTSLVVHPDIQNPEPAVYQLKDPRSQDST | |
| 1*01 L110F & | LCLFTDFDSQINVPKTMESGTFITDKTVLDMKAMDSKSNGAIAWSNQTSF | |
| S112T | TCQDIFKETNATYPSSDVPCDATLTEKSFETDMNLNFQNLSVMGLRILLLK | |
| TRBV26*01 | VAGFNLLMTLRLWSSGSGATNFSLLKQAGDVEENPGPMATRLLCYTVLCL | |
| with regular | LGARILNSKVIQTPRYLVKGQGQKAKMRCIPEKGHPVVFWYQQNKNNEF | |
| murine | KFLINFQNQEVLQQIDMTEKRFSAECPSNSPCSLEIQSSEAGDSALYLCASS | |
| constant | LGLGDYEQYFGPGTRLTVLEDLRNVTPPKVSLFEPSKAEIANKQKATLVCLA | |
| region | RGFFPDHVELSWWVNGKEVHSGVSTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS (SEQ ID | |
| | NO: 41) | |
| CT 538: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 119) |
| TCR TRAV8- | DTAVYFCATDFTSGGNYKFGKGTSLVVHPDIQNPEPAVYQLKDPRSQDST | |
| 1*01 L110F | LCLFTDFDSQINVPKTMESGTFITDKTVLDMKAMDSKSNGAIAWSNQTSF | |
| TRBV26*01 | TCQDIFKETNATYPSSDVPCDATLTEKSFETDMNLNFQNLSVMGLRILLLK | |
| L117T with | VAGFNLLMTLRLWSSGSGATNFSLLKQAGDVEENPGPMATRLLCYTVLCL | |
| regular | LGARILNSKVIQTPRYLVKGQGQKAKMRCIPEKGHPVVFWYQQNKNNEF | |
| murine | KFLINFQNQEVLQQIDMTEKRFSAECPSNSPCSLEIQSSEAGDSALYLCASS | |
| constant | LGTGDYEQYFGPGTRLTVLEDLRNVTPPKVSLFEPSKAEIANKQKATLVCLA | |
| region | RGFFPDHVELSWWVNGKEVHSGVSTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS (SEQ ID | |
| | NO: 42) | |
| CT 539: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 120) |
| TCR TRAV8- | DTAVYFCATDLTTGGNYKFGKGTSLVVHPDIQNPEPAVYQLKDPRSQDST | |
| 1*01 S112T | LCLFTDFDSQINVPKTMESGTFITDKTVLDMKAMDSKSNGAIAWSNQTSF | |
| TRBV26*01 | TCQDIFKETNATYPSSDVPCDATLTEKSFETDMNLNFQNLSVMGLRILLLK | |
| L117T with | VAGFNLLMTLRLWSSGSGATNFSLLKQAGDVEENPGPMATRLLCYTVLCL | |
| regular | LGARILNSKVIQTPRYLVKGQGQKAKMRCIPEKGHPVVFWYQQNKNNEF | |
| murine | KFLINFQNQEVLQQIDMTEKRFSAECPSNSPCSLEIQSSEAGDSALYLCASS | |
| constant | LGTGDYEQYFGPGTRLTVLEDLRNVTPPKVSLFEPSKAEIANKQKATLVCLA | |
| region | RGFFPDHVELSWWVNGKEVHSGVSTDPQAYKESNYSYCLSSRLRVSATF | |
| | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS (SEQ ID | |
| | NO: 43) | |
| CT 540: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 121) |
| TCR TRAV8- | DTAVYFCATDFTTGGNYKFGKGTSLVVHPDIQNPEPAVYQLKDPRSQDST | |
| 1*01 L110F & | LCLFTDFDSQINVPKTMESGTFITDKTVLDMKAMDSKSNGAIAWSNQTSF | |
| S112T | TCQDIFKETNATYPSSDVPCDATLTEKSFETDMNLNFQNLSVMGLRILLLK | |
| TRBV26*01 | VAGFNLLMTLRLWSSGSGATNFSLLKQAGDVEENPGPMATRLLCYTVLCL | |
| L117T with | LGARILNSKVIQTPRYLVKGQGQKAKMRCIPEKGHPVVFWYQQNKNNEF | |
| regular | KFLINFQNQEVLQQIDMTEKRFSAECPSNSPCSLEIQSSEAGDSALYLCASS | |
| murine | LGTGDYEQYFGPGTRLTVLEDLRNVTPPKVSLFEPSKAEIANKQKATLVCLA | |
| constant | RGFFPDHVELSWWVNGKEVHSGVSTDPQAYKESNYSYCLSSRLRVSATF | |
| region | WHNPRNHFRCQVQFHGLSEEDKWPEGSPKPVTQNISAEAWGRADCGIT | |
| | SASYQQGVLSATILYEILLGKATLYAVLVSTLVVMAMVKRKNS | |
| | (SEQ ID NO: 44) | |
| CT 541: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 122) |
| TCR TRAV8- | DTAVYFCATDFTSGGNYKFGKGTSLVVHPNIQNPEPAVYQLKDPRSQDST | |
| 1*01 L110F | LCLFTDFDSQINVPKTMESGTFITDKCVLDMKAMDSKSNGAIAWSNQTSF | |
| TRBV26*01 | TCQDIFKETNATYPSSDVPCDATLTEKSFETDMNLNFQNLLVIVLRILLLKVA | |
| with murine | GFNLLMTLRLWSSGSGATNFSLLKQAGDVEENPGPMATRLLCYTVLCLLG | |
| constant | ARILNSKVIQTPRYLVKGQGQKAKMRCIPEKGHPVVFWYQQNKNNEFKF | |
| region | LINFONQEVLQQIDMTEKRFSAECPSNSPCSLEIQSSEAGDSALYLCASSLG | |
| | LGDYEQYFGPGTRLTVLEDLRNVTPPKVSLFEPSKAEIANKQKATLVCLARG | |
| | FFPDHVELSWWVNGKEVHSGVCTDPQAYKESNYSYCLSSRLRVSATFWH | |
| | NPRNHFRCQVQFHGLSEEDKWPEGSPKPVTQNISAEAWGRADCGITSAS | |
| | YQQGVLSATILYEILLGKATLYAVLVSTLVVMAMVKRKNS (SEQ ID NO: | |
| | 45) | |
| CT 542: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 123) |
| TCR TRAV8- | DTAVYFCATDLTTGGNYKFGKGTSLVVHPNIQNPEPAVYQLKDPRSQDST | |
| 1*01 S112T | LCLFTDFDSQINVPKTMESGTFITDKCVLDMKAMDSKSNGAIAWSNQTSF | |
| TRBV26*01 | TCQDIFKETNATYPSSDVPCDATLTEKSFETDMNLNFQNLLVIVLRILLLKVA | |
| with murine | GFNLLMTLRLWSSGSGATNFSLLKQAGDVEENPGPMATRLLCYTVLCLLG | |
| constant | ARILNSKVIQTPRYLVKGQGQKAKMRCIPEKGHPVVFWYQQNKNNEFKF | |
| region | LINFQNQEVLQQIDMTEKRFSAECPSNSPCSLEIQSSEAGDSALYLCASSLG | |
| | LGDYEQYFGPGTRLTVLEDLRNVTPPKVSLFEPSKAEIANKQKATLVCLARG | |
| | FFPDHVELSWWVNGKEVHSGVCTDPQAYKESNYSYCLSSRLRVSATFWH | |
| | NPRNHFRCQVQFHGLSEEDKWPEGSPKPVTQNISAEAWGRADCGITSAS | |
| | YQQGVLSATILYEILLGKATLYAVLVSTLVVMAMVKRKNS | |
| | (ESQ ID NO: 46) | |
| CT 543: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 124) |
| TCR TRAV8- | DTAVYFCATDFTTGGNYKFGKGTSLVVHPNIQNPEPAVYQLKDPRSQDST | |
| 1*01 L110F & | LCLFTDFDSQINVPKTMESGTFITDKCVLDMKAMDSKSNGAIAWSNQTSF | |
| S112T | TCQDIFKETNATYPSSDVPCDATLTEKSFETDMNLNFQNLLVIVLRILLLKVA | |
| TRBV26*01 | GFNLLMTLRLWSSGSGATNFSLLKQAGDVEENPGPMATRLLCYTVLCLLG | |
| with murine | ARILNSKVIQTPRYLVKGQGQKAKMRCIPEKGHPVVFWYQQNKNNEFKF | |
| constant | LINFQNQEVLQQIDMTEKRFSAECPSNSPCSLEIQSSEAGDSALYLCASSLG | |
| region | LGDYEQYFGPGTRLTVLEDLRNVTPPKVSLFEPSKAEIANKQKATLVCLARG | |
| | FFPDHVELSWWVNGKEVHSGVCTDPQAYKESNYSYCLSSRLRVSATFWH | |
| | NPRNHFRCQVQFHGLSEEDKWPEGSPKPVTQNISAEAWGRADCGITSAS | |
| | YQQGVLSATILYEILLGKATLYAVLVSTLVVMAMVKRKNS | |
| | (SEQ ID NO: 47) | |
| CT 544: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 125) |
| TCR TRAV8- | DTAVYFCATDLTSGGNYKFGKGTSLVVHPNIQNPEPAVYQLKDPRSQDST | |
| 1*01 | LCLFTDFDSQINVPKTMESGTFITDKCVLDMKAMDSKSNGAIAWSNQTSF | |
| TRBV26*01 | TCQDIFKETNATYPSSDVPCDATLTEKSFETDMNLNFQNLLVIVLRILLLKVA | |
| L117T with | GFNLLMTLRLWSSGSGATNFSLLKQAGDVEENPGPMATRLLCYTVLCLLG | |
| murine | ARILNSKVIQTPRYLVKGQGQKAKMRCIPEKGHPVVFWYQQNKNNEFKF | |
| constant | LINFQNQEVLQQIDMTEKRFSAECPSNSPCSLEIQSSEAGDSALYLCASSLG | |
| region | TGDYEQYFGPGTRLTVLEDLRNVTPPKVSLFEPSKAEIANKQKATLVCLAR | |
| | GFFPDHVELSWWVNGKEVHSGVCTDPQAYKESNYSYCLSSRLRVSATFW | |
| | HNPRNHFRCQVQFHGLSEEDKWPEGSPKPVTQNISAEAWGRADCGITSA | |
| | SYQQGVLSATILYEILLGKATLYAVLVSTLVVMAMVKRKNS | |
| | (SEQ ID NO: 48) | |
| CT 545: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 126) |
| TCR TRAV8- | DTAVYFCATDFTSGGNYKFGKGTSLVVHPNIQNPEPAVYQLKDPRSQDST | |
| 1*01 L110F | LCLFTDFDSQINVPKTMESGTFITDKCVLDMKAMDSKSNGAIAWSNQTSF | |
| TRBV26*01 | TCQDIFKETNATYPSSDVPCDATLTEKSFETDMNLNFQNLLVIVLRILLLKVA | |
| L117T with | GFNLLMTLRLWSSGSGATNFSLLKQAGDVEENPGPMATRLLCYTVLCLLG | |
| murine | ARILNSKVIQTPRYLVKGQGQKAKMRCIPEKGHPVVFWYQQNKNNEFKF | |
| constant | LINFQNQEVLQQIDMTEKRFSAECPSNSPCSLEIQSSEAGDSALYLCASSLG | |
| region | TGDYEQYFGPGTRLTVLEDLRNVTPPKVSLFEPSKAEIANKQKATLVCLAR | |
| | GFFPDHVELSWWVNGKEVHSGVCTDPQAYKESNYSYCLSSRLRVSATFW | |
| | HNPRNHFRCQVQFHGLSEEDKWPEGSPKPVTQNISAEAWGRADCGITSA | |
| | SYQQGVLSATILYEILLGKATLYAVLVSTLVVMAMVKRKNS (SEQ ID NO: | |
| | 49) | |
| CT 546: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 127) |
| TCR TRAV8- | DTAVYFCATDLTTGGNYKFGKGTSLVVHPNIQNPEPAVYQLKDPRSQDST | |
| 1*01 S112T | LCLFTDFDSQINVPKTMESGTFITDKCVLDMKAMDSKSNGAIAWSNQTSF | |
| TRBV26*01 | TCQDIFKETNATYPSSDVPCDATLTEKSFETDMNLNFQNLLVIVLRILLLKVA | |
| L117T with | GFNLLMTLRLWSSGSGATNFSLLKQAGDVEENPGPMATRLLCYTVLCLLG | |
| murine | ARILNSKVIQTPRYLVKGQGQKAKMRCIPEKGHPVVFWYQQNKNNEFKF | |
| constant | LINFQNQEVLQQIDMTEKRFSAECPSNSPCSLEIQSSEAGDSALYLCASSLG | |
| region | TGDYEQYFGPGTRLTVLEDLRNVTPPKVSLFEPSKAEIANKQKATLVCLAR | |
| | GFFPDHVELSWWVNGKEVHSGVCTDPQAYKESNYSYCLSSRLRVSATFW | |
| | HNPRNHFRCQVQFHGLSEEDKWPEGSPKPVTQNISAEAWGRADCGITSA | |
| | SYQQGVLSATILYEILLGKATLYAVLVSTLVVMAMVKRKNS (SEQ ID NO: | |
| | 50) | |
| CT 547: | MHSLLGLLMVSLWLQLTRVNSQLAEENPWALSVHEGESVTVNCSYKTSIT | (SEQ ID NO: |
| pLenti 1 CEA | ALQWYRQKSGEGPAQLILIRSNEREKRNGRLRATLDTSSQSSSLSITATRCE | 128) |
| TCR TRAV8- | DTAVYFCATDFTTGGNYKFGKGTSLVVHPNIQNPEPAVYQLKDPRSQDST | |
| 1*01 L110F & | LCLFTDFDSQINVPKTMESGTFITDKCVLDMKAMDSKSNGAIAWSNQTSF | |
| S112T | TCQDIFKETNATYPSSDVPCDATLTEKSFETDMNLNFQNLLVIVLRILLLKVA | |
| TRBV26*01 | GFNLLMTLRLWSSGSGATNFSLLKQAGDVEENPGPMATRLLCYTVLCLLG | |
| L117T with | ARILNSKVIQTPRYLVKGQGQKAKMRCIPEKGHPVVFWYQQNKNNEFKF | |
| murine | LINFQNQEVLQQIDMTEKRFSAECPSNSPCSLEIQSSEAGDSALYLCASSLG | |
| constant | TGDYEQYFGPGTRLTVLEDLRNVTPPKVSLFEPSKAEIANKQKATLVCLAR | |
| region | GFFPDHVELSWWVNGKEVHSGVCTDPQAYKESNYSYCLSSRLRVSATFW | |
| | HNPRNHFRCQVQFHGLSEEDKWPEGSPKPVTQNISAEAWGRADCGITSA | |
| | SYQQGVLSATILYEILLGKATLYAVLVSTLVVMAMVKRKNS (SEQ ID NO: | |
| | 51) | |

In some embodiments, the first receptor comprises a sequence at least 80% identical, at least 85% identical, at least 90% identical, at least 95% identical, at least 96% identical, at least 97% identical, at least 98% identical, at least 99% identical, or at least 99.5% identical to a sequence or subsequence of any one of SEQ ID NOS: 16-31 or 36-51. In some embodiments, the first receptor comprises a sequence or subsequence of any one of SEQ ID NOS: 16-31 or 36-51.

In some embodiments, the first receptor comprises a TCR alpha chain comprising or consisting essentially of amino acids 1-270 of any one of SEQ ID NOS: 16-31, or a sequence that is at least 80% identical, at least 85% identical, at least 90% identical, at least 95% identical, at least 96% identical, at least 97% identical, at least 98% identical, at least 99% identical, or at least 99.5% identical thereto. In some embodiments, the first receptor comprises a TCR alpha chain comprising or consisting essentially of amino acids 1-270 of any one of SEQ ID NOS: 16-31.

In some embodiments, the first receptor comprises a TCR beta chain comprising or consisting essentially of amino acids 293-598 of any one of SEQ ID NOS: 16-31, or a sequence that is at least 80% identical, at least 85% identical, at least 90% identical, at least 95% identical, at least 96% identical, at least 97% identical, at least 98% identical, at least 99% identical, or at least 99.5% identical thereto. In some embodiments, the first receptor comprises a TCR beta chain comprising or consisting essentially of amino acids 293-598 of any one of SEQ ID NOS: 16-31.

In some embodiments, the first receptor comprises a TCR alpha chain comprising amino acids 1-270 of any one of SEQ ID NOS: 16-31, and a TCR beta chain comprising amino acids 293-598 of any one of SEQ ID NOS: 16-31.

In some embodiments, the first receptor comprises a TCR alpha chain comprising or consisting essentially of amino acids 1-268 of any one of SEQ ID NOS: 36-51, or a sequence that is at least 80% identical, at least 85% identical, at least 90% identical, at least 95% identical, at least 96% identical, at least 97% identical, at least 98% identical, at least 99% identical, or at least 99.5% identical thereto. In some embodiments, the first receptor comprises a TCR alpha chain comprising or consisting essentially of amino acids 1-268 of any one of SEQ ID NOS: 36-51.

In some embodiments, the first receptor comprises a TCR beta chain comprising or consisting essentially of amino acids 291-596 of any one of SEQ ID NOS: 36-51, or a sequence that is at least 80% identical, at least 85% identical, at least 90% identical, at least 95% identical, at least 96% identical, at least 97% identical, at least 98% identical, at least 99% identical, or at least 99.5% identical thereto. In some embodiments, the first receptor comprises a TCR beta chain comprising or consisting essentially of amino acids 291-596 of any one of SEQ ID NOS: 36-51.

In some embodiments, the first receptor comprises a TCR alpha chain comprising amino acids 1-268 of any one of SEQ ID NOS: 36-51, and a TCR beta chain comprising amino acids 291-596 of any one of SEQ ID NOS: 36-51.

In some embodiments, the extracellular ligand binding domain of the first receptor is an ScFv. In some embodiments, the ScFv domain binds to CEA. In some embodiments, the ScFv is the ligand binding domain of a CAR. Exemplary CAR sequences comprising CEA targeting ScFv domains are shown in Table 3 below. In Table 3, CDR sequences are underlined.

TABLE 3
Exemplary CARs with ScFv that target CEA

| | | Nucleotide |
|---|---|---|
| Name | Protein Sequence | Sequence |
| CT | MDMRVPAQLLGLLLLWLRGARCQVQLVQSGSELKKPGASVKVSCK | (SEQ ID NO: 129) |
| 618 | ASGYTFTEFGMNWVRQAPGQGLEWMGWINTKTGEATYVEEFKG | |
| | RFVFSLDTSVSTAYLQISSLKAEDTAVYYCARWDFAYYVEAMDYWG | |
| | QGTTVTVSSGGGGSGGGGSGGGGSGGDIQMTQSPSSLSASVGDR | |
| | VTITCKASQNVGTNVAWYQQKPGKAPKLLIYSASYRYSGVPSRFSGS | |
| | GSGTDFTLTISSLQPEDFATYYCHQYYTYPLFTFGQGTKLEIKTTTPAP | |
| | RPPTPAPTIASQPLSLRPEACRPAAGGAVHTRGLDFACDFWVLVVV | |
| | GGVLACYSLLVTVAFIIFWVRSKRSRLLHSDYMNMTPRRPGPTRKHY | |
| | QPYAPPRDFAAYRSKRGRKKLLYIFKQPFMRPVQTTQEEDGCSCRFP | |
| | EEEEGGCELRVKFSRSADAPAYKQGQNQLYNELNLGRREEYDVLDK | |
| | RRGRDPEMGGKPRRKNPQEGLYNELQKDKMAEAYSEIGMKGERR | |
| | RGKGHDGLYQGLSTATKDTYDALHMQALPPR (SEQ ID NO: 52) | |
| CT | MDMRVPAQLLGLLLLWLRGARCQVQLVQSGAEVKKPGASVKVSCK | (SEQ ID NO: 130) |
| 619 | ASGYTFTEFGMNWVRQAPGQGLEWMGWINTKTGEATYVEEFKG | |
| | RVTFTTDTSTSTAYMELRSLRSDDTAVYYCARWDFAYYVEAMDYW | |
| | GQGTTVTVSSGGGGSGGGGSGGGGSGGDIQMTQSPSSLSASVGD | |
| | RVTITCKASAAVGTYVAWYQQKPGKAPKLLIYSASYRKRGVPSRFSG | |
| | SGSGTDFTLTISSLQPEDFATYYCHQYYTYPLFTFGQGTKLEIKRTTTT | |
| | PAPRPPTPAPTIASQPLSLRPEACRPAAGGAVHTRGLDFACDFWVL | |
| | VVVGGVLACYSLLVTVAFIIFWVRSKRSRLLHSDYMNMTPRRPGPTR | |
| | KHYQPYAPPRDFAAYRSKRGRKKLLYIFKQPFMRPVQTTQEEDGCS | |
| | CRFPEEEEGGCELRVKFSRSADAPAYKQGQNQLYNELNLGRREEYD | |
| | VLDKRRGRDPEMGGKPRRKNPQEGLYNELQKDKMAEAYSEIGMK | |
| | GERRRGKGHDGLYQGLSTATKDTYDALHMQALPPR (SEQ ID NO: | |
| | 53) | |
| CT | MDMRVPAQLLGLLLLWLRGARCQVQLVQSGSELKKPGASVKVSCK | (SEQ ID NO: 131) |
| 620 | ASGYTFTEFGMNWVRQAPGQGLEWMGWINTKTGEATYVEEFKG | |
| | RFVFSLDTSVSTAYLQISSLKAEDTAVYYCARWDFAHYFQTMDYWG | |
| | QGTTVTVSSGGGGSGGGGSGGGGSGGDIQMTQSPSSLSASVGDRVT | |
| | ITCKASAAVGTYVAWYQQKPGKAPKLLIYSASYRKRGVPSRFSGSGS | |
| | GTDFTLTISSLQPEDFATYYCHQYYTYPLFTFGQGTKLEIKRTTTPA | |
| | PRPPTPAPTIASQPLSLRPEACRPAAGGAVHTRGLDFACDFWVLVV | |
| | VGGVLACYSLLVTVAFIIFWVRSKRSRLLHSDYMNMTPRRPGPTRKH | |
| | YQPYAPPRDFAAYRSKRGRKKLLYIFKQPFMRPVQTTQEEDGCSCRF | |
| | PEEEEGGCELRVKFSRSADAPAYKQGQNQLYNELNLGRREEYDVLD | |
| | KRRGRDPEMGGKPRRKNPQEGLYNELQKDKMAEAYSEIGMKGER | |
| | RRGKGHDGLYQGLSTATKDTYDALHMQALPPR (SEQ ID NO: 54) | |

In some embodiments, a CEA ScFv comprises a CDR-H1 of EFGMN (SEQ ID NO: 55), a CDR-H2 of WINTKTGEATYVEEFKG (SEQ ID NO: 56), a CDR-H3 of WDFAYYVEAMDY (SEQ ID NO: 57) or WDFAHYFQTMDY (SEQ ID NO: 58), a CDR-L1 of KASQNVGTNVA (SEQ ID NO: 59) or KASAAVGTYVA (SEQ ID NO: 60), a CDR-L2 of SASYRYS (SEQ ID NO: 61) or SASYRKR, and a CDR-L3 of HQYYTYPLFT (SEQ ID NO: 63) or sequences having at least 85% or at least 95% identity thereto. In some embodiments, a CEA ScFv comprises a CDR-H1 of EFGMN (SEQ ID NO: 55), a CDR-H2 of WINTKTGEATYVEEFKG (SEQ ID NO: 56), a CDR-H3 of WDFAYYVEAMDY (SEQ ID NO: 57) or WDFAHYFQTMDY (SEQ ID NO: 58), a CDR-L1 of KASQNVGTNVA (SEQ ID NO: 59) or KASAAVGTYVA (SEQ ID NO: 60), a CDR-L2 of SASYRYS (SEQ ID NO: 61) or SASYRKR (SEQ ID NO: 62) and a CDR-L3 of HQYYTYPLFT (SEQ ID NO: 63). In some embodiments, a CEA ScFv comprises a CDR-H1 of EFGMN (SEQ ID NO: 55), a CDR-H2 of WINTKTGEATYVEEFKG (SEQ ID NO: 56), a CDR-H3 of WDFAYYVEAMDY (SEQ ID NO: 57), a CDR-L1 of KASQNVGTNVA (SEQ ID NO: 59), a CDR-L2 of SASYRYS (SEQ ID NO: 61) and a CDR-L3 of HQYYTYPLFT (SEQ ID NO: 63). In some embodiments, a CEA ScFv comprises a CDR-H1 of EFGMN (SEQ ID NO: 55), a CDR-H2 of WINTKTGEATYVEEFKG (SEQ ID NO: 56), a CDR-H3 of WDFAYYVEAMDY (SEQ ID NO: 57), a CDR-L1 of KASAAVGTYVA (SEQ ID NO: 60), a CDR-L2 of SASYRKR (SEQ ID NO: 62), and a CDR-L3 of HQYYTYPLFT (SEQ ID NO: 63). In some embodiments, a CEA ScFv comprises a CDR-H1 of EFGMN (SEQ ID NO: 56), a CDR-H2 of WINTKTGEATYVEEFKG (SEQ ID NO: 56), a CDR-H3 of WDFAHYFQTMDY (SEQ ID NO: 58), a CDR-L1 of KASAAVGTYVA (SEQ ID NO: 60), a CDR-L2 of SASYRKR (SEQ ID NO: 62), and a CDR-L3 of HQYYTYPLFT (SEQ ID NO: 63).

Exemplary ScFv that recognize CEA are shown in Table 4 below. Underlining indicates CDR sequences.

TABLE 4
Exemplary ScFv that target CEA

| Protein sequence | DNA sequence |
|---|---|
| MFE23M: | (SEQ ID NO: 132) |
| QVQLQQSGAELVRSGTSVKLSCTASGFNIK | |
| DSYMHWLRQGPEQGLEWIGWIDPENGDTEY | |
| APKFQGKATFTTDTSSNTAYLQLSSLTSED | |
| TAVYYCNEGTPTGPYYFDYWGQGTTVTVSS | |
| GGGGSGGGGSGGGGSGGENVLTQSPAIMSA | |
| SPGEKVTITCSASSSVSYMHWFQQKPGTSP | |
| KLWIYSTSNLASGVPARFSGSGSGTSYSLT | |
| ISRMEAEDAATYYCQQRSSYPLTFGAGTKL | |
| ELK | |
| (SEQ ID NO: 64) | |
| MFE23H: | (SEQ ID NO: 133) |
| QVQLVQSGAEVKKPGASVKVSCKASGFNIK | |
| DSYMHWVRQAPGQGLEWMGWIDPENGDTEY | |
| APKFQGRVTMTTDTSTSTAYMELRSLRSDD | |
| TAVYYCNEGTPTGPYYFDYWGQGTTVTVSS | |
| GGGGSGGGGSGGGGSGGEIVLTQSPATLSL | |
| SPGERATLSCSASSSVSYMHWYQQKPGLAP | |
| RLLIYSTSNLASGIPDRFSGSGSGTDFTLT | |
| ISRLEPEDFAVYYCQQRSSYPLTFGQGTKL | |
| EIK | |
| (SEQ ID NO: 65) | |
| E8: | (SEQ ID NO: 134) |
| EVQLAESGGGLVQPGGSLRLSCAASGFTFS | |
| SDAMSWVRQAPGKGLEWVSAISGSGGSTYY | |
| ADSVKGRFTISRDNSKNTLYLQMNSLRAED | |
| TAVYYCAKSNEFLFDYWGQGTLVTVSSGGG | |
| GSGGGGSGGGGSGGSSELTQDPAVSVALGQ | |
| TVRITCQGDSLRSSYASWYRQRPGQAPVLV | |
| IYGKNNRPSGIPDRFSGSSSGNTASLTITG | |
| AQAEDEADYYWNSSYAWLPYVVFGGGTKLT | |
| VLG | |
| (SEQ ID NO: 66) | |
| SM3E: | (SEQ ID NO: 135) |
| QVQLEQSGAGVVKPGASVKLSCKASGFNIK | |
| DSYMHWLRQGPGQRLEWIGWIDPENGDTEY | |
| APKFQGKATFTTDTSANTAYLGLSSLRPED | |
| TAVYYCNEGTPTGPYYFDYWGQGTLVTVSS | |
| GGGGSGGGGSGGGGSGGENVLTQSPSSMSV | |
| SVGDRVNIACSASSSVPYMHWLQQKPGKSP | |
| KLLIYLTSNLASGVPSRFSGSGSGTDYSLT | |
| ISSVQPEDAATYYCQQRSSYPLTFGGGTKL | |
| EIK | |
| (SEQ ID NO: 67) | |
| CT618: | (SEQ ID NO: 136) |
| QVQLVQSGSELKKPGASVKVSCKASGYTFT | |
| EFGMNWVRQAPGQGLEWMGWINTKTGEATY | |
| VEEFKGRFVFSLDTSVSTAYLQISSLKAED | |
| TAVYYCARWDFAYYVEAMDYWGQGTTVTVS | |
| SGGGGSGGGGSGGGGSGGDIQMTQSPSSLS | |
| ASVGDRVTITCKASQNVGTNVAWYQQKPGK | |
| APKLLIYSASYRYSGVPSRFSGSGSGTDFT | |
| LTISSLQPEDFATYYCHQYYTYPLFTFGQG | |
| TKLEIK | |
| (SEQ ID NO: 68) | |
| CT619: | (SEQ ID NO: 137) |
| QVQLVQSGAEVKKPGASVKVSCKASGYTFT | |
| EFGMNWVRQAPGQGLEWMGWINTKTGEATY | |
| VEEFKGRVTFTTDTSTSTAYMELRSLRSDD | |
| TAVYYCARWDFAYYVEAMDYWGQGTTVTVS | |
| SGGGGSGGGGSGGGGSGGDIQMTQSPSSLS | |
| ASVGDRVTITCKASAAVGTYVAWYQQKPGK | |
| APKLLIYSASYRKRGVPSRFSGSGSGTDFT | |
| LTISSLOPEDFATYYCHQYYTYPLFTFGQG | |
| TKLEIK | |
| (SEQ ID NO: 69) | |
| CT620: | (SEQ ID NO: 138) |
| QVQLVQSGSELKKPGASVKVSCKASGYTFT | |
| EFGMNWVRQAPGQGLEWMGWINTKTGEATY | |
| VEEFKGRFVFSLDTSVSTAYLQISSLKAED | |
| TAVYYCARWDFAHYFQTMDYWGQGTTVTVS | |
| SGGGGSGGGGSGGGGSGGDIQMTQSPSSLS | |
| ASVGDRVTITCKASAAVGTYVAWYQQKPGK | |
| APKLLIYSASYRKRGVPSRFSGSGSGTDFT | |
| LTISSLQPEDFATYYCHQYYTYPLFTFGQG | |
| TKLEIK | |
| (SEQ ID NO: 70) | |

In some embodiments, a CEA ScFv comprises a sequence selected from the group consisting of SEQ ID NOs: 64-70, or a sequence having at least 85%, at least 90%, at least 95%, at least 97% or at least 99% identity thereto. In some embodiments, a CEA ScFv comprises, or consists essentially of, a sequence selected from the group consisting of SEQ ID NOs: 64-70.

### Chimeric Antigen Receptors (CARs)

The disclosure provides a first, activator receptor and immune cells comprising same. In some embodiments, the first receptor is a chimeric antigen receptor.

The term “chimeric antigen receptors (CARs)” as used herein, may refer to artificial receptors derived from T-cell receptors and encompasses engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen, for example. Exemplary CARs comprise an intracellular activation domain, a transmembrane domain, and an extracellular domain comprising a tumor associated antigen binding region. In some embodiments, CARs further comprise a hinge domain. In particular aspects, CARs comprise fusions of single-chain variable fragments (scFv) derived from monoclonal antibodies, fused to a CD3 transmembrane domain and endodomain. The specificity of other CAR designs may be derived from ligands of receptors (e.g., peptides). In certain cases, CARs comprise domains for additional co-stimulatory signaling, such as CD3, 4-1BB, FcR, CD27, CD28, CD137, DAP10, and/or OX40. In some cases, molecules can be co-expressed with the CAR, including co-stimulatory molecules, reporter genes for imaging, gene products that conditionally ablate the T cells upon addition of a pro-drug, homing receptors, cytokines, and cytokine receptors.

In some embodiments, the extracellular ligand binding domain of the first receptor is fused to the extracellular domain of a CAR.

In some embodiments, the CARs of the present disclosure comprise an extracellular hinge region. Incorporation of a hinge region can affect cytokine production from CAR-T cells and improve expansion of CAR-T cells in vivo. Exemplary hinges can be isolated or derived from IgD and CD8 domains, for example IgG1. In some embodiments, the hinge is isolated or derived from CD8a or CD28.

In some embodiments, the hinge is isolated or derived from CD8a or CD28. In some embodiments, the CD8a hinge comprises an amino acid sequence having at least 80% identity, at least 90% identity, at least 95% identity, at least 99% identity or is identical to a sequence of TTTPAPRPPTPAPTIASQPLSLRPEACRPAAGGAVHTRGLDFACD (SEQ ID NO: 71). In some embodiments, the CD8a hinge comprises SEQ ID NO: 71. In some embodiments, the CD8a hinge consists essentially of SEQ ID NO: 71. In some embodiments, the CD8a hinge is encoded by a nucleotide sequence having at least 80% identity, at least 90% identity, at least 95% identity, at least 99% identity or is identical to a sequence of

(SEQ ID NO: 72)
ACCACGACGCCAGCGCCGCGACCACCAACACCGGCGCCCACCATCGC
GTCGCAGCCCCTGTCCCTGCGCCCAGAGGCGTGCCGGCCAGCGGCGG
GGGGCGCAGTGCACACGAGGGGGCTGGACTTCGCCTGTGAT.

In some embodiments, the CD8a hinge is encoded by SEQ ID NO: 72.

In some embodiments, the CD28 hinge comprises an amino acid sequence having at least 80% identity, at least 90% identity, at least 95% identity, at least 99% identity or is identical to a sequence of CTIEVMYPPPYLDNEKSNGTIIHVKGKHLCPSPLFPGPSKP (SEQ ID NO: 73). In some embodiments, the CD28 hinge comprises or consists essentially of SEQ ID NO: 73. In some embodiments, the CD28 hinge is encoded by a nucleotide sequence having at least 80% identity, at least 90% identity, at least 95% identity, at least 99% identity or is identical to a sequence of TGTACCATTGAAGTTATGTATCCTCCTCCTTACCTAGACAATGAGAAGAGCAATGGAAC CATTATCCATGTGAAAGGGAAACACCTTTGTCCAAGTCCCCTATTTCCCGGACCTTCTA AGCCC (SEQ ID NO: 74). In some embodiments, the CD28 hinge is encoded by SEQ ID NO: 74.

The CARs of the present disclosure can be designed to comprise a transmembrane domain that is fused to the extracellular domain of the CAR. In some embodiments, the transmembrane domain that naturally is associated with one of the domains in the CAR is used. For example, a CAR comprising a CD28 co-stimulatory domain might also use a CD28 transmembrane domain. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions may be isolated or derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, or from an immunoglobulin such as IgG4. Alternatively the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

In some embodiments of the CARs of the disclosure, the CARs comprise a CD28 transmembrane domain. In some embodiments, the CD28 transmembrane domain comprises an amino acid sequence having at least 80% identity, at least 90% identity, at least 95% identity, at least 99% identity or is identical to a sequence of FWVLVVVGGVLACYSLLVTVAFIIFWV (SEQ ID NO: 75). In some embodiments, the CD28 transmembrane domain comprises or consists essentially of SEQ ID NO: 75. In some embodiments, the CD28 transmembrane domain is encoded by a nucleotide sequence having at least 80% identity, at least 90% identity, at least 95% identity, at least 99% identity or is identical to a sequence of

(SEQ ID NO: 76)
TTCTGGGTGCTGGTCGTTGTGGGCGGCGTGCTGGCCTGCT
ACAGCCTGCTGGTGACAGTGGCCTTCATCATCTTTTGGGTG.
In some embodiments, the CD28 transmembrane
domain is encoded by SEQ ID NO: 76.

In some embodiments of the CARs of the disclosure, the CARs comprise an IL-2Rbeta transmembrane domain. In some embodiments, the IL-2Rbeta transmembrane domain comprises an amino acid sequence having at least 80% identity, at least 90% identity, at least 95% identity, at least 99% identity or is identical to a sequence of IPWLGHLLVGLSGAFGFIILVYLLI (SEQ ID NO: 77). In some embodiments, the IL-2Rbeta transmembrane domain comprises or consists essentially of SEQ ID NO: 77. In some embodiments, the IL-2Rbeta transmembrane domain is encoded by a nucleotide sequence having at least 80% identity, at least 90% identity, at least 95% identity, at least 99% identity or is identical to a sequence of

ATTCCGTGGC TCGGCCACCT CCTCGTGGGC CTCAGCGGGG CTTTTGGCTT CATCATCTTA GTGTACTTGC TGATC (SEQ ID NO: 78). In some embodiments, the IL-2Rbeta transmembrane domain is encoded by SEQ ID NO: 78.

The cytoplasmic domain or otherwise the intracellular signaling domain of the CARs of the instant invention is responsible for activation of at least one of the normal effector functions of the immune cell in which the CAR has been placed. The term “effector function” refers to a specialized function of a cell. Thus the term “intracellular signaling domain” refers to the portion of a protein which transduces the effector function signal and directs the cell to perform a specialized function. While usually the entire intracellular signaling domain can be employed, in many cases it is not necessary to use the entire domain. To the extent that a truncated portion of the intracellular signaling domain is used, such truncated portion may be used in place of the intact chain as long as it transduces the effector function signal. In some cases, multiple intracellular domains can be combined to achieve the desired functions of the CAR-T cells of the instant disclosure. The term intracellular signaling domain is thus meant to include any truncated portion of one or more intracellular signaling domains sufficient to transduce the effector function signal.

Examples of intracellular signaling domains for use in the CARs of the instant disclosure include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

Accordingly, the intracellular domain of CARs of the instant disclosure comprises at least one cytoplasmic activation domain. In some embodiments, the intracellular activation domain ensures that there is T-cell receptor (TCR) signaling necessary to activate the effector functions of the CAR T-cell. In some embodiments, the at least one cytoplasmic activation is a CD247 molecule (CD3ζ) activation domain, a stimulatory killer immunoglobulin-like receptor (KIR) KIR2DS2 activation domain, or a DNAX-activating protein of 12 kDa (DAP12) activation domain.

In some embodiments, the CD3ζ activation domain comprises an amino acid sequence having at least 80% identity, at least 90% identity, at least 95% identity, at least 99% identity or is identical to a sequence of

(SEQ ID NO: 79)
RVKFSRSADAPAYKQGQNQLYNELNLGRREEYDVLDKRR
GRDPEMGGKPRRKNPQEGLYNELQKDKMAEAYSEIGMKG
ERRRGKGHDGLYQGLSTATKDTYDALHMQALPPR.

In some embodiments, the CD3ζ activation domain comprises or consists essentially of SEQ ID NO: 79. In some embodiments, the CD3ζ activation domain is encoded by a nucleotide sequence having at least 80% identity, at least 90% identity, at least 95% identity, at least 99% identity or is identical to a sequence of

AGAGTGAAGTTCAGCAGGAGCGCAGACGCCCCCGCGTACAAGCAGGGCCAGAACCAGCTCTATAACGAGCT CAATCTAGGACGAAGAGAGGAGTACGATGTTTTGGACAAGCGTAGAGGCCGGGACCCTGAGATGGGGGGAA AGCCGAGAAGGAAGAACCCTCAGGAAGGCCTGTACAATGAACTGCAGAAAGATAAGATGGCGGAGGCCTAC AGTGAGATTGGGATGAAAGGCGAGCGCCGGAGGGGCAAGGGGCACGATGGCCTTTACCAGGGACTCAGTAC AGCCACCAAGGACACCTACGACGCCCTTCACATGCAGGCCCTGCCCCCTCGC (SEQ ID NO: 80). In some embodiments, the CD3ζ activation domain is encoded by SEQ ID NO: 80).

It is known that signals generated through the TCR alone are often insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs, which are known as immunoreceptor tyrosine-based activation motifs or ITAMs. In some embodiments, the ITAM contains a tyrosine separated from a leucine or an isoleucine by any two other amino acids (YxxL/I (SEQ ID NO: 139)). In some embodiments, the cytoplasmic domain contains 1, 2, 3, 4 or 5 ITAMs. An exemplary ITAM containing cytoplasmic domain is the CD3ζ activation domain. Further examples of ITAM containing primary cytoplasmic signaling sequences that can be used in the CARs of the instant disclosure include those derived from TCRζ, FcRγ, FcRβ, CD3γ, CD3δ, CD3ε, CD3ζ, CD5, CD22, CD79a, CD79b, and CD66d.

In some embodiments, the CD3ζ activation domain comprising a single ITAM comprises an amino acid sequence having at least 80% identity, at least 90% identity, at least 95% identity, at least 99% identity or is identical to a sequence of RVKFSRSADAPAYQQGQNQLYNELNLGRREEYDVLHMQALPPR (SEQ ID NO: 81). In some embodiments, the CD3ζ activation domain comprises SEQ ID NO: 81. In some embodiments, the CD3ζ activation domain comprising a single ITAM consists essentially of an amino acid sequence of RVKFSRSADAPAYQQGQNQLYNELNLGRREEYDVLHMQALPPR (SEQ ID NO: 81). In some embodiments, the CD3ζ activation domain comprising a single ITAM is encoded by a nucleotide sequence having at least 80% identity, at least 90% identity, at least 95% identity, at least 99% identity or is identical to a sequence of AGAGTGAAGT TCAGCAGGAG CGCAGACGCC CCCGCGTACC AGCAGGGCCA GAACCAGCTC TATAACGAGC TCAATCTAGG ACGAAGAGAG GAGTACGATG TTTTGCACAT GCAGGCCCTG CCCCCTCGC (SEQ ID NO: 82). In some embodiments, the CD3ζ activation domain is encoded by SEQ ID NO: 82.

In some embodiments, the cytoplasmic domain of the CAR can be designed to comprise the CD3ζ signaling domain by itself or combined with any other desired cytoplasmic domain(s) useful in the context of the CAR of the instant disclosure. For example, the cytoplasmic domain of the CAR can comprise a CD3ζ chain portion and a co-stimulatory domain. The co-stimulatory domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include the co-stimulatory domain is selected from the group consisting of IL-2Rβ, Fc Receptor gamma (FcRγ), Fc Receptor beta (FcRβ), CD3g molecule gamma (CD3γ), CD3δ, CD3ε, CD5 molecule (CD5), CD22 molecule (CD22), CD79a molecule (CD79a), CD79b molecule (CD79b), carcinoembryonic antigen related cell adhesion molecule 3 (CD66d), CD27 molecule (CD27), CD28 molecule (CD28), TNF receptor superfamily member 9 (4-1BB), TNF receptor superfamily member 4 (OX40), TNF receptor superfamily member 8 (CD30), CD40 molecule (CD40), programmed cell death 1 (PD-1), inducible T cell costimulatory (ICOS), lymphocyte function-associated antigen-1 (LFA-1), CD2 molecule (CD2), CD7 molecule (CD7), TNF superfamily member 14 (LIGHT), killer cell lectin like receptor C2 (NKG2C) and CD276 molecule (B7-H3) c-stimulatory domains, or functional fragments thereof. In some embodiments, the intracellular domains of CARs of the instant disclosure comprise at least one co-stimulatory domain. In some embodiments, the co-stimulatory domain is isolated or derived from CD28.

In some embodiments, the intracellular domains of CARs of the instant disclosure comprise at least one co-stimulatory domain. In some embodiments, the co-stimulatory domain is isolated or derived from CD28. In some embodiments, the CD28 co-stimulatory domain comprises an amino acid sequence having at least 80% identity, at least 90% identity, at least 95% identity, at least 99% identity or is identical to a sequence of RSKRSRLLHSDYMNMTPRRPGPTRKHYQPYAPPRDFAAYRS (SEQ ID NO: 83). In some embodiments, the CD28 co-stimulatory domain comprises or consists essentially of SEQ ID NO: 83). In some embodiments, the CD28 co-stimulatory domain is encoded by a nucleotide sequence having at least 80% identity, at least 90% identity, at least 95% identity, at least 99% identity or is identical to a sequence of

AGGAGCAAGCGGAGCAGACTGCTGCACAGCGACTACATGAACATGAC CCCCCGGAGGCCTGGCCCCACCCGGAAGCACTACCAGCCCTACGCCCCTCCC AGGGATTTCGCCGCCTACCGGAGC (SEQ ID NO: 84). In some embodiments, the CD28 co-stimulatory domain is encoded by SEQ ID NO: 84. The cytoplasmic domains within the cytoplasmic signaling portion of the CARs of the instant disclosure may be linked to each other in a random or specified order. Optionally, a short oligo- or polypeptide linker, for example between 2 and 10 amino acids in length may form the linkage. A glycine-serine doublet provides an example of a suitable linker.

### T Cell Receptors (TCRs)

The disclosure provides a first, activator receptor and immune cells comprising same. In some embodiments, the first receptor is a T cell receptor (TCR).

As used herein, a “TCR”, sometimes also called a “TCR complex” or “TCR/CD3 complex” refers to a protein complex comprising a TCR alpha chain, a TCR beta chain, and one or more of the invariant CD3 chains (zeta, gamma, delta and epsilon), sometimes referred to as subunits. The TCR alpha and beta chains can be disulfide-linked to function as a heterodimer to bind to peptide-WIC complexes. Once the TCR alpha/beta heterodimer engages peptide-WIC, conformational changes in the TCR complex in the associated invariant CD3 subunits are induced, which leads to their phosphorylation and association with downstream proteins, thereby transducing a primary stimulatory signal. In an exemplary TCR complex, the TCR alpha and TCR beta polypeptides form a heterodimer, CD3 epsilon and CD3 delta form a heterodimer, CD3 epsilon and CD3 gamma for a heterodimer, and two CD3 zeta form a homodimer.

Any suitable ligand binding domain may be fused to an extracellular domain, hinge domain or transmembrane of the TCRs described herein. For example, the ligand binding domain can be an antigen binding domain of an antibody or TCR, or comprise an antibody fragment, a Vβ only domain, a linear antibody, a single-chain variable fragment (scFv), or a single domain antibody (sdAb).

In some embodiments, the ligand binding domain is fused to one or more extracellular domains or transmembrane domains of one or more TCR subunits. The TCR subunit can be TCR alpha, TCR beta, CD3 delta, CD3 epsilon, CD3 gamma or CD3 zeta. For example, the ligand binding domain can be fused to TCR alpha, or TCR beta, or portions of the ligand binding can be fused to two subunits, for example portions of the ligand binding domain can be fused to both TCR alpha and TCR beta.

TCR subunits include TCR alpha, TCR beta, CD3 zeta, CD3 delta, CD3 gamma and CD3 epsilon. Any one or more of TCR alpha, TCR beta chain, CD3 gamma, CD3 delta, CD3 epsilon, or CD3 zeta, or fragments or derivative thereof, can be fused to one or more domains capable of providing a stimulatory signal of the disclosure, thereby enhancing TCR function and activity.

TCR transmembrane domains isolated or derived from any source are envisaged as within the scope of the disclosure. The transmembrane domain may be derived either from a natural or from a recombinant source. Where the source is natural, the domain may be derived from any membrane-bound or transmembrane protein.

In some embodiments, the transmembrane domain is capable of signaling to the intracellular domain(s) whenever the TCR complex has bound to a target. A transmembrane domain of particular use in this invention may include at least the transmembrane region(s) of e.g., the alpha, beta or zeta chain of the TCR, CD3 delta, CD3 epsilon or CD3 gamma, CD28, CD3 epsilon, CD45, CD4, CD5, CD8, CD9, CD16, CD22, CD33, CD37, CD64, CD80, CD86, CD134, CD137, CD154.

In some embodiments, the transmembrane domain can be attached to the extracellular region of a polypeptide of the TCR, e.g., the antigen binding domain of the TCR alpha or beta chain, via a hinge, e.g., a hinge from a human protein. For example, the hinge can be a human immunoglobulin (Ig) hinge, e.g., an IgG4 hinge, or a CD8a hinge. In some embodiments, the hinge is isolated or derived from CD8a or CD28.

In some embodiments, the extracellular ligand binding domain is attached to one or more transmembrane domains of the TCR. In some embodiments, the transmembrane domain comprises a TCR alpha transmembrane domain, a TCR beta transmembrane domain, or both. In some embodiments, the transmembrane comprises a CD3 zeta transmembrane domain.

A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 or up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 or up to 15 amino acids of the intracellular region).

In some embodiments, the transmembrane domain can be selected or modified by amino acid substitution to avoid binding of such domains to the transmembrane domains of the same or different surface membrane proteins, e.g., to minimize interactions with other members of the receptor complex.

When present, the transmembrane domain may be a natural TCR transmembrane domain, a natural transmembrane domain from a heterologous membrane protein, or an artificial transmembrane domain. The transmembrane domain may be a membrane anchor domain. Without limitation, a natural or artificial transmembrane domain may comprise a hydrophobic a-helix of about 20 amino acids, often with positive charges flanking the transmembrane segment. The transmembrane domain may have one transmembrane segment or more than one transmembrane segment. Prediction of transmembrane domains/segments may be made using publicly available prediction tools (e.g. TMHMM, Krogh et al. Journal of Molecular Biology 2001; 305(3):567-580; or TMpred, Hofmann & Stoffel Biol. Chem. Hoppe-Seyler 1993; 347: 166). Non-limiting examples of membrane anchor systems include platelet derived growth factor receptor (PDGFR) transmembrane domain, glycosylphosphatidylinositol (GPI) anchor (added post-translationally to a signal sequence) and the like.

In some embodiments, the transmembrane domain comprises a TCR alpha transmembrane domain. In some embodiments, the TCR alpha transmembrane domain comprises an amino acid sequence having at least 85% identity, at least 90% identity, at least 95% identity, at least 96% identity, at least 97% identity, at least 98% identity, at least 99% identity or is identical to a sequence of: VIGFRILLLKVAGFNLLMTLRLW (SEQ ID NO: 85). In some embodiments, the TCR alpha transmembrane domain comprises, or consists essentially of, SEQ ID NO: 85. In some embodiments, the TCR alpha transmembrane domain is encoded by a sequence of

(SEQ ID NO: 86)
GTGATTGGGTTCCGAATCCTCCTCCTGAAAGTGGCCGGG
TTTAATCTGCTCATGACGCTGCGGCTGTGG.

In some embodiments, the transmembrane domain comprises a TCR beta transmembrane domain. In some embodiments, the TCR beta transmembrane domain comprises an amino acid sequence having at least 85% identity, at least 90% identity, at least 95% identity, at least 96% identity, at least 97% identity, at least 98% identity, at least 99% identity or is identical to a sequence of: TILYEILLGKATLYAVLVSALVL (SEQ ID NO: 87). In some embodiments, the TCR beta transmembrane domain comprises, or consists essentially of, SEQ ID NO: 87. In some embodiments, the TCR beta transmembrane domain is encoded by a sequence of

(SEQ ID NO: 88)
ACCATCCTCTATGAGATCTTGCTAGGGAAGGCCACCT
TGTATGCCGTGCTGGTCAGTGCCCTCGTGCTG.

TCRs of the disclosure can comprise one or more intracellular domains. In some embodiments, the intracellular domain comprises one or more domains capable of providing a stimulatory signal to a transmembrane domain. In some embodiments, the intracellular domain comprises a first intracellular domain capable of providing a stimulatory signal and a second intracellular domain capable of providing a stimulatory signal. In other embodiments, the intracellular domain comprises a first, second and third intracellular domain capable of providing a stimulatory signal. The intracellular domains capable of providing a stimulatory signal are selected from the group consisting of a CD28 molecule (CD28) domain, a LCK proto-oncogene, Src family tyrosine kinase (Lck) domain, a TNF receptor superfamily member 9 (4-1BB) domain, a TNF receptor superfamily member 18 (GITR) domain, a CD4 molecule (CD4) domain, a CD8a molecule (CD8a) domain, a FYN proto-oncogene, Src family tyrosine kinase (Fyn) domain, a zeta chain of T cell receptor associated protein kinase 70 (ZAP70) domain, a linker for activation of T cells (LAT) domain, lymphocyte cytosolic protein 2 (SLP76) domain, (TCR) alpha, TCR beta, CD3 delta, CD3 gamma and CD3 epsilon intracellular domains.

In some embodiments, an intracellular domain comprises at least one intracellular signaling domain. An intracellular signaling domain generates a signal that promotes a function a cell, for example an immune effector function of a TCR containing cell, e.g., a TCR-expressing T-cell. In some embodiments, the intracellular domain of the first receptor of the disclosure includes at least one intracellular signaling domain. For example, the intracellular domains of CD3 gamma, delta or epsilon comprise signaling domains.

In some embodiments, the extracellular domain, transmembrane domain and intracellular domain are isolated or derived from the same protein, for example T-cell receptor (TCR) alpha, TCR beta, CD3 delta, CD3 gamma, CD3 epsilon or CD3 zeta.

Examples of intracellular domains for use in activator receptors of the disclosure include the cytoplasmic sequences of the TCR alpha, TCR beta, CD3 zeta, and 4-1BB, and the intracellular signaling co-receptors that act in concert to initiate signal transduction following antigen receptor engagement, as well as any derivative or variant of these sequences and any recombinant sequence that has the same functional capability.

In some embodiments, the intracellular signaling domain comprises a primary intracellular signaling domain. Exemplary primary intracellular signaling domains include those derived from the proteins responsible for primary stimulation, or antigen dependent stimulation.

In some embodiments, the intracellular domain comprises a CD3 delta intracellular domain, a CD3 epsilon intracellular domain, a CD3 gamma intracellular domain, a CD3 zeta intracellular domain, a TCR alpha intracellular domain or a TCR beta intracellular domain.

In some embodiments, the intracellular domain comprises a TCR alpha intracellular domain. In some embodiments, a TCR alpha intracellular domain comprises Ser-Ser. In some embodiments, a TCR alpha intracellular domain is encoded by a sequence of TCCAGC.

In some embodiments, the intracellular domain comprises a TCR beta intracellular domain. In some embodiments, the TCR beta intracellular domain comprises an amino acid sequence having at least 80% identity, at least 90% identity, or is identical to a sequence of: MAMVKRKDSR (SEQ ID NO: 89). In some embodiments, the TCR beta intracellular domain comprises, or consists essentially of SEQ ID NO: 89. In some embodiments, the TCR beta intracellular domain is encoded by a sequence of

(SEQ ID NO: 90)
ATGGCCATGGTCAAGAGAAAGGATTCCAGA.

In some embodiments, the intracellular signaling domain comprises at least one stimulatory intracellular domain. In some embodiments, the intracellular signaling domain comprises a primary intracellular signaling domain, such as a CD3 delta, CD3 gamma and CD3 epsilon intracellular domain, and one additional stimulatory intracellular domain, for example a co-stimulatory domain. In some embodiments, the intracellular signaling domain comprises a primary intracellular signaling domain, such as a CD3 delta, CD3 gamma and CD3 epsilon intracellular domain, and two additional stimulatory intracellular domains.

Exemplary co-stimulatory intracellular signaling domains include those derived from proteins responsible for co-stimulatory signals, or antigen independent stimulation. Co-stimulatory molecules include, but are not limited to an MHC class I molecule, BTLA, a Toll ligand receptor, as well as DAP10, DAP12, CD30, LIGHT, OX40, CD2, CD27, CDS, ICAM-1, LFA-1 (CD11a/CD18) 4-1BB (CD137, TNF receptor superfamily member 9), and CD28 molecule (CD28). A co-stimulatory protein can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, a ligand that specifically binds with CD83, CD4, and the like. The co-stimulatory domain can comprise the entire intracellular portion, or the entire native intracellular signaling domain, of the molecule from which it is derived, or a functional fragment thereof.

In some embodiments, the stimulatory domain comprises a co-stimulatory domain. In some embodiments, the co-stimulatory domain comprises a CD28 or 4-1BB co-stimulatory domain. CD28 and 4-1BB are well characterized co-stimulatory molecules required for full T cell activation and known to enhance T cell effector function. For example, CD28 and 4-1BB have been utilized in chimeric antigen receptors (CARs) to boost cytokine release, cytolytic function, and persistence over the first-generation CAR containing only the CD3 zeta signaling domain. Likewise, inclusion of co-stimulatory domains, for example CD28 and 4-1BB domains, in TCRs can increase T cell effector function and specifically allow co-stimulation in the absence of co-stimulatory ligand, which is typically down-regulated on the surface of tumor cells. In some embodiments, the stimulatory domain comprises a CD28 intracellular domain or a 4-1BB intracellular domain.

### Inhibitory Receptors

The disclosure provides a second receptor, comprising an extracellular ligand binding domain specific to a non-target antigen selected from the group of proteins listed in table 4, or a peptide antigen of any of these proteins in a complex with a major histocompatibility complex class I (MHC-I), and immune cells comprising same. In some embodiments, the second receptor is an inhibitory chimeric antigen receptor (iCAR).

In some embodiments, the second receptor is humanized.

The disclosure provides a second receptor, which is an inhibitory receptor, comprising an extracellular ligand binding that can bind a non-target antigen that is differentially expressed between cancer cells and healthy cells. This ability to discriminate between differential expression of a non-target antigen allows the second receptor to inhibit activation of immune cells comprising the second receptor in the presence of non-target cells that express the non-target antigen recognized by the ligand binding domain of the second receptor. However, activation of immune cells is not inhibited in the presence of target cells that have low or no expression of the non-target antigen, for example cancer cells.

### Inhibitor Targets

Activation of the inhibitory receptor is mediated by the presence of the non-target antigen on the surface of a cell. A cell that expresses the non-target antigen will activate the inhibitory receptor based on the level of expression of the non-target antigen. In some embodiments, the non-target antigen is expressed by both target and non-target cells. However, in these embodiments, the non-target antigen is expressed by non-target cells at a higher level than the target cells. The higher levels of non-target antigen expressed by the non-target cells activate the inhibitory receptor, thereby preventing activation of the immune cell. In contrast, the lower levels of non-target antigen expressed by the target are not sufficient to activate the inhibitory receptor, leading to activation of the immune cell.

In alternative embodiments, the non-target antigen is expressed by non-target cells but not by target cells. In the absence of expression of the non-target antigen, the target cells activate the target receptor, thereby activating the immune cells.

Differential expression can be determined by any techniques known in the art used to measure expression. These include, inter alia, techniques for measuring mRNA and/or protein levels of a target gene in a cell. Methods of measuring protein levels in samples include immunohistochemistry, enzyme-linked immunosorbent assays (ELISA), and analytical methods such as liquid chromatography-mass spectrometry (LC-MS). Methods of measuring mRNA levels include real time quantitative reverse transcription PCR (qRT-PCR), as well as high throughput sequencing. Expression differences can be observed between, for example, a normal cell and a diseased cell, for example a cancer cell.

Activation of the inhibitory receptor by a non-target antigen can occur according to various modalities known in the art. Activation of the inhibitory receptor by a non-target antigen can be determined by methods known in the art. For example, the level of downstream intracellular signaling in a cell expressing the inhibitory receptor can be measured through the use of a reporter gene.

Without wishing to be bound by theory, whether or not expression of a non-target antigen inhibits activation of an immune cell via activation of the inhibitory receptor can occur according to the ratio of the non-target antigen to the inhibitor receptor. The expression levels of the non-target antigen and the inhibitory receptor, and the ratio thereof, can be determined by methods known in the art, including, inter alia, immunohistochemistry and fluorescence activated cell sorting (FACS). Analysis of the expression levels of the non-target antigen on target and non-target cells can be used to predict selective targeting of the immune cells expressing the inhibitory receptor. Low or no expression of the non-target antigen on a target or non-target cell can indicate, for example, that the inhibitory receptor will not be activated in an immune cell of the disclosure.

Alternatively, or in addition, and without wishing to be bound by theory, inhibition of immune cell activation by a non-target antigen via activation of the inhibitory receptor can depend on the affinity of the non-target antigen for the inhibitory receptor. Methods of measuring affinity are known in the art, and include, inter alia, enzyme-linked immunosorbent assay or radioimmunoassay methods.

Alternatively, or in addition, and without wishing to be bound by theory, inhibition of immune cell activation by a non-target antigen via activation of the inhibitory receptor can occur according to cross talk between the inhibitory receptor and the activator receptor, leading to down-regulation of the activity of the activator receptor. For example, activation of the inhibitory receptor by the non-target antigen can lead to reduced expression of the activator receptor on the surface of the immune cell.

In some embodiments, the non-target antigen is expressed at a lower level in a target cell than a normal cell. In some embodiments, the non-target antigen is expressed by healthy cells, i.e. cells that are not cancer cells. In some embodiments, the non-target antigen expression level is at least about 10 times less, at least about 30 times less, at least about 50 times less, at least about 70 times less, at least about 90 times less, at least about 100 times less, at least about 110 times less, at least about 150 times less, at least about 200 times less, at least about 250 times less, at least about 300 times less, at least about 350 times less, at least about 400 times less, at least about 450 times less, at least about 500 times less, at least about 600 times less, at least about 700 times less, at least about 800 times less, at least about 900 times less or at least about 1000 times less in the target cell than in the non-target cell. In some embodiments, the non-target antigen expression level is about 10 times less, about 30 times less, about 50 times less, about 70 times less, about 90 times less, about 100 times less, or about 110 times less than the plurality of healthy cells. In some embodiments, the non-target antigen expression level is at least about 5 times less in the plurality of cancer cells than in the plurality of healthy cells. In some embodiments, the non-target antigen expression level is at least about 5 times less in a target cell than a non-target cell. In some embodiments, the target cells are a plurality of cancer cells that have low or no expression of the non-target antigen.

Any cell surface molecule expressed by the non-target cells that is not expressed by target cells may be a suitable non-target antigen for the second receptor extracellular ligand binding domain. For example, a cell adhesion molecule, a cell-cell signaling molecule, an extracellular domain, a molecule involved in chemotaxis, a glycoprotein, a G protein-coupled receptor, a transmembrane protein, a receptor for a neurotransmitter or a voltage gated ion channel can be used as a non-target antigen.

In some embodiments, the non-target antigen is selected from the group consisting of BEST2, BEST4, SCARA5, EPHA7, and TGFBR2, or a peptide antigen of any of these in a complex with a major histocompatibility complex class I (MHC-I). In some embodiments, the non-target antigen is BEST2 or a peptide antigen thereof in a complex with MHC-I. In some embodiments, the non-target antigen is BEST4 or a peptide antigen thereof in a complex with MHC-I. In some embodiments, the non-target antigen is SCARA5 or a peptide antigen thereof in a complex with MHC-I. In some embodiments, the non-target antigen is EPHA7 or a peptide antigen thereof in a complex with MHC-I. In some embodiments, the non-target antigen is TGFBR2 or a peptide antigen thereof in a complex with MHC-I.

In some embodiments, the target antigen is a peptide antigen of a cancer cell-specific antigen in a complex with a major histocompatibility complex class I (MHC-I).

Non-target MHC-I (pMHC) antigens comprising any of HLA-A, HLA-B or HLA-C are envisaged as within the scope of the disclosure. In some embodiments, the non-target antigen comprises HLA-A. In some embodiments, the non-target antigen comprises a human leukocyte antigen A*02 allele (HLA-A*02). In some embodiments, the non-target antigen comprises HLA-B. In some embodiments, the non-target antigen comprises HLA-C.

Non-target antigens comprise proteins that have low or no expression in cancer cells, for example colorectal cancer cells, but are expressed in normal tissues, such as normal colorectal tissue.

In some embodiments, the non-target antigen comprises bestrophin-2 (BEST2) or an antigen peptide thereof in a complex with MHC-I. A human BEST2 is described in NCBI record number NP_060152.2, the contents of which are incorporated by reference herein in their entirety. In some embodiments, BEST2 comprises an amino acid sequence of:

(SEQ ID NO: 91)

| | |
|---|---|
| 1 | MTVTYTARVA NARFGGFSQL LLLWRGSTYK |
| | LLWRELLCFL GFYMALSAAY RFVLTEGQKR |
| 61 | YFEKLVIYCD QYASLIPVSF VLGFYVTLVV |
| | NRWWSQYLCM PLPDALMCVV AGTVHGRDDR |
| 121 | GRLYRRTLMR YAGLSAVLIL RSVSTAVFKR |
| | FPTIDHVVEA GFMTREERKK FENLNSSYNK |
| 181 | YWVPCVWFSN LAAQARREGR IRDNSALKLL |
| | LEELNVFRGK CGMLFHYDWI SVPLVYTQVV |
| 241 | TIALYSYFLA CLIGRQFLDP AQGYKDHDLD |
| | LCVPIFTLLQ FFFYAGWLKV AEQLINPFGE |
| 301 | DDDDFETNFL IDRNFQVSML AVDEMYDDLA |
| | VLEKDLYWDA AEARAPYTAA TVFQLRQPSF |
| 361 | QGSTFDITLA KEDMQFQRLD GLDGPMGEAP |
| | GDFLQRLLPA GAGMVAGGPL GRRLSFLLRK |
| 421 | NSCVSEASTG ASCSCAVVPE GAAPECSCGD |
| | PLLDPGLPEP EAPPPAGPEP LTLIPGPVEP |
| 481 | FSIVTMPGPR GPAPPWLPSP IGEEEENLA. |

In some embodiments, BEST2 comprises a sequence that shares at least 80%, at least 85%, at least 90%, at least 95%, at least 96%, at least 97%, at least 98%, or at least 99% identity to SEQ ID NO: 91.

In some embodiments, the non-target antigen comprises bestrophin-4 (BEST4) or an antigen peptide thereof in a complex with MHC-I. A human BEST4 is described in NCBI record number NP_695006, the contents of which are incorporated by reference herein in their entirety. In some embodiments, BEST4 comprises an amino acid sequence of:

| (SEQ ID NO: 92) | |
|---|---|
| 1 | MTVSYTLKVA EARFGGFSGL LLRWRGSIYK |
| | LLYKEFLLFG ALYAVLSITY RLLLTQEQRY |
| 61 | VYAQVARYCN RSADLIPLSF VLGFYVTLW |
| | NRWWSQYTSI PLPDQLMCVI SASVHGVDQR |
| 121 | GRLLRRTLIR YANLASVLVL RSVSTRVLKR |
| | FPTMEHVVDA GFMSQEERKK FESLKSDFNK |
| 181 | YWVPCVWFTN LAAQARRDGR IRDDIALCLL |
| | LEELNKYRAK CSMLFHYDWI SIPLVYTQVV |
| 241 | TIAVYSFFAL SLVGRQFVEP EAGAAKPQKL |
| | LKPGQEPAPA LGDPDMYVPL TTLLQFFFYA |
| 301 | GWLKVAEQII NPFGEDDDDF ETNQLIDRNL |
| | QVSLLSVDEM YQNLPPAEKD QYWDEDQPQP |
| 361 | PYTVATAAES LRPSFLGSTF NLRMSDDPEQ |
| | SLQVEASPGS GRPAPAAQTP LLGRFLGVGA |
| 421 | PSPAISLRNF GRVRGTPRPP HLLRFRAEEG |
| | GDPEAAARIE EESAESGDEA LEP. |

In some embodiments, BEST4 comprises a sequence that shares at least 80%, at least 85%, at least 90%, at least 95%, at least 96%, at least 97%, at least 98%, or at least 99% identity to SEQ ID NO: 92.

In some embodiments, the non-target antigen comprises scavenger receptor class A member 5 (SCARA5) or an antigen peptide thereof in a complex with MHC-I. A human SCARA5 is described in NCBI record number NP_776194, the contents of which are incorporated by reference herein in their entirety. In some embodiments, SCARA5 comprises an amino acid sequence of:

(SEQ ID NO: 93)

| | |
|---|---|
| 1 | MENKAMYLHT VSDCDTSSIC EDSFDGRSLS |
| | KLNLCEDGPC HKRRASICCT QLGSLSALKH |
| 61 | AVLGLYLLVF LILVGIFILA VSRPRSSPDD |
| | LKALTRNVNR LNESFRDLQL RLLQAPLQAD |
| 121 | LTEQVWKVQD ALQNQSDSLL ALAGAVQRLE |
| | GALWGLQAQA VQTEQAVALL RDRTGQQSDT |
| 181 | AQLELYQLQV ESNSSQLLLR RHAGLLDGLA |
| | RRVGILGEEL ADVGGVLRGL NHSLSYDVAL |
| 241 | HRTRLQDLRV LVSNASEDTR RLRLAHVGME |
| | LQLKQELAML NAVTEDLRLK DWEHSIALRN |
| 301 | ISLAKGPPGP KGDQGDEGKE GRPGIPGLPG |
| | LRGLPGERGT PGLPGPKGDD GKLGATGPMG |
| 361 | MRGFKGDRGP KGEKGEKGDR AGDASGVEAP |
| | MMIRLVNGSG PHEGRVEVYH DRRWGTVCDD |
| 421 | GWDKKDGDVV CRMLGFRGVE EVYRTARFGQ |
| | GTGRIWMDDV ACKGTEETIF RCSFSKWGVT |
| 481 | NCGHAEDASV TCNRH. |

In some embodiments, SCARA5 comprises a sequence that shares at least 80%, at least 85%, at least 90%, at least 95%, at least 96%, at least 97%, at least 98%, or at least 99% identity to SEQ ID NO: 93.

In some embodiments, the non-target antigen comprises ephrin type-A receptor 7 (EPHA7), or an antigen peptide thereof in a complex with MHC-I. A human EPHA7 is described in NCBI record number NP_004431, the contents of which are incorporated by reference herein in their entirety. In some embodiments, EPHA7 isoform 1 precursor comprises an amino acid sequence of:

(SEQ ID NO: 94)

| | |
|---|---|
| 1 | MVFQTRYPSW IILCYIWLLR FAHTGEAQAA |
| | KEVLLLDSKA QQTELEWISS PPNGWEEISG |
| 61 | LDENYTPIRT YQVCQVMEPN QNNWLRTNWI |
| | SKGNAQRIFV ELKFTLRDCN SLPGVLGTCK |
| 121 | ETFNLYYYET DYDTGRNIRE NLYVKIDTIA |
| | ADESFTQGDL GERKMKLNTE VREIGPLSKK |
| 181 | GFYLAFQDVG ACIALVSVKV YYKKCWSIIE |
| | NLAIFPDTVT GSEFSSLVEV RGTCVSSAEE |
| 241 | EAENAPRMHC SAEGEWLVPI GKCICKAGYQ |
| | QKGDTCEPCG RGFYKSSSQD LQCSRCPTHS |
| 301 | FSDKEGSSRC ECEDGYYRAP SDPPYVACTR |
| | PPSAPQNLIF NINQTTVSLE WSPPADNGGR |
| 361 | NDVTYRILCK RCSWEQGECV PCGSNIGYMP |
| | QQTGLEDNYV TVMDLLAHAN YTFEVEAVNG |
| 421 | VSDLSRSQRL FAAVSITTGQ AAPSQVSGVM |
| | KERVLQRSVE LSWQEPEHPN GVITEYEIKY |
| 481 | YEKDORERTY STVKTKSTSA SINNLKPGTV |
| | YVFQIRAFTA AGYGNYSPRL DVATLEEATG |
| 541 | KMFEATAVSS EQNPVIIIAV VAVAGTIILV |
| | FMVFGFIIGR RHCGYSKADQ EGDEELYFHF |
| 601 | KFPGTKTYID PETYEDPNRA VHQFAKELDA |
| | SCIKIERVIG AGEFGEVCSG RLKLPGKRDV |
| 661 | AVAIKTLKVG YTEKQRRDFL CEASIMGQFD |
| | HPNVVHLEGV VTRGKPVMIV IEFMENGALD |
| 721 | AFLRKHDGQF TVIQLVGMLR GIAAGMRYLA |
| | DMGYVHRDLA ARNILVNSNL VCKVSDFGLS |
| 781 | RVIEDDPEAV YTTTGGKIPV RWTAPEAIQY |
| | RKFTSASDVW SYGIVMWEVM SYGERPYWDM |
| 841 | SNQDVIKAIE EGYRLPAPMD CPAGLHQLML |
| | DCWQKERAER PKFEQIVGIL DKMIRNPNSL |
| 901 | KTPLGTCSRP ISPLLDQNTP DFTTFCSVGE |
| | WLQAIKMERY KDNFTAAGYN SLESVARMTI |
| 961 | EDVMSLGITL VGHQKKIMSS IQTMRAQMLH |
| | LHGTGIQV. |

In some embodiments, EPHA7 comprises a sequence that shares at least 80%, at least 85%, at least 90%, at least 95%, at least 96%, at least 97%, at least 98%, or at least 99% identity to SEQ ID NO: 94.

In some embodiments, the non-target antigen comprises transforming growth factor beta receptor 2 (TGFBR2), or an antigen peptide thereof in a complex with MHC-I. A human TGFBR2 isoform A precursor is described in NCBI record number NP_001020018.1, the contents of which are incorporated by reference herein in their entirety. In some embodiments, TGFBR2 isoform A precursor comprises an amino acid sequence of:

(SEQ ID NO: 95)

| | |
|---|---|
| 1 | MGRGLLRGLW PLHIVLWTRI ASTIPPHVQK |
| | SDVEMEAQKD EIICPSCNRT AHPLRHINND |
| 61 | MIVTDNNGAV KFPQLCKFCD VRFSTCDNQK |
| | SCMSNCSITS ICEKPQEVCV AVWRKNDENI |
| 121 | TLETVCHDPK LPYHDFILED AASPKCIMKE |
| | KKKPGETFFM CSCSSDECND NIIFSEEYNT |
| 181 | SNPDLLLVIF QVTGISLLPP LGVAISVIII |
| | FYCYRVNRQQ KLSSTWETGK TRKLMEFSEH |
| 241 | CAIILEDDRS DISSTCANNI NHNTELLPIE |
| | LDTLVGKGRF AEVYKAKLKQ NTSEQFETVA |
| 301 | VKIFPYEEYA SWKTEKDIFS DINLKHENIL |
| | QFLTAEERKT ELGKQYWLIT AFHAKGNLQE |
| 361 | YLTRHVISWE DLRKLGSSLA RGIAHLHSDH |
| | TPCGRPKMPI VHRDLKSSNI LVKNDLTCCL |
| 421 | CDFGLSLRLD PTLSVDDLAN SGQVGTARYM |
| | APEVLESRMN LENVESFKQT DVYSMALVLW |
| 481 | EMTSRCNAVG EVKDYEPPFG SKVREHPCVE |
| | SMKDNVLRDR GRPEIPSFWL NHQGIQMVCE |
| 541 | TLTECWDHDP EARLTAQCVA ERFSELEHLD |
| | RLSGRSCSEE KIPEDGSLNT TK. |

A human TGFBR2 isoform B precursor is described in NCBI record number NP_003233.4, the contents of which are incorporated by reference herein in their entirety. In some embodiments, TGFBR2 isoform B precursor comprises an amino acid sequence of:

(SEQ ID NO: 96)

| | |
|---|---|
| 1 | MGRGLLRGLW PLHIVLWTRI ASTIPPHVQK |
| | SVNNDMIVTD NNGAVKFPQL CKFCDVRFST |
| 61 | CDNQKSCMSN CSITSICEKP QEVCVAVWRK |
| | NDENITLETV CHDPKLPYHD FILEDAASPK |
| 121 | CIMKEKKKPG ETFFMCSCSS DECNDNIIFS |
| | EEYNTSNPDL LLVIFQVTGI SLLPPLGVAI |
| 181 | SVIIIFYCYR VNRQQKLSST WETGKTRKLM |
| | EFSEHCAIIL EDDRSDISST CANNINHNTE |
| 241 | LLPIELDTLV GKGRFAEVYK AKLKQNTSEQ |
| | FETVAVKIFP YEEYASWKTE KDIFSDINLK |
| 301 | HENILQFLTA EERKTELGKQ YWLITAFHAK |
| | GNLQEYLTRH VISWEDLRKL GSSLARGIAH |
| 361 | LHSDHTPCGR PKMPIVHRDL KSSNILVKND |
| | LTCCLCDFGL SLRLDPTLSV DDLANSGQVG |
| 421 | TARYMAPEVL ESRMNLENVE SFKQTDVYSM |
| | ALVLWEMTSR CNAVGEVKDY EPPFGSKVRE |
| 481 | HPCVESMKDN VLRDRGRPEI PSFWLNHQGI |
| | QMVCETLTEC WDHDPEARLT AQCVAERFSE |
| 541 | LEHLDRLSGR SCSEEKIPED GSLNTTK. |

In some embodiments, TGFBR2 comprises a sequence that shares at least 80%, at least 85%, at least 90%, at least 95%, at least 96%, at least 97%, at least 98%, or at least 99% identity to SEQ ID NO: 95 or SEQ ID NO: 96.

Inhibitory Chimeric Antigen Receptors (iCARs)

The disclosure provides a second receptor that is an inhibitory chimeric antigen receptor (iCAR). The inhibitory CAR may comprise an extracellular ligand binding domain that binds to and recognizes the non-target antigen or a peptide derivative thereof in a MHC-I complex.

The term “inhibitory chimeric antigen receptor” or “iCAR” as used herein refers to an antigen-binding domain that is fused to an intracellular signaling domain capable of transducing an inhibitory signal that inhibits or suppresses the immune activity of an immune cell. iCARs have immune cell inhibitory potential, and are distinct and distinguishable from CARs, which are receptors with immune cell activating potential. For example, CARs are activating receptors as they include intracellular stimulatory and/or co-stimulatory domains. iCARs are inhibiting receptors that contain intracellular inhibitory domains.

As used herein “inhibitory signal” refers to signal transduction or changes in protein expression in an immune cell resulting in suppression of an immune response (e.g., decrease in cytokine production). Inhibition or suppression of an immune cell can selective and/or reversible, or not selective and/or reversible.

iCARs of the disclosure may comprise an extracellular ligand binding domain. Any type of ligand binding domain that can regulate the activity of a receptor in a ligand dependent manner is envisaged as within the scope of the instant disclosure.

In some embodiments, the ligand binding domain is an antigen binding domain. Exemplary antigen binding domains include, inter alia, ScFv, SdAb, Vβ-only domains, and TCR antigen binding domains derived from the TCR α and β chain variable domains.

Any type of antigen binding domain is envisaged as within the scope of the instant disclosure.

In some embodiments, the extracellular ligand binding domain of the second receptor binds to and recognizes any one of BEST2, BEST4, SCARA5, EPHA7, or TGFBR2, or an antigen peptide of any of these in a complex with a major histocompatibility complex class I (MHC-I). In some embodiments, the extracellular ligand binding domain of the second receptor is an ScFv.

In some embodiments, the extracellular ligand binding domain of the second receptor is fused to the extracellular domain of an iCAR.

In some embodiments, the iCARs of the present disclosure comprise an extracellular hinge region. Exemplary hinges can be isolated or derived from IgD and CD8 domains, for example IgG1. In some embodiments, the hinge is isolated or derived from CD8a or CD28.

The iCARs of the present disclosure can be designed to comprise a transmembrane domain that is fused to the extracellular domain of the iCAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions may be isolated or derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, or from an immunoglobulin such as IgG4. Alternatively the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the intracellular domain of the iCAR. A glycine-serine doublet provides a particularly suitable linker.

The disclosure provides an iCAR comprising an intracellular domain. The intracellular domain of the iCARs of the instant invention is responsible for inhibiting activation of the immune cells comprising the iCAR, which would otherwise be activated in response to activation signals by the first receptor. In some embodiments, the inhibitory intracellular domain comprises an immunoreceptor tyrosine-based inhibitory motif (ITIM). In some embodiments, the inhibitory intracellular domain comprising an ITIM can be isolated or derived from an immune checkpoint inhibitor such as CTLA-4 and PD-1. CTLA-4 and PD-1 are immune inhibitory receptors expressed on the surface of T cells, and play a pivotal role in attenuating or terminating T cell responses.

In some embodiments, an inhibitory intracellular domain is isolated from human tumor necrosis factor related apoptosis inducing ligand (TRAIL) receptor and CD200 receptor 1. In some embodiments, the TRAIL receptor comprises TR10A, TR10B or TR10D.

In some embodiments, an inhibitory intracellular domain is isolated from phosphoprotein membrane anchor with glycosphingolipid microdomains 1 (PAG1). In some embodiments, an inhibitory intracellular domain is isolated from leukocyte immunoglobulin like receptor B1 (LILRB1).

In some embodiments, the inhibitory domain is isolated or derived from a human protein, for example a human TRAIL receptor, CTLA-4, PD-1, PAG1 or LILRB1 protein.

In some embodiments, the inhibitory domain comprises an intracellular domain, a transmembrane or a combination thereof. In some embodiments, the inhibitory domain comprises an intracellular domain, a transmembrane domain, a hinge region or a combination thereof.

In some embodiments, the inhibitory domain is isolated or derived from killer cell immunoglobulin like receptor, three Ig domains and long cytoplasmic tail 2 (KIR3DL2), killer cell immunoglobulin like receptor, three Ig domains and long cytoplasmic tail 3 (KIR3DL3), leukocyte immunoglobulin like receptor B1 (LIR1, also called LIR-1 and LILRB1), programmed cell death 1 (PD-1), Fc gamma receptor IIB (FcgRIIB), killer cell lectin like receptor K1 (NKG2D), CTLA-4, a domain containing a synthetic consensus ITIM, a ZAP70 SH2 domain (e.g., one or both of the N and C terminal SH2 domains), or ZAP70 KI_K369A (kinase inactive ZAP70).

In some embodiments, the inhibitory domain is isolated or derived from a human protein.

In some embodiments, the second, inhibitory receptor comprises an inhibitory domain. In some embodiments, the second, inhibitory receptor comprises an inhibitory intracellular domain and/or an inhibitory transmembrane domain. In some embodiments, the inhibitory intracellular domain is fused to an intracellular domain of an iCAR. In some embodiments, the inhibitory intracellular domain is fused to the transmembrane domain of an iCAR.

### Polynucleotides and Vectors

The disclosure provides polynucleotides encoding the sequence(s) of the first and second receptors of the disclosure. The disclosure provides immune cells comprising the polynucleotides and vectors described herein.

In some embodiments, the sequence of the first and/or second receptor is operably linked to a promoter. In some embodiments, the sequence encoding the first receptor is operably linked to a first promoter, and the sequence encoding the second receptor is operably linked to a second promoter.

The disclosure provides vectors comprising the polynucleotides described herein.

In some embodiments, the first receptor is encoded by a first vector and the second receptor is encoded by second vector. In some embodiments, both receptors are encoded by a single vector.

In some embodiments, the first and second receptors are encoded by a single vector. Methods of encoding multiple polypeptides using a single vector will be known to persons of ordinary skill in the art, and include, inter alia, encoding multiple polypeptides under control of different promoters, or, if a single promoter is used to control transcription of multiple polypeptides, use of sequences encoding internal ribosome entry sites (IRES) and/or self-cleaving peptides. Exemplary self-cleaving peptides include T2A, P2A, E2A and F2A self-cleaving peptides. In some embodiments, the T2A self-cleaving peptide comprises a sequence of EGRGSLLTCGDVEENPGP (SEQ ID NO: 33). In some embodiments, the P2A self-cleaving peptide comprises a sequence of ATNFSLLKQAGDVEENPGP (SEQ ID NO: 32). In some embodiments, the E2A self-cleaving peptide comprises a sequence of QCTNYALLKLAGDVESNPGP (SEQ ID NO: 34). In some embodiments, the F2A self-cleaving peptide comprises a sequence of VKQTLNFDLLKLAGDVESNPGP (SEQ ID NO: 35).

In some embodiments, the vector is an expression vector, i.e. for the expression of the first and/or second receptor in a suitable cell.

Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

The expression of natural or synthetic nucleic acids encoding receptors is typically achieved by operably linking a nucleic acid encoding the receptor or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The polynucleotides encoding the receptors can be cloned into a number of types of vectors. For example, the polynucleotides can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to cells, such as immune cells, in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 basepairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a receptor, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected or transduced cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). One method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Immune Cells

The disclosure provides immune cells comprising the receptors, vectors and polynucleotides described herein. In some embodiments, the immune cells comprise: (a) a first receptor, optionally a chimeric antigen receptor (CAR) or T cell receptor (TCR), comprising an extracellular ligand binding domain specific to a target antigen selected from: (i) a cancer cell-specific antigen, or a peptide antigen thereof in a complex with a major histocompatibility complex class I (MHC-I); or (ii) CEA cell adhesion molecule 5 (CEA), or a peptide antigen thereof in a complex with a major histocompatibility complex class I (MHC-I); and (b) a second receptor, optionally an inhibitory chimeric antigen receptor (iCAR), comprising an extracellular ligand binding domain specific to a non-target antigen selected from the group consisting of BEST2, BEST4, SCARA5, EPHA7 and TGFBR2, or an antigen peptide thereof in a complex with a major histocompatibility complex class I (MHC-I), wherein the non-target antigen is expressed at a lower level by a population of target cells than by a population of non-target cells. In some embodiments, the first receptor is a CAR or TCR. In some embodiments, the second receptor is an iCAR.

As used herein, the term “immune cell” refers to a cell involved in the innate or adaptive (acquired) immune systems. Exemplary innate immune cells include phagocytic cells such as neutrophils, monocytes and macrophages, Natural Killer (NK) cells, polymophonuclear leukocytes such as neutrophils eosinophils and basophils and mononuclear cells such as monocytes, macrophages and mast cells. Immune cells with roles in acquired immunity include lymphocytes such as T-cells and B-cells.

As used herein, a “T-cell” refers to a type of lymphocyte that originates from a bone marrow precursor that develops in the thymus gland. There are several distinct types of T-cells, which develop upon migration to the thymus, which include helper CD4+ T-cells, cytotoxic CD8+ T cells, memory T cells, regulatory CD4+ T-cells and stem memory T-cells. Different types of T-cells can be distinguished by the ordinarily skilled artisan based on their expression of markers. Methods of distinguishing between T-cell types will be readily apparent to the ordinarily skilled artisan.

In some embodiments, the first receptor and the second receptor together specifically activate the immune cell in the presence of the target cell.

In some embodiments, the immune cell is selected form the group consisting of T cells, B cells and Natural Killer (NK) cells. In some embodiments, the immune cell is a T cell, for example a CD8+CD4− T cell.

In some embodiments, the immune cell is non-natural. In some embodiments, the immune cell is isolated.

Methods transforming populations of immune cells, such as T cells, with the vectors of the instant disclosure will be readily apparent to the person of ordinary skill in the art. For example, CD3+ T cells can be isolated from PBMCs using a CD3+ T cell negative isolation kit (Miltenyi), according to manufacturer's instructions. T cells can be cultured at a density of 1×10\hat{ }6 cells/mL in X-Vivo 15 media supplemented with 5% human AB serum and 1% Pen/strep in the presence of CD3/28 Dynabeads (1:1 cell to bead ratio) and 300 Units/mL of IL-2 (Miltenyi). After 2 days, T cells can be transduced with viral vectors, such as lentiviral vectors using methods known in the art. In some embodiments, the viral vector is transduced at a multiplicity of infection (MOI) of 5. Cells can then be cultured in IL-2 or other cytokines such as combinations of IL-7/15/21 for an additional 5 days prior to enrichment. Methods of isolating and culturing other populations of immune cells, such as B cells, or other populations of T cells, will be readily apparent to the person of ordinary skill in the art. Although this method outlines a potential approach it should be noted that these methodologies are rapidly evolving. For example excellent viral transduction of peripheral blood mononuclear cells can be achieved after 5 days of growth to generate a >99% CD3+ highly transduced cell population.

Methods of activating and culturing populations of T cells comprising the TCRs, CARs, iCARs, receptors or vectors encoding same, will be readily apparent to the person of ordinary skill in the art.

Whether prior to or after genetic modification of T cells to express a TCR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041, 10040846; and U.S. Pat. Appl. Pub. No. 2006/0121005.

In some embodiments, T cells of the instant disclosure are expanded and activated in vitro. Generally, the T cells of the instant disclosure are expanded in vitro by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In some embodiments, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4+ T cell expansion and T cell growth is used. In some embodiments, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. In some embodiments, a ratio of 1:1 cells to beads is used. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached to contact the T cells. In one embodiment the cells (for example, CD4+ T cells) and beads (for example, DYNABEADS CD3/CD28 T paramagnetic beads at a ratio of 1:1) are combined in a buffer. Again, those of ordinary skill in the art can readily appreciate any cell concentration may be used. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. In some embodiments, cells that are cultured at a density of 1×10⁶ cells/mL are used.

In some embodiments, the mixture may be cultured for several hours (about 3 hours) to about 14 days or any hourly integer value in between. In another embodiment, the beads and T cells are cultured together for 2-3 days. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. In some embodiments, the media comprises X-VIVO-15 media supplemented with 5% human AB serum, 1% penicillin/streptomycin (pen/strep) and 300 Units/ml of IL-2 (Miltenyi).

The T cells are maintained under conditions necessary to support growth, for example, an appropriate temperature (e.g., 37° C.) and atmosphere (e.g., air plus 5% CO2).

In some embodiments, the T cells comprising TCRs, CARs and iCARS of the disclosure are autologous. Prior to expansion and genetic modification, a source of T cells is obtained from a subject. Immune cells such as T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as Ficoll™ separation.

In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In alternative embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca2+-free, Mg2+-free PBS, PlasmaLyte A, or other saline solution with or without buffer. Alternatively, the undesirable components of the apheresis sample may be removed and the cells directly resuspended in culture media.

In some embodiments, immune cells such as T cells are isolated from peripheral blood lymphocytes by lysing the red blood cells and depleting the monocytes, for example, by centrifugation through a PERCOLL™ gradient or by counterflow centrifugal elutriation. Specific subpopulations of immune cells, such as T cells, B cells, or CD4+ T cells can be further isolated by positive or negative selection techniques. For example, in one embodiment, T cells are isolated by incubation with anti-CD4-conjugated beads, for a time period sufficient for positive selection of the desired T cells.

Enrichment of an immune cell population, such as a T cell population, by negative selection can be accomplished with a combination of antibodies directed to surface markers unique to the negatively selected cells. One method is cell sorting and/or selection via negative magnetic immune-adherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD 14, CD20, CD 11b, CD 16, HLA-DR, and CD8.

For isolation of a desired population of immune cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads.

In some embodiments, the cells may be incubated on a rotator for varying lengths of time at varying speeds at either 2-10° C. or at room temperature.

T cells for stimulation, or PBMCs from which immune cells such as T cells are isolated, can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

### Pharmaceutical Compositions

The disclosure provides pharmaceutical compositions comprising immune cells comprising the first and second receptors of the disclosure and a pharmaceutically acceptable diluent, carrier or excipient.

Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; and preservatives.

### Treating Cancer

Provided herein are methods of killing a plurality of cancer cells, or treating cancer, in a subject, comprising administering to the subject a therapeutically effective amount of a composition comprising immune cells comprising the first and second receptors of the disclosure. The immune cells express both receptors in the same cell.

In some embodiments, the plurality of cancer cells express the target antigen. In some embodiments, the plurality cancer cells of the subject express CEA. CEA positive cancers include colorectal cancer, pancreatic cancer, esophageal cancer, gastric cancer, lung adenocarcinoma, head and neck cancer, diffuse large B cell cancer or acute myeloid leukemia cancer. In some embodiments, the colorectal cancer is a high microsatellite instability colorectal cancer.

In some embodiments, a plurality of cancer cells as low or no expression of BEST2, BEST4, SCARA5, EPHA7, TGFBR2, or a peptide antigen of BEST2, BEST4, SCARA5, EPHA7, or TGFBR2.

The disclosure provides methods of treating a cancer in a subject comprising measuring the expression level of the non-target antigen in a plurality of cancer cells, and treating the subject when the expression level of the non-target antigen in the plurality of cancer cells is less than the expression level of the non-target antigen in the plurality of cancer cells is less than the expression level of the non-target antigen a plurality of healthy cells. In some embodiments, the non-target antigen comprises BEST2, BEST4, SCARA5, EPHA7, or TGFBR2, or a peptide antigen of BEST2, BEST4, SCARA5, EPHA7, or TGFBR2. In some embodiments, the methods comprise determining the expression of CEA in a plurality of cancer cells; and administering a plurality of immune cells to the subject if the plurality of cancer cells have low or no expression of the non-target antigen, and the plurality of cancer cells are CEA positive.

Methods of measuring the expression of the target antigen in cancer or wild type cells from a subject will be readily apparent to persons of ordinary skill in the art. These include, inter alia, methods of measuring RNA expression such as RNA sequencing and reverse transcription polymerase chain reaction (RT-PCR), as well as methods of measuring protein expression such as immunohistochemistry based methods.

In some embodiments, the first ligand comprises IMIGVLVGV (SEQ ID NO: 2). In some embodiments, the first ligand is complexed with a major histocompatibility complex comprising a human leukocyte antigen A*02 allele (HLA-A*02).

In some embodiments, the immune cells are T cells.

In some embodiments, the immune cells are allogeneic or autologous.

In some embodiments, the second receptor increases the specificity of the immune cells for the CEA positive cancer cells compared to immune cells that express the first receptor but do not express the second receptor. In some embodiments, the immune cells have reduced side effects compared to immune cells that express the first receptor but do not express the second receptor.

### Kits and Articles of Manufacture

The disclosure provides kits and articles of manufacture comprising the polynucleotides and vectors encoding the receptors described herein, and immune cells comprising the receptors described herein. In some embodiments, the kit comprises articles such as vials, syringes and instructions for use.

In some embodiments, the kit comprises a polynucleotide or vector comprising a sequence encoding one or more receptors of the disclosure.

In some embodiments, the kit comprises a plurality of immune cells comprising the first and second receptors as described herein. In some embodiments, the plurality of immune cells comprises a plurality of T cells.

In some embodiments, the kit further comprises instructions for use.

### EXAMPLES

### Example 1: Identification of Transmembrane Proteins as Candidate Blockers

This study identified protein candidates to use as non-target antigens. Non-target antigens are bound by a non-target antigen receptor in T cells, and act as a mechanism to block activation of the T cell that would otherwise induced by a target antigen receptor. The approach to identify non-target antigens included using a bioinformatics pipeline to identify proteins with low or no expression in cancer cell lines and a relatively greater level of expression in normal tissue, in particular colorectal tissues. In this example, the informatics pipeline was used to identify transmembrane proteins with low or no expression in colorectal cancer cells and a higher level of expression in healthy colon tissues. The ratio of the expression levels in healthy colon cells and cancerous colon cells was calculated for each transmembrane protein to identify candidate non-target antigens for use as a non-target antigen.

Bioinformatics Pipeline

An informatics workflow was developed to identify candidate transmembrane proteins expressed in colon tissue (FIG. 1). The first step was to mine Uniprot for potential transmembrane proteins in the human proteome. We analyzed 20,365 proteins expressed in human colon tissue in Uniprot and identified 5,177 transmembrane proteins that were expressed in the colon. This set of transmembrane proteins served as an initial list of targets to evaluate in the subsequent analysis. Next, RNAseq data from the GTEx database was analyzed for expression levels of the candidate transmembrane proteins in healthy colon tissue. The GTEx project is a public resource that contains tissue-specific gene expression data from 54 healthy tissue sites across nearly 1000 individuals. In order to be selected for further evaluation, expression of the gene encoding the transmembrane protein had to be at least 5 transcripts per kilobase million (TPM). Among the 339 normal colon tissues samples in the GTEx database, there were 2,439 genes that met this criteria. The RNAseq data was retrieved for these genes. Next, RNAseq data from The Cancer Genome Atlas (TCGA) database was analyzed. The TCGA is a resource for over 20,000 primary cancer and matched normal samples spanning 33 cancer types, and is available from the National Cancer Institute and the National Human Genome Research Institute. The TCGA colon adenocarcinoma and rectum adenocarcinoma (TCGA-COADREAD) dataset was mined for the 2,439 genes that met the GTEx criteria. RNAseq data was curated from 372 samples in TCGA-COADREAD for tumor tissue and adjacent normal tissue. The RNASeq datasets procured from the GTEx and TCGA databases were combined into a single in-house database. Expression data were normalized using the FPKM method (Trapnell et al. Nat Biotechnol. 28:511-515 (2010)) for further analysis.

In order to filter out non-target antigens that are expressed in colon cancer cells, the list of candidate genes was compared to the Cancer Cell Line Encyclopedia (CCLE). The CCLE project database houses detailed genetic and pharmacologic characterization of human cancer models. RNAseq expression data was procured from the CCLE database for 57 colorectal cancer (CRC) cell lines. Expression of the 2,439 genes identified in the GTEx database was evaluated in the 57 CRC cell lines. Inclusion criteria for a gene as a potential non-target antigen required that the gene be expressed in normal in healthy cells and have low or no expression in colorectal cancer cells. Therefore, the criteria for selecting candidate genes as a non-target antigen was low or no expression in over 50% of the 57 cancer cell lines, defined as having less than 1 fragment per kilobase million (FPKM) of the gene present in the RNAseq data housed in the CCLE. A total of 2,312 genes met this criteria. Of these, 2,188 genes had low or no expression in all of the CRC cell lines.

Comparison of Expression in Normal and Cancer Tissue

To establish an additional filter for candidate non-target antigens, a threshold measure of expression was determined by comparing the FPKM normalized levels of expression in normal tissue (GTEx) and cancerous tissue (TCGA). A ratio of the GTEx:TCGA expression levels was calculated for each gene. Inclusion criteria for a gene to be considered a potential non-target antigen was a GTEx:TCGA ratio of at least 5. Of the 2,312 genes analyzed, 180 had a GTEx:TCGA expression ratio of 5 or more.

Further Considerations for Candidate Non-target Antigens

The candidate gene list was further filtered by other factors, such as cellular localization and tissue localization. Transmembrane proteins that may not be accessible to the cellular therapy are those not expressed on the cell surface and those not exposed to the blood. For example, transmembrane proteins expressed in membranous intracellular compartments, such as the golgi apparatus, may not be expressed on the cell surface. Transmembrane proteins likely to be expressed on the apical (luminal) side of the colon will not be accessible to the blood. Genes with probable high expression in off-target tissues, such as nerve, muscle, or immune cells were also considered unlikely candidates because they could represent contamination in the RNAseq data.

Results

A list of exemplary candidate genes resulting from the informatics pipeline are listed in Table 4. The Table shows a subset of the non-target candidates that maybe used as the non-target antigen in the cell therapy described herein.

TABLE 4
Candidate Non-Target Antigens

| | | % CRC cell | |
|---|---|---|---|
| | | lines with | GTEx Normal:TCGA |
| Uniprot ID | Gene names | <1 RPKM | Tumor Ratio (FPKM) |
| Q8NFU1 | BEST2 | 98 | 108.4 |
| Q8NFU0 | BEST4 | 95 | 76.0 |
| Q6ZMJ2 | SCARA5 | 98 | 60.9 |
| Q15375 | EPHA7 | 95 | 23.1 |

### Example 2: Identification of TGFBR2 as a Candidate Blocker

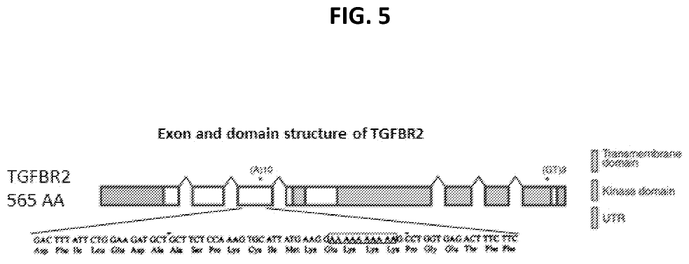

Approximately 15% of colorectal cancers (CRCs) present with a high microsatellite instability (MSI-H) phenotype. A 10-adenine microsatellite is present in the third exon of TGFBR2 (see FIG. 5). Approximately 80% of microsatellite instability high (MSI-H) CRCs contain biallelic mutations which typically result in frameshift mutations that produce a truncated TGFBR2 protein that lacks a transmembrane domain. CRCs with truncated TGFBR2 thus lack would lack expression of the truncated portion of the protein, a potential blocker target. In addition, truncated proteins may be targeted for degradation, reducing the level of even truncated TGFBR2 protein products. Frameshift mutations in the TGFRBR2 microsatellite region occur in both heredity non adenomatous polyposis coli (APC) and spontaneous CRCs. Pairing a TGFBR2 blocker with a CEA activator thus has the potential treat MSI-H CRC.

What is claimed is:

1. An immune cell responsive to low or no expression of a protein in a cancer cell, comprising:
a. a first receptor, optionally a chimeric antigen receptor (CAR) or T cell receptor (TCR), comprising an extracellular ligand binding domain specific to a target antigen selected from:
i. a cancer cell-specific antigen, or a peptide antigen thereof in a complex with a major histocompatibility complex class I (MHC-I); or
ii. CEA cell adhesion molecule 5 (CEA), or a peptide antigen thereof in a complex with a major histocompatibility complex class I (MHC-I); and
b. a second receptor, optionally an inhibitory chimeric antigen receptor (iCAR), comprising an extracellular ligand binding domain specific to a non-target antigen selected from bestrophin-2 (BEST2), bestrophin-4 (BEST4), scavenger receptor class A member 5 (SCARA5), ephrin type-A receptor 7 (EPHA7) and transforming growth factor beta receptor 2 (TGFBR2), or an antigen peptide thereof in a complex with a major histocompatibility complex class I (MHC-I), wherein the non-target antigen is expressed at a lower level by a population of target cells than by a population of non-target cells.

2. The immune cell of claim 1, wherein the target antigen is a cancer cell-specific antigen.

3. The immune cell of claim 1 or 2, wherein the target antigen is a peptide antigen of a cancer cell-specific antigen in a complex with a major histocompatibility complex class I (MHC-I).

4. The immune cell of any one of claims 1-3, wherein the cancer cell is a colorectal cancer cell.

5. The immune cell of any one of claims 1-3, wherein the cancer cell is a pancreatic cancer cell, esophageal cancer cell, gastric cancer cell, lung adenocarcinoma cell, head-and-neck cancer cell, diffuse large B cell cancer cell, or acute myeloid leukemia cancer cell.

6. The immune cell of any one of claims 1-5, wherein the target antigen is CEA.

7. The immune cell of any one of claims 1-5, wherein the target antigen is a peptide antigen of CEA in a complex with a major histocompatibility complex class I (MHC-I).

8. The immune cell of any one of claims 1-7, wherein the target antigen is expressed by a target cell.

9. The immune cell of any one of claims 1-8, wherein the non-target antigen is not expressed by the target cell.

10. The immune cell of any one of claims 1-9, wherein the non-target antigen is expressed by a non-target cell.

11. The immune cell of any one of claims 1-10, wherein the non-target cell expresses both the target antigen and the non-target antigen.

12. The method of claim 11, wherein the non-target cell is a healthy cell.

13. The immune cell of any one of claims 1-12, wherein the non-target antigen is expressed at a lower level by the target cell than the non-target cell.

14. The immune cell of any one of claims 1-12, wherein the non-target antigen expression level is at least about 10 times less, about 30 times less, about 50 times less, about 70 times less, about 90 times less, about 100 times less, or about 110 times less in the target cell than in the non-target cell.

15. The immune cell of any one of claims 1-12, wherein the non-target antigen expression level is at least about 5 times less in the target cell than in the non-target cell.

16. The method of any one of claims 13-15, wherein the non-target cell is a colon cell.

17. The immune cell of any one of claims 1-16, wherein the first receptor and the second receptor together specifically activate the immune cell in the presence of the target cell.

18. The immune cell of any one of claims 1-17, wherein the immune cell is a T cell.

19. The immune cell of claim 18, wherein the immune cell is a CD8+ CD4− T cell.

20. The immune cells of any one of claims 1-19, wherein the target cells are CEA positive cancer cells.

21. The immune cell of claim 20, wherein the CEA positive cancer cells comprise colorectal cancer cells, pancreatic cancer cells, esophageal cancer cells, gastric cancer cells, lung adenocarcinoma cells, head and neck cancer cells, diffuse large B cell cancer cells or acute myeloid leukemia cancer cells.

22. The immune cell of any one of claims 1-21, wherein the CEA comprises a sequence that shares at least 95% identity to SEQ ID NO: 1.

23. The immune cell of any one of claims 1-21, wherein the peptide antigen of CEA is IMIGVLVGV (SEQ ID NO: 2).

24. The immune cell of any one of claims 1-23, wherein the MHC-I comprises a human leukocyte antigen A*02 allele (HLA-A*02).

25. The immune cell of any one of claims 1-24, wherein the first receptor is a T cell receptor (TCR).

26. The immune cell of any one of claims 1-24, wherein the first receptor is a chimeric antigen receptor (CAR).

27. The immune cell of claim 25 or 26, wherein the extracellular ligand binding domain of the first receptor comprises an antibody fragment, a single chain Fv antibody fragment (ScFv), or a β chain variable domain (Vβ).

28. The immune cell of claim 25 or 26, wherein the extracellular ligand binding domain of the first receptor comprises a TCR α chain variable domain and a TCR β chain variable domain.

29. The immune cell of claim 28, wherein the extracellular ligand binding domain of the first receptor comprises complement determining regions (CDRs) selected from SEQ ID NOs: 3-12.

30. The immune cell of claim 29, wherein:
a. the TCR α chain variable domain comprises a CDR-1 of TSITA (SEQ ID NO: 3), a CDR-2 of IRSNER (SEQ ID NO: 4) and a CDR-3 comprising ATDLTSGGNYK (SEQ ID NO: 5), ATDFTSGGNYK (SEQ ID NO: 6), ATDLTTGGNYK (SEQ ID NO: 7) or ATDFTTGGNYK (SEQ ID NO: 8); and
b. the TCR β chain variable domain comprises a CDR-1 of KGHPV (SEQ ID NO: 9), a CDR-2 of FQNQEV (SEQ ID NO: 10), and a CDR-3 of ASSLGLGDYEQ (SEQ ID NO: 11) or ASSLGTGDYEQ (SEQ ID NO: 12).

31. The immune cell of claim 29, wherein:
a. the TCR α chain variable domain comprises a CDR-1 of SEQ ID NO: 9, a CDR-2 of SEQ ID NO: 10 and a CDR-3 of SEQ ID NO: 11 or SEQ ID NO: 12; and
b. the TCR β chain variable domain comprises a CDR-1 of SEQ ID NO: 3, a CDR-2 of SEQ ID NO: 4 and a CDR-3 comprising SEQ ID NO: 5, SEQ ID NO: 6, SEQ ID NO: 7 or SEQ ID NO: 8.

32. The immune cell of claim 27, wherein the ScFv comprises CDRs selected from SEQ ID NOs: 55-63.

33. The immune cell of claim 32, wherein the ScFv comprises a CDR-H1 of EFGMN (SEQ ID NO: 55), a CDR-H2 of WINTKTGEATYVEEFKG (SEQ ID NO: 56), a CDR-H3 of WDFAYYVEAMDY (SEQ ID NO: 57) or WDFAHYFQTMDY (SEQ ID NO: 58), a CDR-L1 of KASQNVGTNVA (SEQ ID NO: 59) or KASAAVGTYVA (SEQ ID NO: 60), a CDR-L2 of SASYRYS (SEQ ID NO: 61) or SASYRKR, and a CDR-L3 of HQYYTYPLFT (SEQ ID NO: 63).

34. The immune cell of claim 27, wherein the ScFv comprises a sequence selected from SEQ ID NOs: 64-70 or a sequence having at least 85%, at least 90%, at least 95%, at least 97% or at least 99% identity thereto.

35. The immune cell of claim 27, wherein the ScFv comprises a sequence selected from SEQ ID NOs: 64-70.

36. A pharmaceutical composition, comprising a therapeutically effective amount of the immune cells of any one of claims 1-35.

37. The pharmaceutical composition of claim 36, further comprising a pharmaceutically acceptable carrier, diluent or excipient.

38. The pharmaceutical composition of claim 36 or 37, for use as a medicament in the treatment of cancer.

39. A polynucleotide system, comprising one or more polynucleotides comprising polynucleotide sequences encoding:
a. a first receptor, optionally a chimeric antigen receptor (CAR) or T cell receptor (TCR), comprising an extracellular ligand binding domain specific to a target antigen selected from:
i. a cancer cell-specific antigen, or a peptide antigen thereof in a complex with a major histocompatibility complex class I (MHC-I); or
ii. CEA cell adhesion molecule 5 (CEA), or a peptide antigen thereof in a complex with a major histocompatibility complex class I (MHC-I); and
b. a second receptor, optionally an inhibitory chimeric antigen receptor (iCAR), comprising an extracellular ligand binding domain specific to a non-target antigen selected from BEST2, BEST4, SCARA5, EPHA7, TGFBR2, or an antigen peptide thereof in a complex with a major histocompatibility complex class I (MHC-I), wherein the non-target antigen is expressed at a lower level by a population of target cells than by a population of non-target cells.

40. A vector, comprising the one or more polynucleotides of claim 39.

41. A method of killing a plurality of cancer cells and/or treating cancer in a subject, comprising administering to the subject an effective amount of the immune cell of any one of claims 1 to 35 or the pharmaceutical composition of any one of claims 36-38.

42. The method of claim 41, wherein a plurality of cancer cells express the target antigen.

43. The method of claim 41 or 42, wherein the plurality of cancer cells do not express the non-target antigen.

44. The method of claim 41 or 42, wherein the plurality of cancer cells express the non-target antigen at a lower level than a plurality of healthy cells.

45. The method of claim 44, wherein the plurality of healthy cells express both the target antigen and the non-target antigen.

46. The method of claim 44 or 45, wherein the plurality of cancer cells express the non-target antigen at a level that is at least about 10 times less, about 30 times less, about 50 times less, about 70 times less, about 90 times less, about 100 times less, or about 110 times less than the plurality of healthy cells.

47. The method of claim 44 or 45, wherein the non-target antigen expression level is at least about 5 times less in the plurality of cancer cells than in the plurality of healthy cells.

48. The method of any one of claims 44-47, wherein the plurality of healthy cells comprises colon cells.

49. The method of any one of claims 41-48, further comprising measuring the expression level of the non-target antigen in a plurality of cancer cells, and treating the subject when the expression level of the non-target antigen in the plurality of cancer cells is less than the expression level of the non-target antigen in the plurality of cancer cells is less than the expression level of the non-target antigen a plurality of healthy cells.

50. A method of making a plurality of immune cells, comprising:
a. providing a plurality of immune cells, and
b. transforming the plurality of immune cells with the polynucleotide system of claim 39 or the vector of claim 40.

51. A kit comprising the immune cell of any one of claims 1 to 35 or the pharmaceutical composition of any one of claims 36-38.

52. The kit of claim 51, further comprising instructions for use.

53. A TCR comprising:
(1) a TCR alpha chain comprising or consisting essentially of amino acids 1-270 of any one of SEQ ID NOS: 16-31, or a sequence at least 95% identical thereto; and
(2) a TCR beta chain comprising or consisting essentially of amino acids 293-598 of any one of SEQ ID NOS: 16-31, or a sequence at least 95% identical thereto.

54. A TCR comprising:
a. a TCR alpha chain comprising amino acids 1-270 of SEQ ID NO: 16 and a TCR beta chain comprising amino acids 293-598 of SEQ ID NO: 16;
b. a TCR alpha chain comprising amino acids 1-270 of SEQ ID NO: 17 and a TCR beta chain comprising amino acids 293-598 of SEQ ID NO: 17;
c. a TCR alpha chain comprising amino acids 1-270 of SEQ ID NO: 18 and a TCR beta chain comprising amino acids 293-598 of SEQ ID NO: 18;
d. a TCR alpha chain comprising amino acids 1-270 of SEQ ID NO: 19 and a TCR beta chain comprising amino acids 293-598 of SEQ ID NO: 19;
e. a TCR alpha chain comprising amino acids 1-270 of SEQ ID NO: 20 and a TCR beta chain comprising amino acids 293-598 of SEQ ID NO: 20;
f. a TCR alpha chain comprising amino acids 1-270 of SEQ ID NO: 21 and a TCR beta chain comprising amino acids 293-598 of SEQ ID NO: 21;
g. a TCR alpha chain comprising amino acids 1-270 of SEQ ID NO: 22 and a TCR beta chain comprising amino acids 293-598 of SEQ ID NO: 22;
h. a TCR alpha chain comprising amino acids 1-270 of SEQ ID NO: 23 and a TCR beta chain comprising amino acids 293-598 of SEQ ID NO: 23;
i. a TCR alpha chain comprising amino acids 1-270 of SEQ ID NO: 24 and a TCR beta chain comprising amino acids 293-598 of SEQ ID NO: 24;
j. a TCR alpha chain comprising amino acids 1-270 of SEQ ID NO: 25 and a TCR beta chain comprising amino acids 293-598 of SEQ ID NO: 25;
k. a TCR alpha chain comprising amino acids 1-270 of SEQ ID NO: 26 and a TCR beta chain comprising amino acids 293-598 of SEQ ID NO: 26;
l. a TCR alpha chain comprising amino acids 1-270 of SEQ ID NO: 27 and a TCR beta chain comprising amino acids 293-598 of SEQ ID NO: 27;
m. a TCR alpha chain comprising amino acids 1-270 of SEQ ID NO: 28 and a TCR beta chain comprising amino acids 293-598 of SEQ ID NO: 28;
n. a TCR alpha chain comprising amino acids 1-270 of SEQ ID NO: 29 and a TCR beta chain comprising amino acids 293-598 of SEQ ID NO: 29;
o. a TCR alpha chain comprising amino acids 1-270 of SEQ ID NO: 30 and a TCR beta chain comprising amino acids 293-598 of SEQ ID NO: 30; or
p. a TCR alpha chain comprising amino acids 1-270 of SEQ ID NO: 31 and a TCR beta chain comprising amino acids 293-598 of SEQ ID NO: 31.

55. An immune cell, comprising the TCR of claim 53 or 54.

56. The immune cell of claim 55, further comprising a second receptor, optionally an inhibitory chimeric antigen receptor (iCAR), comprising an extracellular ligand binding domain specific to a non-target antigen selected from BEST2, BEST4, SCARA5, EPHA7, and TFGBR2, or an antigen peptide of any of these in a complex with a major histocompatibility complex class I (MHC-I).
